(19) United States
(12) Patent Application Publication
(10) Pub. No.: US 2020/0338210 A1
(43) Pub. Date: Oct. 29, 2020

(54) ANTI-ROR1 ANTIBODY AND CONJUGATES THEREOF

(71) Applicant: Ardeagen Corporation, Woodinville, WA (US)

(72) Inventor: Morris ROSENBERG, Woodinville, WA (US)

(21) Appl. No.: 16/471,813

(22) PCT Filed: Dec. 21, 2017
(86) PCT No.: PCT/US17/68026

§ 371 Date: Jun. 20, 2019

Related U.S. Application Data
(60) Provisional application No. 62/438,042, filed on Dec. 22, 2016.

(51) Int. Cl. A61K 47/68; C07K 16/28; A61P 35/00

(52) U.S. Cl. CPC A61K 47/6849; A61K 47/6803; C07K 16/2803; C07K 2317/565; C07K 2317/24; C07K 2317/77; C07K 2317/73; A61P 35/00

(57) ABSTRACT

Antibody-payload conjugates are used for directed delivery of a cytotoxic payload to a cancer via use of target-specific antibody. Disclosed herein are compositions and methods of treating a cancer, which comprises a pharmaceutical composition comprising an anti-ROR1 antibody-payload conjugate.

### CROSS-REFERENCE

This application claims the benefit of U.S. Provisional Application No. 62/438,042, filed Dec. 22, 2016, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 12, 2017, is named 45926-704_601_SL.txt and is 50,026 bytes in size.

### BACKGROUND OF THE DISCLOSURE

Cancer is a heterogenous disease. in some instances, one or more specific indications share a cancer marker. Antibody-drug conjugates (ADCs) combine the binding specificity of monoclonal antibodies with the potency of chemotherapeutic agents. in some instances, an antibody-drug conjugate is utilized for specific targeting of a cancer marker for treatment.

### SUMMARY OF THE DISCLOSURE

Disclosed herein, in certain embodiments, are anti-ROR1 antibody-payload conjugates, and pharmaceutical compositions. In some embodiments, also included herein are methods of treatment utilizing an anti-ROR1 antibody-payload conjugate described herein.

Disclosed herein, in certain embodiments, is a method of treating a subject having cancer, comprising: administering to the subject a therapeutically effective amount of a pharmaceutical composition comprising an anti-ROR1 antibody-payload conjugate wherein the subject has bladder cancer, breast cancer, colorectal cancer, liver cancer, lung cancer, pancreatic cancer, renal cell carcinoma, stomach cancer, adrenal cancer, skin cancer, prostate cancer, B-cell lymphoma or acute lymphoblastic leukemia. In some embodiments, the payload comprises an auristatin derivative, maytansine, a maytansinoid, a taxane, a calicheamicin, cemadotin, a duocarmycin, a pyrrolobenzodiazepine (PBD), a tubulysin, or a combination thereof. In some embodiments, the auristatin derivative is monomethyl auristatin E (MMAE). In some embodiments, the auristatin derivative is monomethyl auristatin F (MMAF). In some embodiments, the maytansinoid comprises DM1 (mertansine) or DM4. In some embodiments, the pyrrolobenzodiazepine is a pyrrolobenzodiazepine dimer. In some embodiments, the payload comprises MMAE. In some embodiments, the payload comprises maytansine. In some embodiments, the payload comprises a pyrrolobenzodiazepine dimer. In some embodiments, the anti-ROR1 antibody-payload conjugate further comprises a linker moiety that attaches the anti-ROR1 antibody to the payload. In some embodiments, the linker moiety comprises a homobifunctional linker or a heterobifunctional linker. In some embodiments, the linker moiety comprises a cleavable linker. In some embodiments, the linker moiety comprises a non-cleavable linker. In some embodiments, the linker moiety comprises a valine-citrulline moiety. In some embodiments, the linker moiety further comprises p-aminobenzoic acid. In some embodiments, the anti-ROR1 antibody further comprises a formylglycine residue generated by a formylglycine-generating enzyme. In some embodiments, the payload is conjugated to the anti-ROR1 antibody at the formylglycine site. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 3, (ii) a variable heavy (VH) CDR2 that has an amino acid sequence of SEQ ID NO: 4, and (iii) a variable heavy (VH) CDR3 that has an amino acid sequence of SEQ ID NO: 5, and wherein the light chain variable region comprises (iv) a variable light (VL) CDR 1 that has an amino acid sequence of SEQ ID NO: 6, (v) a variable light (VL) CDR 2 that has an amino acid sequence of SEQ ID NO: 7, and (vi) a variable light (VL) CDR 3 that has an amino acid sequence of SEQ ID NO: 8. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 20, (ii) a variable heavy (VH) CDR2 that has an amino acid sequence of SEQ ID NO: 21, and (iii) a variable heavy (VH) CDR3 that has an amino acid sequence of SEQ ID NO: 22, and wherein the light chain variable region comprises (iv) a variable light (VL) CDR 1 that has an amino acid sequence of SEQ ID NO: 23, (v) a variable light (VL) CDR 2 that has an amino acid sequence of SEQ ID NO: 24, and (vi) a variable light (VL) CDR 3 that has an amino acid sequence of SEQ ID NO: 25. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 30, (ii) a variable heavy (VH) CDR2 that has an amino acid sequence of SEQ ID NO: 31, and (iii) a variable heavy (VH) CDR3 that has an amino acid sequence of SEQ ID NO: 32, and wherein the light chain variable region comprises (iv) a variable light (VL) CDR 1 that has an amino acid sequence of SEQ ID NO: 33, (v) a variable light (VL) CDR 2 that has an amino acid sequence of SEQ ID NO: 34, and (vi) a variable light (VL) CDR 3 that has an amino acid sequence of SEQ ID NO: 35. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 38, (ii) a variable heavy (VH) CDR2 that has an amino acid sequence of SEQ ID NO: 39, and; (iii) a variable heavy (VH) CDR3 that has an amino acid sequence of SEQ ID NO: 40, and wherein the light chain variable region comprises (iv) a variable light (VL) CDR 1 that has an amino acid sequence of SEQ ID NO: 41, (v) a variable light (VL) CDR 2 that has an amino acid sequence of SEQ ID NO: 42, and (vi) a variable light (VL) CDR 3 that has an amino acid sequence of SEQ ID NO: 43. In some embodiments, the anti-ROR1 antibody comprises a heavy chain variable region having at least 80%, 85%, 90%, 95%, or 99% sequence identity to SEQ ID NO: 1, 9, or 13 and a light chain variable region having at least 80%, 85%, 90%, 95% or 99% sequence identity to SEQ ID NO: 2, 10 or 14. In some embodiments, the anti-ROR1 antibody comprises a heavy chain variable region of SEQ ID NO: 1 and a light chain variable region of SEQ ID NO: 2. In some embodiments, the anti-ROR1 antibody comprises a heavy chain variable region of SEQ ID NO: 9 and a light chain variable region of SEQ ID NO: 10. In some embodiments, the anti-ROR1 antibody comprises a heavy chain variable region of SEQ ID NO: 13 and a light chain variable region of SEQ ID NO: 14. In some embodiments, the anti-ROR1 antibody comprises a heavy chain variable region having at least 80%, 85%, 90%, 95%, or 99% sequence identity to SEQ ID NO: 17 or 18 and a light chain variable region having at least 80%, 85%, 90%, 95%, or 99% sequence identity to SEQ ID NO: 19. In some embodiments, the anti-ROR1 antibody comprises a heavy chain variable region of SEQ ID NO: 17 and a light chain variable region of SEQ ID NO: 19. In some embodiments, the anti-ROR1 antibody comprises a heavy chain variable region of SEQ ID NO: 18 and a light chain variable region of SEQ ID NO: 19. In some embodiments, the anti-ROR1 antibody comprises a heavy chain variable region having at least 80%, 85%, 90%, 95%, or 99% sequence identity to SEQ ID NO: 28 and a light chain variable region having at least 80%, 85%, 90%, 95%, or 99% sequence identity to SEQ ID NO: 29. In some embodiments, the anti-ROR1 antibody comprises a heavy chain variable region of SEQ ID NO: 28 and a light chain variable region of SEQ ID NO: 29. In some embodiments, the anti-ROR1 antibody comprises a heavy chain variable region having at least 80%, 85%, 90%, 95%, or 99% sequence identity to SEQ ID NO: 36 and a light chain variable region having at least 80%, 85%, 90%, 95%, or 99% sequence identity to SEQ ID NO: 37. In some embodiments, the anti-ROR1 antibody comprises a heavy chain variable region of SEQ ID NO: 36 and a light chain variable region of SEQ ID NO: 37. In some embodiments, the anti-ROR1 antibody-payload conjugate comprises about 2, 3, 4, 5, 6, or more payloads. In some embodiments, the anti-ROR1 antibody-payload conjugate comprises about 4 payloads. In some embodiments, the anti-ROR1 antibody-payload conjugate comprises about 2 payloads. In some embodiments, the payloads are the same. In some embodiments, the payloads are different. In some embodiments, the B-cell lymphoma comprises Hodgkin's lymphoma. In some embodiments, the B-cell lymphoma comprises non-Hodgkin's lymphoma. In some embodiments, the non-Hodgkin's lymphoma comprises diffuse large B-cell lymphoma (DLBCL), follicular lymphoma, marginal zone B-cell lymphoma (MZL), chronic lymphocytic leukemia (CLL), small lymphocytic lymphoma (SLL), Mantle cell lymphoma (MCL), Burkitt's lymphoma, Waldenstrom's macroglobulinemia, nodal marginal zone B cell lymphoma (NMZL), splenic marginal zone lymphoma (SMZL), intravascular large B-cell lymphoma, primary effusion lymphoma, lymphomatoid granulomatosis, primary central nervous system lymphoma or plasmablastic lymphoma. In some embodiments, the pharmaceutical composition further comprises an excipient. In some embodiments, the method further comprises an additional therapeutic agent. In some embodiments, the additional therapeutic agent comprises an antimetabolite, an intercalating agent, a platinum derivative, an alkylating agent, an antimitotic agent, a topoisomerase inhibitor, a cell cycle inhibitor, an immune system checkpoint inhibitor, or a microtubule agent. In some embodiments, the subject has breast cancer. In some embodiments, the subject has lung cancer. In some embodiments, the subject has liver cancer. In some embodiments, the subject has stomach cancer. In some embodiments, the subject is a human.

Disclosed herein, in certain embodiments, is a method of treating a subject having liver cancer, comprising: administering to the subject having liver cancer a therapeutically effective amount of a pharmaceutical composition comprising an anti-ROR1 antibody-payload conjugate wherein the anti-ROR1 antibody is selected from 2A2, R11, R12, and Y31.

Disclosed herein, in certain embodiments, is a method of treating a subject having liver cancer, comprising: administering to the subject having liver cancer a therapeutically effective amount of a pharmaceutical composition comprising an anti-ROR1 antibody-payload conjugate wherein the anti-ROR1 antibody recognizes an epitope located within the immunoglobulin (Ig) domain, the Frizzled domain, or the Kringle domain of human ROR1. In some embodiments, the payload comprises an auristatin derivative, maytansine, a maytansinoid, a taxane, a calicheamicin, cemadotin, a duocarmycin, a pyrrolobenzodiazepine (PBD), a tubulysin, or a combination thereof. In some embodiments, the auristatin derivative is monomethyl auristatin E (MMAE). In some embodiments, the auristatin derivative is monomethyl auristatin F (MMAF). In some embodiments, the maytansinoid comprises DM1 (mertansine) or DM4. In some embodiments, the pyrrolobenzodiazepine is a pyrrolobenzodiazepine dimer. In some embodiments, the payload comprises MMAE. In some embodiments, the payload comprises maytansine. In some embodiments, the payload comprises a pyrrolobenzodiazepine dimer. In some embodiments, the anti-ROR1 antibody-payload conjugate further comprises a linker moiety that attaches the anti-ROR1 antibody to the payload. In some embodiments, the linker moiety comprises a homobifunctional linker or a heterobifunctional linker. In some embodiments, the linker moiety comprises a cleavable linker. In some embodiments, the linker moiety comprises a non-cleavable linker. In some embodiments, the linker moiety comprises a valine-citrulline moiety. In some embodiments, the linker moiety further comprises p-aminobenzoic acid. In some embodiments, the anti-ROR1 antibody further comprises a formylglycine residue generated by a formylglycine-generating enzyme. In some embodiments, the payload is conjugated to the anti-ROR1 antibody at the formylglycine site. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 3, (ii) a variable heavy (VH) CDR2 that has an amino acid sequence of SEQ ID NO: 4, and (iii) a variable heavy (VH) CDR3 that has an amino acid sequence of SEQ ID NO: 5, and wherein the light chain variable region comprises (iv) a variable light (VL) CDR 1 that has an amino acid sequence of SEQ ID NO: 6, (v) a variable light (VL) CDR 2 that has an amino acid sequence of SEQ ID NO: 7, and (vi) a variable light (VL) CDR 3 that has an amino acid sequence of SEQ ID NO: 8. In some embodiments, anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 20, (ii) a variable heavy (VH) CDR2 that has an amino acid sequence of SEQ ID NO: 21, and (iii) a variable heavy (VH) CDR3 that has an amino acid sequence of SEQ ID NO: 22, and wherein the light chain variable region comprises (iv) a variable light (VL) CDR 1 that has an amino acid sequence of SEQ ID NO: 23, (v) a variable light (VL) CDR 2 that has an amino acid sequence of SEQ ID NO: 24, and (vi) a variable light (VL) CDR 3 that has an amino acid sequence of SEQ ID NO: 25. In some embodiments, anti-ROR1 antibody comprises a heavy chain variable region having at least 80%, 85%, 90%, 95%, or 99% sequence identity to SEQ ID NO: 1, 9, or 13 and a light chain variable region having at least 80%, 85%, 90%, 95% or 99% sequence identity to SEQ ID NO: 2, 10 or 14. In some embodiments, anti-ROR1 antibody comprises a heavy chain variable region of SEQ ID NO: 1 and a light chain variable region of SEQ ID NO: 2. In some embodiments, anti-ROR1 antibody comprises a heavy chain variable region of SEQ ID NO: 9 and a light chain variable region of SEQ ID NO: 10. In some embodiments, anti-ROR1 antibody comprises a heavy chain variable region of SEQ ID NO: 13 and a light chain variable region of SEQ ID NO: 14. In some embodiments, anti-ROR1 antibody comprises a heavy chain variable region having at least 80%, 85%, 90%, 95%, or 99% sequence identity to SEQ ID NO: 17 or 18 and a light chain variable region having at least 80%, 85%, 90%, 95%, or 99% sequence identity to SEQ ID NO: 19. In some embodiments, anti-ROR1 antibody comprises a heavy chain variable region of SEQ ID NO: 17 and a light chain variable region of SEQ ID NO: 19. In some embodiments, anti-ROR1 antibody comprises a heavy chain variable region of SEQ ID NO: 18 and a light chain variable region of SEQ ID NO: 19. In some embodiments, the anti-ROR1 antibody-payload conjugate comprises about 2, 3, 4, 5, 6, or more payloads. In some embodiments, the anti-ROR1 antibody-payload conjugate comprises about 4 payloads. In some embodiments, the payloads are the same. In some embodiments, the payloads are different. In some embodiments, the liver cancer is a metastatic liver cancer. In some embodiments, the liver cancer is a relapsed or a refractory liver cancer. In some embodiments, the pharmaceutical composition further comprises an excipient. In some embodiments, the method further comprises an additional therapeutic agent. In some embodiments, the additional therapeutic agent comprises an antimetabolite, an intercalating agent, a platinum derivative, an alkylating agent, an antimitotic agent, a topoisomerase inhibitor, a cell cycle inhibitor, or a microtubule inhibitor. In some embodiments, the subject is a human.

Disclosed herein, in certain embodiments, is an anti-ROR1 antibody comprising a heavy chain variable region having at least 80%, 85%, 90%, 95%, or 99% sequence identity to SEQ ID NO: 18 and a light chain variable region having at least 80%, 85%, 90%, 95%, or 99% sequence identity to SEQ ID NO: 19.

Disclosed herein, in certain embodiments, is a pharmaceutical composition comprising an anti-ROR1 antibody comprising a heavy chain variable region having at least 80%, 85%, 90%, 95%, or 99% sequence identity to SEQ ID NO: 18 and a light chain variable region having at least 80%, 85%, 90%, 95%, or 99% sequence identity to SEQ ID NO: 19, and an excipient.

Disclosed herein, in certain embodiments, is a nucleic acid polymer encoding an anti-ROR1 antibody comprising a heavy chain variable region having at least 80%, 85%, 90%, 95%, or 99% sequence identity to SEQ ID NO: 18 and a light chain variable region having at least 80%, 85%, 90%, 95%, or 99% sequence identity to SEQ ID NO: 19.

Disclosed herein, in certain embodiments, is an anti-ROR1 antibody-payload conjugate comprising an anti-ROR1 antibody conjugated to a payload, wherein the anti-ROR1 antibody recognizes an epitope located within the immunoglobulin (Ig) domain, the Frizzled domain, or the Kringle domain of human ROR1. In some embodiments, the payload comprises an auristatin derivative, maytansine, a maytansinoid, a taxane, a calicheamicin, cemadotin, a duocarmycin, a pyrrolobenzodiazepine (PBD), a tubulysin, or a combination thereof. In some embodiments, the auristatin derivative is monomethyl auristatin E (MMAE). In some embodiments, the auristatin derivative is monomethyl auristatin F (MMAF). In some embodiments, the maytansinoid comprises DM1 (mertansine) or DM4. In some embodiments, the pyrrolobenzodiazepine is a pyrrolobenzodiazepine dimer. In some embodiments, the payload comprises MMAE. In some embodiments, the payload comprises maytansine. In some embodiments, the payload comprises a pyrrolobenzodiazepine dimer. In some embodiments, the anti-ROR1 antibody-payload conjugate further comprises a linker moiety that attaches the anti-ROR1 antibody to the payload. In some embodiments, the linker moiety comprises a homobifunctional linker or a heterobifunctional linker. In some embodiments, the linker moiety comprises a cleavable linker. In some embodiments, the linker moiety comprises a non-cleavable linker. In some embodiments, the linker moiety comprises a valine-citrulline moiety. In some embodiments, the linker moiety further comprises p-aminobenzoic acid. In some embodiments, the anti-ROR1 antibody further comprises a formylglycine residue generated by a formylglycine-generating enzyme. In some embodiments, the payload is conjugated to the anti-ROR1 antibody at the formylglycine site. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 3, (ii) a variable heavy (VH) CDR2 that has an amino acid sequence of SEQ ID NO: 4, and (iii) a variable heavy (VH) CDR3 that has an amino acid sequence of SEQ ID NO: 5, and wherein the light chain variable region comprises (iv) a variable light (VL) CDR 1 that has an amino acid sequence of SEQ ID NO: 6, (v) a variable light (VL) CDR 2 that has an amino acid sequence of SEQ ID NO: 7, and (vi) a variable light (VL) CDR 3 that has an amino acid sequence of SEQ ID NO: 8. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 20, (ii) a variable heavy (VH) CDR2 that has an amino acid sequence of SEQ ID NO: 21, and (iii) a variable heavy (VH) CDR3 that has an amino acid sequence of SEQ ID NO: 22, and wherein the light chain variable region comprises (iv) a variable light (VL) CDR 1 that has an amino acid sequence of SEQ ID NO: 23, (v) a variable light (VL) CDR 2 that has an amino acid sequence of SEQ ID NO: 24, and (vi) a variable light (VL) CDR 3 that has an amino acid sequence of SEQ ID NO: 25. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 30, (ii) a variable heavy (VH) CDR2 that has an amino acid sequence of SEQ ID NO: 31, and (iii) a variable heavy (VH) CDR3 that has an amino acid sequence of SEQ ID NO: 32, and wherein the light chain variable region comprises (iv) a variable light (VL) CDR 1 that has an amino acid sequence of SEQ ID NO: 33, (v) a variable light (VL) CDR 2 that has an amino acid sequence of SEQ ID NO: 34, and (vi) a variable light (VL) CDR 3 that has an amino acid sequence of SEQ ID NO: 35. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 38, (ii) a variable heavy (VH) CDR2 that has an amino acid sequence of SEQ ID NO: 39, and; (iii) a variable heavy (VH) CDR3 that has an amino acid sequence of SEQ ID NO: 40, and wherein the light chain variable region comprises (iv) a variable light (VL) CDR 1 that has an amino acid sequence of SEQ ID NO: 41, (v) a variable light (VL) CDR 2 that has an amino acid sequence of SEQ ID NO: 42, and (vi) a variable light (VL) CDR 3 that has an amino acid sequence of SEQ ID NO: 43. In some embodiments, the anti-ROR1 antibody comprises a heavy chain variable region having at least 80%, 85%, 90%, 95%, or 99% sequence identity to SEQ ID NO: 1, 9, or 13 and a light chain variable region having at least 80%, 85%, 90%, 95% or 99% sequence identity to SEQ ID NO: 2, 10 or 14. In some embodiments, the anti-ROR1 antibody comprises a heavy chain variable region of SEQ ID NO: 1 and a light chain variable region of SEQ ID NO: 2. In some embodiments, the anti-ROR1 antibody comprises a heavy chain variable region of SEQ ID NO: 9 and a light chain variable region of SEQ ID NO: 10. In some embodiments, the anti-ROR1 antibody comprises a heavy chain variable region of SEQ ID NO: 13 and a light chain variable region of SEQ ID NO: 14. In some embodiments, the anti-ROR1 antibody comprises a heavy chain variable region having at least 80%, 85%, 90%, 95%, or 99% sequence identity to SEQ ID NO: 17 or 18 and a light chain variable region having at least 80%, 85%, 90%, 95%, or 99% sequence identity to SEQ ID NO: 19. In some embodiments, the anti-ROR1 antibody comprises a heavy chain variable region of SEQ ID NO: 17 and a light chain variable region of SEQ ID NO: 19. In some embodiments, the anti-ROR1 antibody comprises a heavy chain variable region of SEQ ID NO: 18 and a light chain variable region of SEQ ID NO: 19. In some embodiments, the anti-ROR1 antibody comprises a heavy chain variable region having at least 80%, 85%, 90%, 95%, or 99% sequence identity to SEQ ID NO: 28 and a light chain variable region having at least 80%, 85%, 90%, 95%, or 99% sequence identity to SEQ ID NO: 29. In some embodiments, the anti-ROR1 antibody comprises a heavy chain variable region of SEQ ID NO: 28 and a light chain variable region of SEQ ID NO: 29. In some embodiments, the anti-ROR1 antibody comprises a heavy chain variable region having at least 80%, 85%, 90%, 95%, or 99% sequence identity to SEQ ID NO: 36 and a light chain variable region having at least 80%, 85%, 90%, 95%, or 99% sequence identity to SEQ ID NO: 37. In some embodiments, the anti-ROR1 antibody comprises a heavy chain variable region of SEQ ID NO: 36 and a light chain variable region of SEQ ID NO: 37. In some embodiments, the anti-ROR1 antibody-payload conjugate comprises about 2, 3, 4, 5, 6, or more payloads. In some embodiments, the anti-ROR1 antibody-payload conjugate comprises about 4 payloads. In some embodiments, the payloads are the same. In some embodiments, the payloads are different.

Disclosed herein, in certain embodiments, is a pharmaceutical composition comprising an anti-ROR1 antibody-payload conjugate comprising an anti-ROR1 antibody conjugated to a payload, wherein the anti-ROR1 antibody recognizes an epitope located within the immunoglobulin (Ig) domain, the Frizzled domain, or the Kringle domain of human ROR1; and an excipient and/or a delivery vehicle. In some embodiments, the payload comprises an auristatin derivative, maytansine, a maytansinoid, a taxane, a calicheamicin, cemadotin, a duocarmycin, a pyrrolobenzodiazepine (PBD), a tubulysin, or a combination thereof. In some embodiments, the auristatin derivative is monomethyl auristatin E (MMAE). In some embodiments, the auristatin derivative is monomethyl auristatin F (MMAF). In some embodiments, the maytansinoid comprises DM1 (mertansine) or DM4. In some embodiments, the pyrrolobenzodiazepine is a pyrrolobenzodiazepine dimer. In some embodiments, the anti-ROR1 antibody-payload conjugate further comprises a linker moiety that attaches the anti-ROR1 antibody to the payload. In some embodiments, the linker moiety comprises a homobifunctional linker or a heterobifunctional linker. In some embodiments, the linker moiety comprises a cleavable linker. In some embodiments, the linker moiety comprises a non-cleavable linker. In some embodiments, the linker moiety comprises a valine-citrulline moiety. In some embodiments, the linker moiety further comprises p-aminobenzoic acid. In some embodiments, the anti-ROR1 antibody further comprises a formylglycine residue generated by a formylglycine-generating enzyme. In some embodiments, the payload is conjugated to the anti-ROR1 antibody at the formylglycine site. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 3, (ii) a variable heavy (VH) CDR2 that has an amino acid sequence of

SEQ ID NO: 4, and (iii) a variable heavy (VH) CDR3 that has an amino acid sequence of SEQ ID NO: 5, and wherein the light chain variable region comprises (iv) a variable light (VL) CDR 1 that has an amino acid sequence of SEQ ID NO: 6, (v) a variable light (VL) CDR 2 that has an amino acid sequence of SEQ ID NO: 7, and (vi) a variable light (VL) CDR 3 that has an amino acid sequence of SEQ ID NO: 8. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 20, (ii) a variable heavy (VH) CDR2 that has an amino acid sequence of SEQ ID NO: 21, and (iii) a variable heavy (VH) CDR3 that has an amino acid sequence of SEQ ID NO: 22, and wherein the light chain variable region comprises (iv) a variable light (VL) CDR 1 that has an amino acid sequence of SEQ ID NO: 23, (v) a variable light (VL) CDR 2 that has an amino acid sequence of SEQ ID NO: 24, and (vi) a variable light (VL) CDR 3 that has an amino acid sequence of SEQ ID NO: 25. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 30, (ii) a variable heavy (VH) CDR2 that has an amino acid sequence of SEQ ID NO: 31, and (iii) a variable heavy (VH) CDR3 that has an amino acid sequence of SEQ ID NO: 32, and wherein the light chain variable region comprises (iv) a variable light (VL) CDR 1 that has an amino acid sequence of SEQ ID NO: 33, (v) a variable light (VL) CDR 2 that has an amino acid sequence of SEQ ID NO: 34, and (vi) a variable light (VL) CDR 3 that has an amino acid sequence of SEQ ID NO: 35. In some embodiments, the anti-ROR1 antibody comprises a heavy chain variable region comprising three complementarity determining regions (CDRs) and a light chain variable region comprising three CDRs, wherein the heavy chain variable region comprises (i) a variable heavy (VH) CDR1 that has an amino acid sequence of SEQ ID NO: 38, (ii) a variable heavy (VH) CDR2 that has an amino acid sequence of SEQ ID NO: 39, and; (iii) a variable heavy (VH) CDR3 that has an amino acid sequence of SEQ ID NO: 40, and wherein the light chain variable region comprises (iv) a variable light (VL) CDR 1 that has an amino acid sequence of SEQ ID NO: 41, (v) a variable light (VL) CDR 2 that has an amino acid sequence of SEQ ID NO: 42, and (vi) a variable light (VL) CDR 3 that has an amino acid sequence of SEQ ID NO: 43. In some embodiments, the anti-ROR1 antibody comprises a heavy chain variable region having at least 80%, 85%, 90%, 95%, or 99% sequence identity to SEQ ID NO: 1, 9, or 13 and a light chain variable region having at least 80%, 85%, 90%, 95% or 99% sequence identity to SEQ ID NO: 2, 10 or 14. In some embodiments, the anti-ROR1 antibody comprises a heavy chain variable region of SEQ ID NO: 1 and a light chain variable region of SEQ ID NO: 2. In some embodiments, the anti-ROR1 antibody comprises a heavy chain variable region of SEQ ID NO: 9 and a light chain variable region of SEQ ID NO: 10. In some embodiments, the anti-ROR1 antibody comprises a heavy chain variable region of SEQ ID NO: 13 and a light chain variable region of SEQ ID NO: 14. In some embodiments, the anti-ROR1 antibody comprises a heavy chain variable region having at least 80%, 85%, 90%, 95%, or 99% sequence identity to SEQ ID NO: 17 or 18 and a light chain variable region having at least 80%, 85%, 90%, 95%, or 99% sequence identity to SEQ ID NO: 19. In some embodiments, the anti-ROR1 antibody comprises a heavy chain variable region of SEQ ID NO: 17 and a light chain variable region of SEQ ID NO: 19. In some embodiments, the anti-ROR1 antibody comprises a heavy chain variable region of SEQ ID NO: 18 and a light chain variable region of SEQ ID NO: 19. In some embodiments, the anti-ROR1 antibody comprises a heavy chain variable region having at least 80%, 85%, 90%, 95%, or 99% sequence identity to SEQ ID NO: 28 and a light chain variable region having at least 80%, 85%, 90%, 95%, or 99% sequence identity to SEQ ID NO: 29. In some embodiments, the anti-ROR1 antibody comprises a heavy chain variable region of SEQ ID NO: 28 and a light chain variable region of SEQ ID NO: 29. In some embodiments, the anti-ROR1 antibody comprises a heavy chain variable region having at least 80%, 85%, 90%, 95%, or 99% sequence identity to SEQ ID NO: 36 and a light chain variable region having at least 80%, 85%, 90%, 95%, or 99% sequence identity to SEQ ID NO: 37. In some embodiments, the anti-ROR1 antibody comprises a heavy chain variable region of SEQ ID NO: 36 and a light chain variable region of SEQ ID NO: 37. In some embodiments, the anti-ROR1 antibody-payload conjugate comprises about 2, 3, 4, 5, 6, or more payloads. In some embodiments, the anti-ROR1 antibody-payload conjugate comprises about 4 payloads. In some embodiments, the payloads are the same. In some embodiments, the payloads are different. In some embodiments, the anti-ROR1 antibody-payload conjugate is formulated for parenteral administration. In some embodiments, the anti-ROR1 antibody-payload conjugate is formulated for intranasal administration.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 embodies the domains and regions present in a human ROR1 protein. Extracellular domains include the immunoglobulin (Ig) domain, frizzled domain, and Kringle domain. The tyrosine kinase domain is intracellular, as is the serine/threonine rich region and proline rich region.

FIG. 2 illustrates ROR1 mRNA expression in 640 cell lines.

FIG. 3 illustrates an 8% non-reducing SDS-PAGE analysis of expressing supernatants.

FIG. 4A-FIG. 4F illustrate SEC-HPLC profiles of anti-ROR1 antibodies. FIG. 4A illustrates the SEC-HPLC profile of m2A2. FIG. 4B illustrates the SEC-HPLC profile of c2A2. FIG. 4C illustrates the SEC-HPLC profile of cR11. FIG. 4D illustrates the SEC-HPLC profile of cY31. FIG. 4E illustrates the SEC-HPLC profile of cD10. FIG. 4F illustrates the SEC-HPLC profile of cR12. m=murine, c=chimeric

FIG. 5A-FIG. 5L illustrate hydrophobic interaction chromatogram (HIC) profiles. FIG. 5A illustrates the HIC profile of the h2A2 conjugate. FIG. 5B illustrates the HIC profile of the h2A2 monoclonal antibody. FIG. 5C illustrates the HIC profile of the h2A2m conjugate. FIG. 5D illustrates the HIC profile of the h2A2 monoclonal antibody. FIG. 5E illustrates the HIC profile of the m2A2 conjugate. FIG. 5F illustrates the HIC profile of the m2A2 monoclonal antibody. FIG. 5G illustrates the HIC profile of the R12 conjugate. FIG. 5H illustrates the HIC profile of the R12 monoclonal antibody. FIG. 5I illustrates the HIC profile of the Y31 conjugate. FIG. 5J illustrates the HIC profile of the Y31 monoclonal antibody. FIG. 5K illustrates the HIC profile of the R11 conjugate. FIG. 5L illustrates the HIC profile of the R11 monoclonal antibody.

FIG. 6 illustrate FITC signal distribution of the control at 37 deg, 2.5 hr.

FIG. 7A-FIG. 7C illustrate FITC signal distribution of untagged 2A2. FIG. 7A illustrates FITC signal distribution of untagged 2A2 at T0 (4 deg). FIG. 7B illustrates FITC signal distribution of untagged 2A2 at T2 (37 deg, 2.5 hr). FIG. 7C illustrates FITC signal distribution of the control (secondary) at T2 (FIG. 6), untagged 2A2 at T0, and untagged 2A2 at T2, illustrating an internalization of the untagged 2A2 of 17.8%.

FIG. 8A-FIG. 8C illustrate FITC signal distribution of CH1-tagged 2A2. FIG. 8A illustrates FITC signal distribution of CH1-tagged 2A2 at TO (4 deg). FIG. 8B illustrates FITC signal distribution of CH1-tagged 2A2 at T2 (37 deg, 2.5 hr). FIG. 8C illustrates FITC signal distribution of the control (secondary) at T2 (FIG. 6), CH1-tagged 2A2 at T0, and CH1-tagged 2A2 at T2, illustrating an internalization of the CH1-tagged 2A2 of 28.7%.

FIG. 9A-FIG. 9C illustrate FITC signal distribution of CT-tagged 2A2. FIG. 9A illustrates FITC signal distribution of C-terminal (CT)-tagged 2A2 at TO (4 deg). FIG. 9B illustrates FITC signal distribution of C-terminal (CT)-tagged 2A2 at T2 (37 deg, 2.5 hr). FIG. 9C illustrates FITC signal distribution of the control (secondary) at T2 (FIG. 6), C-terminal (CT)-tagged 2A2 at T0, and C-terminal (CT)-tagged 2A2 at T2, illustrating an internalization of the C-terminal (CT)-tagged 2A2 of 24.2%.

FIG. 10A-FIG. 10C illustrate FITC signal distribution of CH1/CT double-tagged 2A2. FIG. 10A illustrates FITC signal distribution of CH1/CT double-tagged 2A2 at T0 (4 deg). FIG. 10B illustrates FITC signal distribution of CH1/CT double-tagged 2A2 at T2 (37 deg, 2.5 hr). FIG. 10C illustrates FITC signal distribution of the control (secondary) at T2 (FIG. 6), CH1/CT double-tagged 2A2 at T0, and CH1/CT double-tagged 2A2 at T2, illustrating an internalization of the CH1/CT double-tagged 2A2 of 40.2%.

FIG. 11A-FIG. 11B illustrate determination of IC50 values. FIG. 11A illustrates determination of the IC50 value of chimeric 2A2. FIG. 11B illustrates determination of the IC50 value of chimeric R11.

FIG. 12A-FIG. 12M illustrate ROR1 expression in patient-derived tumor xenographs. FIG. 12A illustrates ROR1 expression in the NCI-H2228 control. FIG. 12B illustrates ROR1 expression in the MHCC97H control. FIG. 12C illustrates ROR1 expression in the MKN45 control. FIG. 12D illustrates ROR1 expression in the Daudi control. FIG. 12E illustrates ROR1 expression on TMA-set#13, gastric cancer PDX in GA0087 P6. FIG. 12F illustrates ROR1 expression on TMA-set#13, gastric cancer PDX in GA0095 P6. FIG. 12G illustrates ROR1 expression on TMA-set#13, gastric cancer PDX in GA0098 P4. FIG. 12H illustrates ROR1 expression on TMA-set#13, liver cancer PDX in LI0612 P6. FIG. 12I illustrates ROR1 expression on TMA-set#13, liver cancer PDX in LI1098 P6. FIG. 12J illustrates ROR1 expression on TMA-set#13, liver cancer PDX in LI6662 P4. FIG. 12K illustrates ROR1 expression on TMA-set#13, lung cancer PDX in LU0330 P5. FIG. 12L illustrates ROR1 expression on TMA-set#13, lung cancer PDX in LU0858 P7. FIG. 12M illustrates ROR1 expression on TMA-set#13, lung cancer PDX in LU3075 P9.

FIG. 13 illustrates body weight in tumor bearing mice. Data expressed as mean±SEM.

FIG. 14 illustrates body weight changes in tumor bearing mice. Data expressed as mean ±SEM.

FIG. 15 illustrates tumor growth curves. Data expressed as mean±SEM.

FIG. 16A-FIG. 16B illustrate staining of tumor cross-sections by immunohistochemistry (IHC) at two different magnification levels. FIG. 16A illustrates IHC staining of Group 1 mice. FIG. 16B illustrates IHC staining of Group 2 mice.

FIG. 17 illustrates mean tumor volume of tumors treated with chimeric 2A2-vcMMAE, chimeric R11-vcMMAE, chimeric 2A2-DM1, and chimeric 2A2-Duocarmycin. Arrows represent application of a 5 mg/kg dose on days 4, 8, 12, and 16.

FIG. 18 illustrates percent inhibition of tumor volume of tumors treated with chimeric 2A2-vcMMAE, chimeric R11-vcMMAE, chimeric 2A2-DM1, and chimeric 2A2-Duocarmycin.

FIG. 19 illustrates change in tumor volume over days post injection in Jeko-1 Xenograft mice treated with chimeric 2A2 conjugates.

### DETAILED DESCRIPTION OF THE INVENTION

Receptor tyrosine kinase-like orphan receptor 1 (ROR1) is present during normal embryonic and fetal development, but absent or low in most mature tissue. In some instances, high expression of ROR1 has been found in different types of blood and solid malignancies. Indeed, studies have shown expression of ROR1 on cell surface of malignant cells such as B-cells of chronic lymphocytic leukemia (CLL) and mantle cell lymphoma (MCL).

Disclosed herein, in some embodiments, are anti-ROR1 antibody-payload conjugates and methods of use thereof. In some embodiments, included herein is a method of treating a subject having cancer, which comprises administering to the subject a therapeutically effective amount of a pharmaceutical composition comprising an anti-ROR1 antibody-payload conjugate wherein the subject has bladder cancer, breast cancer, colorectal cancer, liver cancer, lung cancer, pancreatic cancer, renal cell carcinoma, stomach cancer, adrenal cancer, skin cancer, prostate cancer, B-cell lymphoma or acute lymphoblastic leukemia.

In some embodiments, also included herein is a method of treating a subject having liver cancer, comprising: administering to the subject having liver cancer a therapeutically effective amount of a pharmaceutical composition comprising an anti-ROR1 antibody-payload conjugate wherein the anti-ROR1 antibody recognizes an epitope located within the immunoglobulin (Ig) domain, the Frizzled domain, or the Kringle domain of human ROR1.

In some embodiments, additionally included herein is an anti-ROR1 antibody comprising a heavy chain variable region having at least 80%, 85%, 90%, 95%, or 99% sequence identity to SEQ ID NO: 15 and a light chain variable region having at least 80%, 85%, 90%, 95%, or 99% sequence identity to SEQ ID NO: 16, a nucleic acid polymer encoding the anti-ROR1 antibody, and a pharmaceutical composition comprising the same.

### Certain Terminologies

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms refer to human or animal subjects. Animal subjects include, but are not limited to, animal models, such as, mammalian models of conditions or disorders associated with elevated ROR1 expression such as B-CLL, MCL, Burkett's lymphoma, renal cell carcinoma, colon cancer, (e.g., colon adenocarcinoma), and breast cancer (e.g., breast adenocarcinoma).

“Treating” or “treatment” of a state, disorder or condition (e.g., cancer) includes: (1) preventing or delaying the appearance of clinical or sub-clinical symptoms of the disorder developing in a human that is afflicted with or pre-disposed to the disorder but does not yet experience or display clinical or subclinical symptoms of the disorder; and/or (2) inhibiting the disorder, including arresting, reducing or delaying the clinical manifestation of the disorder or at least one clinical or sub-clinical symptom thereof; and/or (3) relieving the disorder, e.g., causing regression of the disorder or at least one of its clinical or sub-clinical symptoms; and/or (4) causing a decrease in the severity of one or more symptoms of the disorder. The benefit to a subject to be treated is either statistically significant or at least perceptible to the patient or to the physician.

The term “antibody” as used herein refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and portions thereof; including, for example, an immunoglobulin molecule, a polyclonal antibody, a monoclonal antibody, a recombinant antibody, a chimeric antibody, a humanized antibody, a CDR-grafted antibody, F(ab)₂, Fv, scFv, IgGΔCH₂, F(ab′)2, scFv2CH₃, F(ab), VL, VH, scFv4, scFv3, scFv2, dsFv, Fv, scFv-Fc, (scFv)2, a disulfide linked Fv, a single domain antibody (dAb), a diabody, a multispecific antibody, a dual specific antibody, an anti-idiotypic antibody, a bispecific antibody, any isotype (including, without limitation IgA, IgD, IgE, IgG, or IgM) a modified antibody, and a synthetic antibody (including, without limitation non-depleting IgG antibodies, T-bodies, or other Fc or Fab variants of antibodies). Each heavy chain is composed of a variable region of said heavy chain (abbreviated here as HCVR or VH) and a constant region of said heavy chain. Each light chain is composed of a variable region of said light chain (abbreviated here as LCVR or VL) and a constant region of said light chain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4.

As used herein, the term “CDR” refers to the complementarity determining region within antibody variable sequences. There are three CDRs in each of the variable regions of the heavy chain and of the light chain, which are designated CDR1, CDR2 and CDR3, for each of the variable regions. The exact boundaries of these CDRs have been defined differently according to different systems, including those described by: Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745.” (“Contact” numbering scheme), Lefranc M P et al., “IMGT unique numbering for immunoglobulin and T cell receptor variable domains and Ig superfamily V-like domains,” Dev Comp Immunol, 2003 Jan;27(1):55-77 (“IMGT” numbering scheme), and Honegger A and Plückthun A, “Yet another numbering scheme for immunoglobulin variable domains: an automatic modeling and analysis tool,” J Mol Biol, 2001 Jun. 8;309(3):657-70, (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The EU index or EU index as in Kabat or EU numbering scheme refers to the numbering of the EU antibody (Edelman et al., 1969, Proc Natl Acad Sci USA 63:78-85. In some embodiments, the methods used herein utilize CDRs defined according to any of these systems. In some embodiments, the methods used herein utilize CDRs defined according to the Kabat system.

### Antibody-Payload Conjugates

Disclosed herein, in certain embodiments, are compositions comprising an anti-ROR1 antibody-payload conjugate. In some embodiments, the antibody-payload conjugate is an antibody-drug conjugate (ADC). In some instances, an antibody-payload conjugate comprises a monoclonal antibody (mAb), which selectively binds cancer-specific antigens, a cytotoxic drug payload, which induces cell death, and a small molecule linker, which connects the antibody to the payload. By coupling the pharmacokinetic (PK) profile and targetability of monoclonal antibodies with the potent cytotoxicity of small molecule drugs (i.e. the payload), dose-limiting toxicities can be minimized while desired therapeutic effects can be maximized. A variety of linkers are available to conjugate the payload to the antibody and can affect characteristics of the antibody-payload conjugate, such as serum stability, mechanism of release, and drug-to-antibody ratio (DAR), among others.

### Anti-ROR1 Antibody

Receptor tyrosine kinase-like orphan receptor 1 (ROR1) is a conserved embryonic protein whose expression becomes progressively reduced during embryonic development in mammals. The intact protein, including its extracellular domain, appears to be expressed at low levels in normal, adult mammalian tissues. In some instances, studies have not identified significant expression of ROR1 on the cell surface of normal adult human tissues, including normal B cells. In some cases, ROR1 is expressed on the cell surface of malignant B-cells, for example, B-cell chronic lymphocytic leukemia (B-CLL) and mantle cell lymphoma (MCL). In additional cases, it has also been reported that ROR1 is expressed in cancer cell lines such as, for example, Burkett's lymphoma, renal cell carcinoma, colon cancer, breast cancer, bladder cancer, breast cancer, colorectal cancer, liver cancer, pancreatic cancer, stomach cancer, B-cell lymphoma or acute lymphoblastic leukemia.

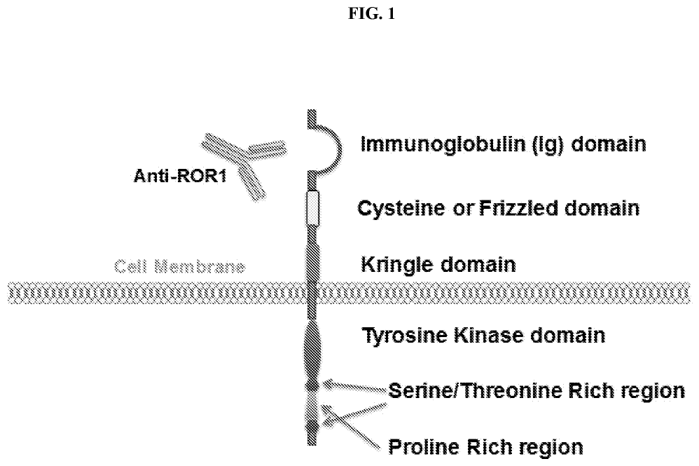

In some embodiments, the anti-ROR1 antibody binds to a ROR1 polypeptide or functional fragment thereof. In some embodiments, the ROR1 polypeptide or functional fragment thereof is a human ROR1 polypeptide or functional fragment thereof. In some embodiments, the ROR1 protein has an amino acid sequence of SEQ ID NO: 44. In some embodiments, the anti-ROR1 antibody binds to one or more domains of ROR1, for example, to the Immunoglobulin (Ig) domain, cysteine or Frizzled domain, Kringle domain, or a combination thereof (FIG. 1). In some embodiments, the anti-ROR1 antibody binds to the Immunoglobulin (Ig) domain of ROR1. In some embodiments, the anti-ROR1 antibody binds to the cysteine or Frizzled domain of ROR1. In some embodiments, the anti-ROR1 antibody binds to the Kringle domain of ROR1. In some embodiments, the anti-ROR1 antibody binds to one or more domains of ROR1 comprising SEQ ID NO: 45, SEQ ID NO: 46 or SEQ ID NO: 47. In some embodiments, the anti-ROR1 antibody binds to two or more domains of ROR1, selected from the Immunoglobulin (Ig) domain, the Frizzled domain, and the Kringle domain. In some embodiments, anti-ROR1 antibody binds to a junction between two ROR1 domains. In some embodiments, the anti-ROR1 antibody binds to the junction between the Immunoglobulin domain and the Frizzled domain. In some embodiments, the anti-ROR1 antibody binds to the junction between the Frizzled domain and the Kringle domain.

In some embodiments, an anti-ROR1 antibody described herein comprises a variable heavy (VH) chain comprising three complementarity determining regions (CDRs) selected from SEQ ID NOs: 3-5, 20-22, 30-32 and 38-40. In some cases, an anti-ROR1 antibody described herein comprises a variable heavy (VH) chain comprising three complementarity determining regions (CDRs) in which CDR1 comprises SEQ ID NO: 3, CDR2 comprises SEQ IDN O: 4, and CDR3 comprises SEQ ID NO: 5. In some cases, an anti-ROR1 antibody described herein comprises a variable heavy (VH) chain comprising three complementarity determining regions (CDRs) in which CDR1 comprises SEQ ID NO: 20, CDR2 comprises SEQ ID NO: 21, and CDR3 comprises SEQ ID NO:22. In some cases, an anti-ROR1 antibody described herein comprises a variable heavy (VH) chain comprising three complementarity determining regions (CDRs) in which CDR1 comprises SEQ ID NO: 30, CDR2 comprises SEQ ID NO: 31, and CDR3 comprises SEQ ID NO: 32. In some cases, an anti-ROR1 antibody described herein comprises a variable heavy (VH) chain comprising three complementarity determining regions (CDRs) in which CDR1 comprises SEQ ID NO: 38, CDR2 comprises SEQ ID NO: 39, and CDR3 comprises SEQ ID NO: 40.

In some embodiments, an anti-ROR1 antibody described herein further comprises a variable light (VL) chain complementarity determining regions (CDRs) selected from SEQ ID NOs: 6-8, 23-25, 33-35 and 41-43. In some cases, an anti-ROR1 antibody described herein comprises a variable light (VL) chain comprising three complementarity determining regions (CDRs) in which CDR1 comprises SEQ ID NO: 6, CDR2 comprises SEQ ID NO: 7, and CDR3 comprises SEQ ID NO: 8. In some cases, an anti-ROR1 antibody described herein comprises a variable light (VL) chain comprising three complementarity determining regions (CDRs) in which CDR1 comprises SEQ ID NO: 23, CDR2 comprises SEQ ID NO: 24, and CDR3 comprises SEQ ID NO: 25. In some cases, an anti-ROR1 antibody described herein comprises a variable light (VL) chain comprising three complementarity determining regions (CDRs) in which CDR1 comprises SEQ ID NO: 33, CDR2 comprises SEQ ID NO: 34, and CDR3 comprises SEQ ID NO: 35. In some cases, an anti-ROR1 antibody described herein comprises a variable light (VL) chain comprising three complementarity determining regions (CDRs) in which CDR1 comprises SEQ ID NO: 41, CDR2 comprises SEQ ID NO: 42, and CDR3 comprises SEQ ID NO: 43.

### Antibody 2A2 and Variants

In some embodiments, an anti-ROR1 antibody described herein comprises a heavy chain variable region having at least 80%, 85%, 90%, 95%, or 99% sequence identity to SEQ ID NO: 1, 9 or 13. In some embodiments, an anti-ROR1 antibody described herein comprises a heavy chain variable region having at least 80%, 85%, 90%, 95%, or 99% sequence identity to SEQ ID NO: 1. In some embodiments, an anti-ROR1 antibody described herein comprises a heavy chain variable region having at least 80% sequence identity to SEQ ID NO: 1. In some embodiments, an anti-ROR1 antibody described herein comprises a heavy chain variable region having at least 85% sequence identity to SEQ ID NO: 1. In some embodiments, an anti-ROR1 antibody described herein comprises a heavy chain variable region having at least 90% sequence identity to SEQ ID NO: 1. In some embodiments, an anti-ROR1 antibody described herein comprises a heavy chain variable region having at least 95% sequence identity to SEQ ID NO: 1. In some embodiments, an anti-ROR1 antibody described herein comprises a heavy chain variable region having at least 99% sequence identity to SEQ ID NO: 1.

In some embodiments, an anti-ROR1 antibody described herein comprises a heavy chain variable region having at least 80%, 85%, 90%, 95%, or 99% sequence identity to SEQ ID NO: 9. In some embodiments, an anti-ROR1 antibody described herein comprises a heavy chain variable region having at least 80% sequence identity to SEQ ID NO: 9. In some embodiments, an anti-ROR1 antibody described herein comprises a heavy chain variable region having at least 85% sequence identity to SEQ ID NO: 9. In some embodiments, an anti-ROR1 antibody described herein comprises a heavy chain variable region having at least 90% sequence identity to SEQ ID NO: 9. In some embodiments, an anti-ROR1 antibody described herein comprises a heavy chain variable region having at least 95% sequence identity to SEQ ID NO: 9. In some embodiments, an anti-ROR1 antibody described herein comprises a heavy chain variable region having at least 99% sequence identity to SEQ ID NO: 9.

In some embodiments, an anti-ROR1 antibody described herein comprises a heavy chain variable region having at least 80%, 85%, 90%, 95%, or 99% sequence identity to SEQ ID NO: 13. In some embodiments, an anti-ROR1 antibody described herein comprises a heavy chain variable region having at least 80% sequence identity to SEQ ID NO: 13. In some embodiments, an anti-ROR1 antibody described herein comprises a heavy chain variable region having at least 85% sequence identity to SEQ ID NO: 13. In some embodiments, an anti-ROR1 antibody described herein comprises a heavy chain variable region having at least 90% sequence identity to SEQ ID NO: 11. In some embodiments, an anti-ROR1 antibody described herein comprises a heavy chain variable region having at least 95% sequence identity to SEQ ID NO: 13. In some embodiments, an anti-ROR1 antibody described herein comprises a heavy chain variable region having at least 99% sequence identity to SEQ ID NO: 13.

In some embodiments, an anti-ROR1 antibody described herein comprises a light chain variable region having at least 80%, 85%, 90%, 95%, or 99% sequence identity to SEQ ID NO: 2, 10, or 14. In some embodiments, an anti-ROR1 antibody described herein comprises a light chain variable region having at least 80%, 85%, 90%, 95%, or 99% sequence identity to SEQ ID NO: 2. In some embodiments, an anti-ROR1 antibody described herein comprises a light chain variable region having at least 80% sequence identity to SEQ ID NO: 2. In some embodiments, an anti-ROR1 antibody described herein comprises a light chain variable region having at least 85% sequence identity to SEQ ID NO: 2. In some embodiments, an anti-ROR1 antibody described herein comprises a light chain variable region having at least 90% sequence identity to SEQ ID NO: 2. In some embodiments, an anti-ROR1 antibody described herein comprises a light chain variable region having at least 95% sequence identity to SEQ ID NO: 2. In some embodiments, an anti-ROR1 antibody described herein comprises a light chain variable region having at least 99% sequence identity to SEQ ID NO: 2.

In some embodiments, an anti-ROR1 antibody described herein comprises a light chain variable region having at least 80%, 85%, 90%, 95%, or 99% sequence identity to SEQ ID NO: 10. In some embodiments, an anti-ROR1 antibody described herein comprises a light chain variable region having at least 80% sequence identity to SEQ ID NO: 10. In some embodiments, an anti-ROR1 antibody described herein comprises a light chain variable region having at least 85% sequence identity to SEQ ID NO: 10. In some embodiments, an anti-ROR1 antibody described herein comprises a light chain variable region having at least 90% sequence identity to SEQ ID NO: 10. In some embodiments, an anti-ROR1 antibody described herein comprises a light chain variable region having at least 95% sequence identity to SEQ ID NO: 10. In some embodiments, an anti-ROR1 antibody described herein comprises a light chain variable region having at least 99% sequence identity to SEQ ID NO: 10.

In some embodiments, an anti-ROR1 antibody described herein comprises a light chain variable region having at least 80%, 85%, 90%, 95%, or 99% sequence identity to SEQ ID NO: 14. In some embodiments, an anti-ROR1 antibody described herein comprises a light chain variable region having at least 80% sequence identity to SEQ ID NO: 14. In some embodiments, an anti-ROR1 antibody described herein comprises a light chain variable region having at least 85% sequence identity to SEQ ID NO: 14. In some embodiments, an anti-ROR1 antibody described herein comprises a light chain variable region having at least 90% sequence identity to SEQ ID NO: 14. In some embodiments, an anti-ROR1 antibody described herein comprises a light chain variable region having at least 95% sequence identity to SEQ ID NO: 14. In some embodiments, an anti-ROR1 antibody described herein comprises a light chain variable region having at least 99% sequence identity to SEQ ID NO: 14.

In some embodiments, an anti-ROR1 antibody described herein comprises a heavy chain variable region of SEQ ID NO: 1 and a light chain variable region of SEQ ID NO: 2.

In some embodiments, an anti-ROR1 antibody described herein comprises a heavy chain variable region of SEQ ID NO: 9 and a light chain variable region of SEQ ID NO: 10.

In some embodiments, an anti-ROR1 antibody described herein comprises a heavy chain variable region of SEQ ID NO: 13 and a light chain variable region of SEQ ID NO: 14.

In some embodiments, an anti-ROR1 antibody described herein comprises a heavy chain variable region comprising three complementarity determining regions and a light chain variable region comprising three CDRs, in which the heavy chain variable region comprises a variable heavy chain (VH) CDR1 that has an amino acid sequence of SEQ ID NO: 3, a variable heavy chain (VH) CDR2 that has an amino acid sequence of SEQ ID NO: 4, a variable heavy chain (VH) CDR3 that has an amino acid sequence of SEQ ID NO: 5, and the light chain variable region comprises a variable light chain (VL) CDR1 that has an amino acid sequence of SEQ ID NO: 6, a variable light (VL) CDR2 that has an amino acid sequence of SEQ ID NO: 7, and a variable light (VL) CDR3 that has an amino acid sequence of SEQ ID NO:8. In some embodiments, an anti-ROR1 antibody described herein is 2A2.

### Antibody R11 and Variants

In some embodiments, an anti-ROR1 antibody described herein comprises a heavy chain variable region having at least 80%, 85%, 90%, 95%, or 99% sequence identity to SEQ ID NO: 17. In some embodiments, an anti-ROR1 antibody described herein comprises a heavy chain variable region having at least 80% sequence identity to SEQ ID NO: 17. In some embodiments, an anti-ROR1 antibody described herein comprises a heavy chain variable region having at least 85% sequence identity to SEQ ID NO: 17. In some embodiments, an anti-ROR1 antibody described herein comprises a heavy chain variable region having at least 90% sequence identity to SEQ ID NO: 17. In some embodiments, an anti-ROR1 antibody described herein comprises a heavy chain variable region having at least 95% sequence identity to SEQ ID NO: 17. In some embodiments, an anti-ROR1 antibody described herein comprises a heavy chain variable region having at least 99% sequence identity to SEQ ID NO: 17.

In some embodiments, an anti-ROR1 antibody described herein comprises a heavy chain variable region having at least 80%, 85%, 90%, 95%, or 99% sequence identity to SEQ ID NO: 18. In some embodiments, an anti-ROR1 antibody described herein comprises a heavy chain variable region having at least 80% sequence identity to SEQ ID NO: 18. In some embodiments, an anti-ROR1 antibody described herein comprises a heavy chain variable region having at least 85% sequence identity to SEQ ID NO: 18. In some embodiments, an anti-ROR1 antibody described herein comprises a heavy chain variable region having at least 90% sequence identity to SEQ ID NO: 18. In some embodiments, an anti-ROR1 antibody described herein comprises a heavy chain variable region having at least 95% sequence identity to SEQ ID NO: 18. In some embodiments, an anti-ROR1 antibody described herein comprises a heavy chain variable region having at least 99% sequence identity to SEQ ID NO: 18.

In some embodiments, an anti-ROR1 antibody described herein comprises a light chain variable region having at least 80%, 85%, 90%, 95%, or 99% sequence identity to SEQ ID NO: 19. In some embodiments, an anti-ROR1 antibody described herein comprises a light chain variable region having at least 80% sequence identity to SEQ ID NO: 19. In some embodiments, an anti-ROR1 antibody described herein comprises a light chain variable region having at least 85% sequence identity to SEQ ID NO: 19. In some embodiments, an anti-ROR1 antibody described herein comprises a light chain variable region having at least 90% sequence identity to SEQ ID NO: 19. In some embodiments, an anti-ROR1 antibody described herein comprises a light chain variable region having at least 95% sequence identity to SEQ ID NO: 19. In some embodiments, an anti-ROR1 antibody described herein comprises a light chain variable region having at least 99% sequence identity to SEQ ID NO: 19.

In some embodiments, an anti-ROR1 antibody described herein comprises a heavy chain variable region of SEQ ID NO: 17 and a light chain variable region of SEQ ID NO: 19.

In some embodiments, an anti-ROR1 antibody described herein comprises a heavy chain variable region of SEQ ID NO: 18 and a light chain variable region of SEQ ID NO: 19.

In some embodiments, an anti-ROR1 antibody described herein comprises a heavy chain variable region comprising three complementarity determining regions and a light chain variable region comprising three CDRs, in which the heavy chain variable region comprises a variable heavy chain (VH) CDR1 that has an amino acid sequence of SEQ ID NO: 20, a variable heavy chain (VH) CDR2 that has an amino acid sequence of SEQ ID NO: 21, a variable heavy chain (VH) CDR3 that has an amino acid sequence of SEQ ID NO: 22, and the light chain variable region comprises a variable light chain (VL) CDR1 that has an amino acid sequence of SEQ ID NO: 23, a variable light (VL) CDR2 that has an amino acid sequence of SEQ ID NO: 24, and a variable light (VL) CDR3 that has an amino acid sequence of SEQ ID NO: 25. In some embodiments, an anti-ROR1 antibody described herein is R11.

### Antibody R12 and Variants

In some embodiments, an anti-ROR1 antibody described herein comprises a heavy chain variable region having at least 80%, 85%, 90%, 95%, or 99% sequence identity to SEQ ID NO: 28. In some embodiments, an anti-ROR1 antibody described herein comprises a heavy chain variable region having at least 80% sequence identity to SEQ ID NO: 28. In some embodiments, an anti-ROR1 antibody described herein comprises a heavy chain variable region having at least 85% sequence identity to SEQ ID NO: 28. In some embodiments, an anti-ROR1 antibody described herein comprises a heavy chain variable region having at least 90% sequence identity to SEQ ID NO: 28. In some embodiments, an anti-ROR1 antibody described herein comprises a heavy chain variable region having at least 95% sequence identity to SEQ ID NO: 28. In some embodiments, an anti-ROR1 antibody described herein comprises a heavy chain variable region having at least 99% sequence identity to SEQ ID NO: 28.

In some embodiments, an anti-ROR1 antibody described herein comprises a light chain variable region having at least 80%, 85%, 90%, 95%, or 99% sequence identity to SEQ ID NO: 29. In some embodiments, an anti-ROR1 antibody described herein comprises a light chain variable region having at least 80% sequence identity to SEQ ID NO: 29. In some embodiments, an anti-ROR1 antibody described herein comprises a light chain variable region having at least 85% sequence identity to SEQ ID NO: 29. In some embodiments, an anti-ROR1 antibody described herein comprises a light chain variable region having at least 90% sequence identity to SEQ ID NO: 29. In some embodiments, an anti-ROR1 antibody described herein comprises a light chain variable region having at least 95% sequence identity to SEQ ID NO: 29. In some embodiments, an anti-ROR1 antibody described herein comprises a light chain variable region having at least 99% sequence identity to SEQ ID NO: 29.

In some embodiments, an anti-ROR1 antibody described herein comprises a heavy chain variable region of SEQ ID NO: 28 and a light chain variable region of SEQ ID NO: 29.

In some embodiments, an anti-ROR1 antibody described herein comprises a heavy chain variable region comprising three complementarity determining regions and a light chain variable region comprising three CDRs, wherein the heavy chain variable region comprises a variable heavy chain (VH) CDR1 that has an amino acid sequence of SEQ ID NO: 30, a variable heavy chain (VH) CDR2 that has an amino acid sequence of SEQ ID NO: 31, a variable heavy chain (VH) CDR3 that has an amino acid sequence of SEQ ID NO: 32, and wherein the light chain variable region comprises a variable light chain (VL) CDR1 that has an amino acid sequence of SEQ ID NO: 33, a variable light (VL) CDR2 that has an amino acid sequence of SEQ ID NO: 34, and a variable light (VL) CDR3 that has an amino acid sequence of SEQ ID NO:35. In some embodiments, an anti-ROR1 antibody described herein is R12.

### Antibody Y31 and Variants

In some embodiments, an anti-ROR1 antibody described herein comprises a heavy chain variable region having at least 80%, 85%, 90%, 95%, or 99% sequence identity to SEQ ID NO: 36. In some embodiments, an anti-ROR1 antibody described herein comprises a heavy chain variable region having at least 80% sequence identity to SEQ ID NO: 36. In some embodiments, an anti-ROR1 antibody described herein comprises a heavy chain variable region having at least 85% sequence identity to SEQ ID NO: 36. In some embodiments, an anti-ROR1 antibody described herein comprises a heavy chain variable region having at least 90% sequence identity to SEQ ID NO: 36. In some embodiments, an anti-ROR1 antibody described herein comprises a heavy chain variable region having at least 95% sequence identity to SEQ ID NO: 36. In some embodiments, an anti-ROR1 antibody described herein comprises a heavy chain variable region having at least 99% sequence identity to SEQ ID NO: 36.

In some embodiments, an anti-ROR1 antibody described herein comprises a light chain variable region having at least 80%, 85%, 90%, 95%, or 99% sequence identity to SEQ ID NO: 37. In some embodiments, an anti-ROR1 antibody described herein comprises a light chain variable region having at least 80% sequence identity to SEQ ID NO: 37. In some embodiments, an anti-ROR1 antibody described herein comprises a light chain variable region having at least 85% sequence identity to SEQ ID NO: 37. In some embodiments, an anti-ROR1 antibody described herein comprises a light chain variable region having at least 90% sequence identity to SEQ ID NO: 37. In some embodiments, an anti-ROR1 antibody described herein comprises a light chain variable region having at least 95% sequence identity to SEQ ID NO: 37. In some embodiments, an anti-ROR1 antibody described herein comprises a light chain variable region having at least 99% sequence identity to SEQ ID NO: 37.

In some embodiments, an anti-ROR1 antibody described herein comprises a heavy chain variable region of SEQ ID NO: 36 and a light chain variable region of SEQ ID NO: 37.

In some embodiments, an anti-ROR1 antibody described herein comprises a heavy chain variable region comprising three complementarity determining regions and a light chain variable region comprising three CDRs, wherein the heavy chain variable region comprises a variable heavy chain (VH) CDR1 that has an amino acid sequence of SEQ ID NO: 38, a variable heavy chain (VH) CDR2 that has an amino acid sequence of SEQ ID NO: 39, a variable heavy chain (VH) CDR3 that has an amino acid sequence of SEQ ID NO: 40, and wherein the light chain variable region comprises a variable light chain (VL) CDR1 that has an amino acid sequence of SEQ ID NO: 41, a variable light (VL) CDR2 that has an amino acid sequence of SEQ ID NO: 42, and a variable light (VL) CDR3 that has an amino acid sequence of SEQ ID NO:43. In some embodiments, an anti-ROR1 antibody described herein is Y31.

In some embodiments, an anti-ROR1 antibody described herein comprises a sequence selected from Table 1.

TABLE 1
Sequence listing.

| | | SEQ ID |
|---|---|---|
| Identifier | Sequence | NO: |
| m2A2, VH | QVQLQQSGAELVRPGASVTLSCKASGYTFSDYEMHWVIQTPVHGL | 1 |
| | EWIGAIDPETGGTAYNQKFKGKAILTADKSSSTAYMELRSLTSEDSA | |
| | VYYCTGYYDYDSFTYWGQGTLVTVSA | |
| m2A2, VL | DIVMTQSQKIMSTTVGDRVSITCKASQNVDAAVAWYQQKPGQSPK | 2 |
| | LLIYSASNRYTGVPDRFTGSGSGTDFTLTISNMQSEDLADYFCQQYD | |
| | IYPYTFGGGTKLEIK | |
| VH CDR1, | DYEMH | 3 |
| m2A2, hc2A2 | | |
| and hc2A2m | | |
| VH CDR2, | AIDPETGGTAYNQKFKG | 4 |
| m2A2, hc2A2 | | |
| and hc2A2m | | |
| VH CDR3, | YYDYDSFTY | 5 |
| m2A2, hc2A2 | | |
| and hc2A2m | | |
| VL CDR1, | KASQNVDAAVA | 6 |
| m2A2, hc2A2 | | |
| and hc2A2m | | |
| VL CDR2, | SASNRYT | 7 |
| m2A2, hc2A2 | | |
| and hc2A2m | | |
| VL CDR3, | QQYDIYPYT | 8 |
| m2A2, hc2A2 | | |
| and hc2A2m | | |
| m2A2, VH | QVQLQQSGAELVRPGASVTLSCKASGYTFSDYEMHWVIQTPVHGL | 9 |
| | EWIGAIDPETGGTAYNQKFKGKAILTADKSSSTAYMELRSLTSEDSA | |
| | VYYCTGYYDYDSFTY | |
| m2A2, VL | DIVMTQSQKIMSTTVGDRVSITCKASQNVDAAVAWYQQKPGQSPK | 10 |
| | LLIYSASNRYTGVPDRFTGSGSGTDFTLTISNMQSEDLADYFCQQYD | |
| | IYPYT | |
| m2A2, Heavy | MEWSRVFIFLLSVTAGVHSQVQLQQSGAELVRPGASVTLSCKASGY | 11 |
| Chain | TFSDYEMHWVIQTPVHGLEWIGAIDPETGGTAYNQKFKGKAILTAD | |
| | KSSSTAYMELRSLTSEDSAVYYCTGYYDYDSFTYWGQGTLVTVSA | |
| | AKTTPPSVYPLAPGSAAQTNSMVTLGCLVKGYFPEPVTVTWNSGSL | |
| | SSGVHTFPAVLQSDLYTLSSSVTVPSSTWPSETVTCNVAHPASSTKV | |
| | DKKIVPRDCGCKPCICTVPEVSSVFIFPPKPKDVLTITLTPKVTCVVV | |
| | DISKDDPEVQFSWFVDDVEVHTAQTQPREEQFNSTFRSVSELPIMHQ | |
| | DWLNGKEFKCRVNSAAFPAPIEKTISKTKGRPKAPQVYTIPPPKEQM | |
| | AKDKVSLTCMITDFFPEDITVEWQWNGQPAENYKNTQPIMDTDGS | |
| | YFVYSKLNVQKSNWEAGNTFTCSVLHEGLHNHHTEKSLSHSPGK | |
| m2A2, Light | MRCLAEFLGLLVLWIPGAIGDIVMTQSQKIMSTTVGDRVSITCKASQ | 12 |
| Chain | NVDAAVAWYQQKPGQSPKLLIYSASNRYTGVPDRFTGSGSGTDFTL | |
| | TISNMQSEDLADYFCQQYDIYPYTFGGGTKLEIKRADAAPTVSIFPPS | |
| | SEQLTSGGASVVCFLNNFYPKDINVKWKIDGSERQNGVLNSWTDQ | |
| | DSKDSTYSMSSTLTLTKDEYERHNSYTCEATHKTSTSPIVKSFNRNE | |
| | C | |
| h2A2, VH | QVQLQQSGAELVRPGASVTLSCKASGYTFSDYEMHWVIQTPVHGL | 13 |
| | EWIGAIDPETGGTAYNQKFKGKAILTADKSSSTAYMELRSLTSEDSA | |
| | VYYCTGYYDYDSFTY | |
| h2A2, VL | DIVMTQSQKIMSTTVGDRVSITCKASQNVDAAVAWYQQKPGQSPK | 14 |
| | LLIYSASNRYTGVPDRFTGSGSGTDFTLTISNMQSEDLADYFCQQYD | |
| | IYPYT | |
| h2A2, Heavy | MEWSRVFIFLLSVTAGVHSQVQLQQSGAELVRPGASVTLSCKASGY | 15 |
| Chain | TFSDYEMHWVIQTPVHGLEWIGAIDPETGGTAYNQKFKGKAILTAD | |
| | KSSSTAYMELRSLTSEDSAVYYCTGYYDYDSFTYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKRVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVV | |
| | SVLTVLHQDWLNGKEYKCKVSNKALPAPIEKTISKAKGQPREPQVY | |
| | TLPPSREEMTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGK | |
| h2A2, Light | MRCLAEFLGLLVLWIPGAIGDIVMTQSQKIMSTTVGDRVSITCKASQ | 16 |
| Chain | NVDAAVAWYQQKPGQSPKLLIYSASNRYTGVPDRFTGSGSGTDFTL | |
| | TISNMQSEDLADYFCQQYDIYPYTFGGGTKLEIKRTVAAPSVFIFPPS | |
| | DEQLKSGTASVVCLLNNFYPREAKVQWKVDNALQSGNSQESVTEQ | |
| | DSKDSTYSLSSTLTLSKADYEKHKVYACEVTHQGLSSPVTKSFNRG | |
| | EC | |
| R11, VH | QSVKESEGDLVTPAGNLTLTCTASGSDINDYPISWVRQAPGKGLEW | 17 |
| | IGFINSGGSTWYASWVKGRFTISRTSTTVDLKMTSLTTDDTATYFCA | |
| | RGYSTYYCDFNIWGPGTLVTISS | |
| R11, VH | QSVKESEGDLVTPAGNLTLTCTASGSDINDYPISWVRQAPGKGLEW | 18 |
| | IGFINSGGSTWYASWVKGRFTISRTSTTVDLKMTSLTTDDTATYFCA | |
| | RGYSTYYSDFNIWGPGTLVTISS | |
| R11, VL | ELVMTQTPSSTSGAVGGTVTINCQASQSIDSNLAWFQQKPGQPPTLL | 19 |
| | IYRASNLASGVPSRFSGSRSGTEYTLTISGVQREDAATYYCLGGVGN | |
| | VSYRTSFGGGTEVVVK | |
| R11, VH CDR1 | DYPIS | 20 |
| R11, VH CDR2 | FINSGGSTWYASWVKG | 21 |
| R11, VH CDR3 | GYSTYYCDFNI | 22 |
| R11, VL CDR1 | QASQSIDSNLA | 23 |
| R11, VL CDR2 | RASNLAS | 24 |
| R11, VL CDR3 | LGGVGNVSYRTS | 25 |
| R11, Heavy | MEFGLSWVFLVALLRGVQCQSVKESEGDLVTPAGNLTLTCTASGS | 26 |
| Chain | DINDYPISWVRQAPGKGLEWIGFINSGGSTWYASWVKGRFTISRTST | |
| | TVDLKMTSLTTDDTATYFCARGYSTYYSDFNIWGPGTLVTISSASTK | |
| | GPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKR | |
| | VEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVL | |
| | TVLHQDWLNGKEYKCKVSNKALPAPIEKTISKAKGQPREPQVYTLP | |
| | PSREEMTKNQVSLTCLVKGFYPSDIAVEWESNGQPENNYKTTPPVL | |
| | DSDGSFFLYSKLTVDKSRWQQGNVFSCSVMHEALHNHYTQKSLSL | |
| | SPGK | |
| R11, Light Chain | METPAQLLFLLLLWLPDTTGELVMTQTPSSTSGAVGGTVTINCQAS | 27 |
| | QSIDSNLAWFQQKPGQPPTLLIYRASNLASGVPSRFSGSRSGTEYTLT | |
| | ISGVQREDAATYYCLGGVGNVSYRTSFGGGTEVVVKRTVAAPSVFI | |
| | FPPSDEQLKSGTASVVCLLNNFYPREAKVQWKVDNALQSGNSQES | |
| | VTEQDSKDSTYSLSSTLTLSKADYEKHKVYACEVTHQGLSSPVTKS | |
| | FNRGEC | |
| R12, VH | QEQLVESGGRLVTPGGSLTLSCKASGFDFSAYYMSWVRQAPGKGL | 28 |
| | EWIATIYPSSGKTYYATWVNGRFTISSDNAQNTVDLQMNSLTAADR | |
| | ATYFCARDSYADDGALFNIWGPGTLVTISS | |
| R12, VL | ELVLTQSPSVSAALGSPAKITCTLSSAHKTDTIDWYQQLQGEAPRYL | 29 |
| | MQVQSDGSYTKRPGVPDRFSGSSSGADRYLIIPSVQADDEADYYCG | |
| | ADYIGGYVFGGGTQLTVTG | |
| R12, VH CDR1 | AYYMS | 30 |
| R12, VH CDR2 | TIYPSSGKTYYATWVNG | 31 |
| R12, VH CDR3 | DSYADDGALFNI | 32 |
| R12, VL CDR1 | TLSSAHKTDTID | 33 |
| R12, VL CDR2 | GSYTKRP | 34 |
| R12, VL CDR3 | GADYIGGYV | 35 |
| Y31, VH | QSLEESGGRLVTPGTPLTLTCTVSGIDLNSHWMSWVRQAPGKGLE | 36 |
| | WIGIIAASGSTYYANWAKGRFTISKTSTTVDLRIASPTTEDTATYFCA | |
| | RDYGDYRLVTFNIWGPGTLVTVSS | |
| Y31, VL | ELVMTQTPSSVSAAVGGTVTINCQASQSIGSYLAWYQQKPGQPPKL | 37 |
| | LIYYASNLASGVPSRFSGSGSGTEYTLTISGVQREDAATYYCLGSLS | |
| | NSDNVFGGGTELEIL | |
| Y31, VH CDR1 | SHWMS | 38 |
| Y31, VH CDR2 | IIAASGSTYYANWAKG | 39 |
| Y31, VH CDR3 | DYGDYRLVTFNI | 40 |
| Y31, VL CDR1 | QASQSIGSYLA | 41 |
| Y31, VL CDR2 | YASNLAS | 42 |
| Y31, VL CDR3 | LGSLSNSDNV | 43 |
| human ROR1 | MHRPRRRGTRPPLLALLAALLLAARGAAAQETELSVSAELVPTSSW | 44 |
| | NISSELNKDSYLTLDEPMNNITTSLGQTAELHCKVSGNPPPTIRWFK | |
| | NDAPVVQEPRRLSFRSTIYGSRLRIRNLDTTDTGYFQCVATNGKEVV | |
| | SSTGVLFVKFGPPPTASPGYSDEYEEDGFCQPYRGIACARFIGNRTV | |
| | YMESLHMQGEIENQITAAFTMIGTSSHLSDKCSQFAIPSLCHYAFPY | |
| | CDETSSVPKPRDLCRDECEILENVLCQTEYIFARSNPMILMRLKLPN | |
| | CEDLPQPESPEAANCIRIGIPMADPINKNHKCYNSTGVDYRGTVSVT | |
| | KSGRQCQPWNSQYPHTHTFTALRFPELNGGHSYCRNPGNQKEAPW | |
| | CFTLDENFKSDLCDIPACDSKDSKEKNKMEILYILVPSVAIPLAIALL | |
| | FFFICVCRNNQKSSSAPVQRQPKHVRGQNVEMSMLNAYKPKSKAK | |
| | ELPLSAVRFMEELGECAFGKIYKGHLYLPGMDHAQLVAIKTLKDYN | |
| | NPQQWTEFQQEASLMAELHHPNIVCLLGAVTQEQPVCMLFEYINQ | |
| | GDLHEFLIMRSPHSDVGCSSDEDGTVKSSLDHGDFLHIAIQIAAGME | |
| | YLSSHFFVHKDLAARNILIGEQLHVKISDLGLSREIYSADYYRVQSK | |
| | SLLPIRWMPPEAIMYGKFSSDSDIWSFGVVLWEIFSFGLQPYYGFSN | |
| | QEVIEMVRKRQLLPCSEDCPPRMYSLMTECWNEIPSRRPRFKDIHVR | |
| | LRSWEGLSSHTSSTTPSGGNATTQTTSLSASPVSNLSNPRYPNYMFP | |
| | SQGITPQGQIAGFIGPPIPQNQRFIPINGYPIPPGYAAFPAAHYQPTGPP | |
| | RVIQHCPPPKSRSPSSASGSTSTGHVTSLPSSGSNQEANIPLLPHMSIP | |
| | NHPGGMGITVFGNKSQKPYKIDSKQASLLGDANIHGHTESMISAEL | |
| ROR1- | PTSSWNISSELNKDSYLTLDEPMNNITTSLGQTAELHCKVSGNPPPTI | 45 |
| immunoglobulin | RWFKNDAPVVQEPRRLSFRSTIYGSRLRIRNLDTTDTGYFQCVATN | |
| domain | GKEVVSSTGVLF | |
| ROR1- frizzled | EEDGFCQPYRGIACARFIGNRTVYMESLHMQGEIENQITAAFTMIGT | 46 |
| domain | SSHLSDKCSQFAIPSLCHYAFPYCDETSSVPKPRDLCRDECEILENVL | |
| | CQTEYIFARSNPMILMRLKLPNCEDLPQPESPEAANCIRI | |
| ROR1- kringle | KCYNSTGVDYRGTVSVTKSGRQCQPWNSQYPHTHTFTALRFPELN | 47 |
| domain | GGHSYCRNPGNQKEAPWCFTLDENFKSDLCDIPAC | |

Affinity refers to measures the strength of interaction between an epitope and an antibody's antigen binding site. Affinity is measured by the equilibrium dissociation constant (K_{D}). Lower values of K_{D} indicate a higher affinity, and vice versa. In some embodiments, the antibody has affinity for ROR1 of less than about 1.0×10⁻⁶M. In some embodiments, the dissociation constant is between about 1.0×10⁻⁶ and 1.0×10⁻⁷M. In other embodiments, the dissociation constant is between about 1.0×10⁻⁷ and 1.0×10⁻⁸M. In still other embodiments, the dissociation constant is between about 1.0×10⁻⁸ and 1.0×10⁻⁹M. In yet other embodiments, the dissociation constant is less than 9.9×10⁻¹⁰ M. In some embodiments, affinity is measured using art-known techniques, such as ELISA or BIACORE.

Avidity refers to measure of the overall strength of an antibody-antigen complex. In some embodiments, the antibody has avidity for ROR1 of about 10 μM or less, 5 μM or less, 2 μM or less, 1 μM or less, 500 nM or less, 400 nM or less, 300 nM or less, or 200 nM or less. In some embodiments, the antibody has avidity for ROR1 of about 100 nM or less, about 75 nM or less, about 50 nM or less, about 25 nM or less, about 10 nM or less, or about 5 nM or less. In some embodiments, the antibody has avidity for ROR1 of about 1 nM or less, about 800 pM or less, about 700 pM or less, about 600 pM or less, about 500 pM or less, about 400 pM or less, about 300 pM or less, about 200 pM or less, or about 100 pM or less. In some embodiments, avidity is measured using art-known techniques, such as ELISA or BIACORE.

### Antibody Production

In some embodiments, an anti-ROR1 antibody described herein is generated recombinantly or is synthesized chemically. In some instances, an anti-ROR1 antibody described herein is generated recombinantly, for example, either by a host cell system, or in a cell-free system.

In some instances, an anti-ROR1 antibody described herein is generated recombinantly through a host cell system. In some cases, the host cell is a eukaryotic cell (e.g., mammalian cell, insect cells, yeast cells, or plant cell) or a prokaryotic cell (e.g., gram-positive bacterium or a gram-negative bacterium).

In some embodiments, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells , 293 H cells, A549 cells, MDCK cells, CHO DG44 cells, CHO-S cells, CHO-K1 cells, Expi293™ cells, Flp-In™T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cell include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris yeast strains such as GS115, KM71H, SMD1168, SMD1168H, and X-33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™ DH12S™, INV110, TOP10F′, INVαF, TOP10/P3, ccdB Survival, PIR1, PIR2, Stb12™, Stb13™, or Stb14™.

In some instances, suitable polynucleic acid molecules or vectors for the production of an anti-ROR1 antibody described herein include any suitable vectors derived from either a eukaryotic or prokaryotic sources. Exemplary polynucleic acid molecules or vectors include vectors from bacteria (e.g., E. coli), insects, yeast (e.g., Pichia pastoris), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBacl, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is used for the production of an anti-ROR1 antibody described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, or HeLa cells. Exemplary cell-free systems include E. coli S30 Extract system, E. coli T7 S30 system, or PURExpress®.

### Payload

Disclosed herein, in some embodiments, are compositions comprising an antibody-payload conjugate. In some instances, the payload conjugate is a cytotoxic payload. In some cases, the payload comprises a microtubule disrupting agent, a DNA modifying agent or a combination thereof.

### Microtubule Disrupting Agent

In some embodiments, the payload comprises a microtubule disrupting agent. Exemplary microtubule disrupting agents include, but are not limited to, 2-methoxyestradiol, chalcones, colchicine, combretastatin, dictyostatin, discodermolide, eleutherobin, epothilone, laulimalide, peloruside A, podophyllotoxin, taxane, cryptophycin, halichondrin B, maytansine, phomopsin A, rhizoxin, spongistatin, tubulysin, vinca alkaloid, noscapinoid, auristatin, dolastain, or derivatives or analogs thereof. In some embodiments, the payload is combretastatin or a derivative or analog thereof. In some embodiments, an analog of combretastatin is ombrabulin. In some embodiments, the epothilone is epothilone B, patupilone, ixabepilone, sagopilone, BMS-310705, or BMS-247550. In some embodiments, the tubulysin is a tubulysin analog or derivative such as described in U.S. Pat. Nos. 8,580,820 and 8,980,833 and in U.S. Publication Nos. 20130217638, 20130224228, and 201400363454. In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansinoid derivative or analog such as described in U.S. Pat. Nos. 5,208,020, 5,416,064, 7,276,497, and 6,716,821 or U.S. Publication Nos. 2013029900 and US20130323268. In some embodiments, the taxane is paclitaxel or docetaxel. In some embodiments, the vica alkaloid is vinblastine, vincristine, vindesine, vinorelbine, desoxyvincaminol, vincaminol, vincamajine, vineridine, vinburnine, vinpocetine, or vincamine.

### Dolastatin and Auristatin

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714 and 8,871,720. In some embodiments, the dolastatin 15 analog is cemadotin or tasidotin.

### DNA Modifying Agent

In some embodiments, the payload comprises a DNA modifying agent. In some embodiments, the DNA modifying agent comprises amsacrine, anthracycline, camptothecin, doxorubicin, duocarmycin, enediyne, etoposide, indolinobenzodiazepine, netropsin, teniposide, pyrrolobenzodiazepine, or derivatives or analogs thereof. In some embodiments, the anthracycline is doxorubicin, daunorubicin, epirubicin, idarubicin, mitomycin-C, dactinomycin, mithramycin, nemorubicin, pixantrone, sabarubicin, or valrubicin. In some embodiments, the analog of camptothecin is topotecan, irinotecan, silatecan, cositecan, exatecan, lurtotecan, gimatecan, belotecan, rubitecan, or SN-38. In some embodiments, the duocarmycin is duocarmycin A, duocarmycin B 1, duocarmycin B2, duocarmycin C1, duocarmycin C2, duocarmycin D, duocarmycin SA, or CC-1065. In some embodiments, the enediyne is a calicheamicin, esperamicin, or dynemicin A.

### Pyrrolobenzodiazepine

Pyrrolobenzodiazepine (PBDs) are a class of sequence-selective DNA minor-groove binding crosslinking agents. PBD dimers are particularly potent because of their cell cycle-independent activity and because their integration minimally distorts DNA, increasing the likelihood of evasion of DNA damage repair responses.

In some embodiments, the payload is pyrrolobenzodiazepine. In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120 (Table 2). In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

TABLE 2
Symmetric pyyrolobenzodiazepine dimers.

| PBD | |
|---|---|
| dimer | Structure |
| SJG-136 (SG2000) | 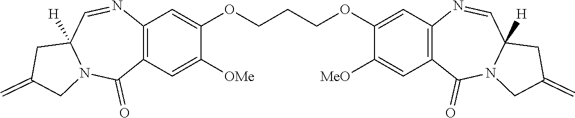 |
| ZC-423 (SG2285) | 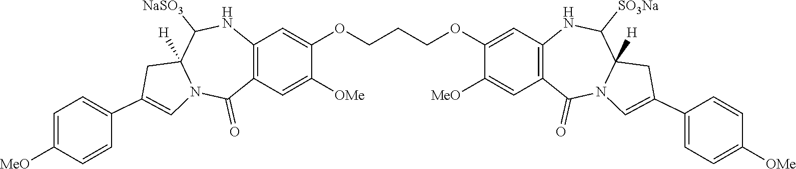 |
| SJG-720 | 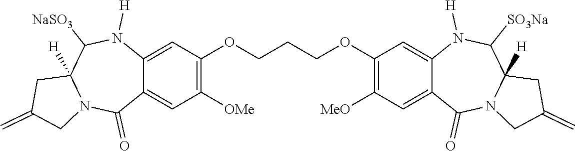 |
| SJG-738 | 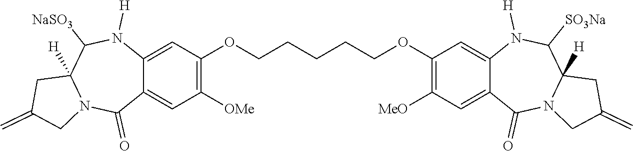 |
| ZC-207 (SG2202) | 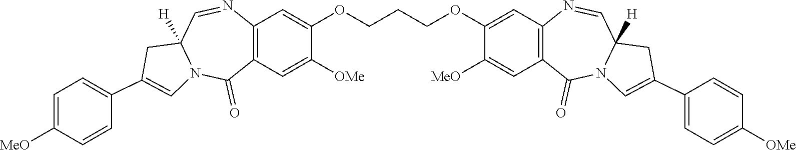 [SMILES: COc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1] |
| DSB-120 | 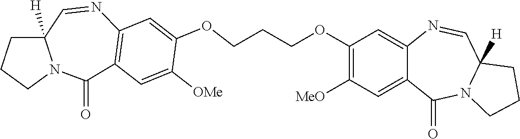 [SMILES: COc1cc2c(cc1OCCCOc1cc3c(cc1OC)C(=O)N1CCC[C@H]1C=N3)N=C[C@@H]1CCCN1C2=O] |

In some embodiments, one or more payload is conjugated to an antibody described herein. In some instances, about 1, 2, 3, 4, 5, 6, 7, 8, 9, 10, 11, 12, 13, 14, 15, 16, or more payloads are conjugated to an antibody described herein. In some cases, about 1 payload is conjugated to an antibody described herein. In some cases, about 2 payloads are conjugated to an antibody described herein. In some cases, about 3 payloads are conjugated to an antibody described herein. In some cases, about 4 payloads are conjugated to an antibody described herein. In some cases, about 5 payloads are conjugated to an antibody described herein. In some cases, about 6 payloads are conjugated to an antibody described herein. In some cases, about 7 payloads are conjugated to an antibody described herein. In some cases, about 8 payloads are conjugated to an antibody described herein. In some cases, about 9 payloads are conjugated to an antibody described herein. In some cases, about 10 payloads are conjugated to an antibody described herein. In some cases, about 11 payloads are conjugated to an antibody described herein. In some cases, about 12 payloads are conjugated to an antibody described herein. In some cases, about 13 payloads are conjugated to an antibody described herein. In some cases, about 14 payloads are conjugated to an antibody described herein. In some cases, about 15 payloads are conjugated to an antibody described herein. In some cases, about 16 payloads are conjugated to an antibody described herein.

In some embodiments, the number of payloads conjugated to an antibody described herein forms a ratio. In some cases, the ratio is referred to as a DAR (drug-to-antibody) ratio. In some instances, the DAR ratio of payload to an antibody described herein is about 1, 2, 3, 4, 5, 6, 7, 8, 9, 10, 11, 12, 13, 14, 15, 16 or greater. In some instances, the DAR ratio of payload to an antibody described herein is about 1 or greater. In some instances, the DAR ratio of payload to an antibody described herein is about 2 or greater. In some instances, the DAR ratio of payload to an antibody described herein is about 4 or greater. In some instances, the DAR ratio of payload to an antibody described herein is about 6 or greater. In some instances, the DAR ratio of payload to an antibody described herein is about 8 or greater. In some instances, the DAR ratio of payload to an antibody described herein is about 12 or greater.

In some instances, the DAR ratio of payload to an antibody described herein is 1, 2, 3, 4, 5, 6, 7, 8, 9, 10, 11, 12, 13, 14, 15 or 16. In some instances, the DAR ratio of payload to an antibody described herein is 1. In some instances, the DAR ratio of payload to an antibody described herein is 2. In some instances, the DAR ratio of payload to an antibody described herein is 4. In some instances, the DAR ratio of payload to an antibody described herein is 6. In some instances, the DAR ratio of payload to an antibody described herein is 8. In some instances, the DAR ratio of payload to an antibody described herein is 12.

### Linkers

A linker provides a functional handle for efficient conjugation of a payload to an antibody. More sophisticated linkers increase effector solubility, improve stability throughout the production process, prevent premature drug release, and facilitate the liberation of active drug at the target. In some instances, different aspects of linker chemistry include the functionality that allows conjugation to antibody, the mechanism for drug release, and the physical properties of the linker itself. Non-cleavable linkers require antibody degradation within the lysosome following antibody-payload conjugate internalization to release the active drug. Cleavable linkers respond to physiological stimuli, such as low pH, high glutathione concentration, and proteolytic cleavage.

In some embodiments, the anti-ROR1 antibody-payload conjugate further comprises a linker moiety. In some embodiments, the linker comprises a homobifunctional linker or a heterobifunctional linker. In some embodiments, the linker is a cleavable linker or a non-cleavable linker.

In some instances, the linker comprises a homobifuctional linker. Exemplary homobifuctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (ρNPDP), ρ-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker is a cleavable linker. In some embodiments, the cleavable linker is a dipeptide linker. In some embodiments, the dipeptide linker is valine-citrulline (Val-Cit), phenylalanine-lysine (Phe-Lys), valine-alanine (Val-Ala) and valine-lysine (Val-Lys). In some embodiments, the dipeptide linker is valine-citrulline.

In some embodiments, the linker comprises a self-immolative linker moiety. In some embodiments, the self-immolative linker moiety comprises p-aminobenzyl alcohol (PAB), p-aminobenzyoxycarbonyl (PABC), or derivatives or analogs thereof. In some embodiments, the linker comprises a dipeptide linker moiety and a self-immolative linker moiety. In some embodiments, the self-immolative linker moiety is such as described in U.S. Pat. No. 9,089,614 and PCT Publication No. WO2015038426.

In some embodiments, the cleavable linker is glucuronide. In some embodiments, the cleavable linker is an acid-cleavable linker. In some embodiments, the acid-cleavable linker is hydrazine. In some embodiments, the cleavable linker is a reducible linker.

In some embodiments, the linker comprises a maleimide group. In some instances, the maleimide group is also referred to as a maleimide spacer. In some instances, the maleimide group further comprises a caproic acid, forming maleimidocaproyl (mc). In some cases, the linker comprises maleimidocaproyl (mc). In some cases, linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylat (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of thiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some instances, a linker comprises a polyalkylene oxide (e.g., polyethylene glycol) compound. In some embodiments, a polyalkylene oxide (e.g., PEG) is a polydisperse or monodisperse compound. In some instances, a polydispersed PEG comprises disperse distribution of different molecular weight of the material, characterized by mean weight (weight average) size and dispersity. In some instances, a monodisperse PEG comprises one size of PEG molecules.

In some embodiments, the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG (dPEG). Discrete PEG comprises a linear chain of four to 48 ethylene oxide units with a single molecular weight. Branched discrete PEG comprises from three to nine linear chains of discrete PEG. In some cases, the discrete PEG is dPEG® from Quanta Biodesign Ltd.

Covalent attachment of polyethylene glycol (PEG) to proteins has been shown to reduce the antigenicity of the compounds, decrease toxicity, prolong half-life, and increase solubility. In some embodiments, the linker comprises a PEG moiety. In some embodiments, the PEG moiety is a branched or multi-arm PEG moiety. In some embodiments, a plurality of payloads is attached to the branched or multi-arm PEG moiety. In some embodiments, the PEG moiety comprises N-hydroxy-succinimide (NHS) esters at both ends of the PEG moiety. In some embodiments, the PEG moiety comprises a maleimide group at both ends of the PEG moiety. In some embodiments, the PEG moiety comprises an N-hydroxy-succinimide (NHS) esters at one end of the PEG moiety and a maleimide group at the other end of the PEG moiety. In some embodiments, the PEG moiety is a PEG moiety such as described in PCT Publication WO2015057699.

In some embodiments, the linker comprises a self-stabilizing linker, such as described in U.S. Publication 20130309256. In some embodiments, the linker comprises a hydrophilic linker, such as describe in PCT Publication WO2014080251. In some embodiments, the linker is a lysosome-cleavable linker, such as described in U.S. Publication 20150037360. In some embodiments, the linker comprises a linker with improved stability, such as describe in PCT Publication WO2014197854. In some embodiments, the linker is a sulfonamide based linker, such as described in PCT Publication WO2015095953 and U.S. Publication 20140315954. In some embodiments, the linker is a linker such as described in PCT Publication WO2014145090 and 2014177042 and U.S. Publication 20140294851.

### Conjugation Chemistry

The conjugation method of payload to antibody determines drug load stoichiometry (the DAR), species homogeneity, antibody structural stability, and binding capacity.

Reactive side chains of naturally occurring amino acids, such as lysine and cysteine, are attractive sites of conjugation. The main advantage of linkage through native residues is facile reactivity that does not require preliminary processing/modification of the antibody. The main disadvantages of these methods are the variability and heterogeneity of the resulting products, as nonselective ligation results in a large number of isoforms permutations possible.

The majority of antibody-payload conjugates are built on IgG1 scaffolds. The IgG scaffold has over 80 lysines, with over 20 of these residues found at highly solvent-accessible sites leading to a wide range of possible DARs. Cysteines are less prevalent than lysines in IgGs, and due to the limited number of potential sites, this method produces antibody-payload conjugates that are easier to characterize than the lysine coupling method, a feature that has been correlated with increased efficacy. In some embodiments, conjugation of a payload to the antibody occurs on a cysteine residue. In some embodiments, conjugation of a payload to the antibody occurs on a lysine residue. In some embodiments, dual conjugation of two payloads occurs. In some embodiments, the dual conjugation comprises a lysine residue and a cysteine residue. An example of dual conjugation (K-lock+C-lock) of two different payloads to a cysteine residue (C-lock) and a lysine (K-Lock) residue is described in U.S. Publications 20150105539 and 20150105540.

In some embodiments, a payload is conjugated to a modified native antibody or a site-specific engineered antibody. Conjugation of a payload to a modified native antibody or a site-specific engineered antibody allows more homogenous conjugates to be produced and allows for

### Modification of Native Antibodies

### Reducing the Number of Interchain Disulfide Bonds

Antibodies comprise inter- and intra-chain disulfide bonds. Reduction of the interchain disulfide bonds yields cysteine residues to which a payload can be conjugated. In some embodiments, a payload is conjugated to a reduced interchain disulfide bond of the antibody. In some embodiments, a disulfide reducing agent is used to reduce the interchain disulfide bond. In some embodiments, the disulfide reducing agent is: dithiothreitol (DTT), tris(2-carboxyethyl)phosphine (TCEP), or 2-mercaptoethanol.

### Selective Glycan Targeting

Antibodies are glycosylated at conserved positions in their constant region. Most antibodies possess an N-glycosylation site at the conserved N297 residue of the Fc region. In some embodiments, the payload is attached to a glycosylated N297. In some embodiments, the antibody is glycosylated at a different amino acid from N297. In some embodiments, the payload is attached to the glycosylated amino acid.

### Engineered Antibodies

### Engineered Cysteine

In some embodiments, it is desirable to create cysteine engineered antibodies, e.g., “thioMAbs,” in which one or more residues of an antibody are substituted with cysteine residues. In some embodiments, the substituted residues occur at accessible sites of the antibody. In some embodiments, reactive thiol groups are positioned at sites for conjugation to other moieties, such as drug moieties or linker-drug moieties, to create an immunoconjugate. In some embodiments, any one or more of the following residues are substituted with cysteine: V205 (Kabat numbering) of the light chain; A118 (EU numbering) of the heavy chain; and 5400 (EU numbering) of the heavy chain Fc region. Cysteine engineered antibodies may be generated as described (See, e.g., U.S. Pat. No. 7,521,541). In some embodiments one, two, three, four, five, six, seven, eight or more residues of an antibody are substituted with a cysteine residue. In some embodiments, the engineered cysteines are introduced on the heavy chain, light chain, or Fc.

### Unnatural Amino Acids

In some embodiments, the antibody comprises an unnatural amino acid. In some embodiments, the unnatural amino acid comprises a bioorthogonal functional group. In some embodiments, the biorthogonal functional group is incorporated into the antibody using a tRNA/aminoacyl-tRNA synthetase pair. In some embodiments the unnatural amino acid is p-acetylphenylalanine (pAcF, pAcPhe) or p-azidomethylphenylalanine (pAzF). In some embodiments, the unnatural amino acid is conjugated via oxime ligation. In some embodiments, the unnatural amino acid is conjugated by copper-free click chemistry.

In some embodiments, the antibody comprises a selenocysteine. In some embodiments, the antibody comprises a modified selenocysteine.

### Enzymatic Conjugation

Another approach to achieving site-selective modification is using enzymes that react with a particular amino acid in a specific amino acid sequence.

Transglutaminases (TG) catalyse the formation of amide bonds between the primary amine of a lysine and the amide group of a glutamine. Bacterial TG isolated from Streptoverticillium mobaraense has an atypical catalytic site and does not catalyse a reaction with any of the naturally occurring glutamine resides. Rather, this TG will recognize a glutamine (Q) tag. In some embodiment, the antibody comprises a Q tag. In some embodiments, the Q tag is LLQG (SEQ ID NO: 48). In some embodiments, the antibody is conjugated to the payload at the Q tag.

### SMARTag™ Technology

The ability to take the aldehyde tag out of its natural context and place it into any desired protein with continued Cys to FGly conversion is the key element to SMARTag technology SMARTag™ technology uses a unique chemoenzymatic method using the naturally occurring endogenous formylglycine-generating enzyme (FGE). FGE cotranslationally converts the cysteine in the pentapeptide sequence CXPXR, where X represents any neutral amino acid residue, to a formylglycine (fGly) residue. During protein production FGE oxidizes the cysteine in the consensus sequence to formylglycine. This co-translational modification removes the need to generate and purify a second recombinant enzyme in addition to the protein of interest. The cysteine in the aldehyde tag pentapeptide is converted with exquisite fidelity and allows the aldehyde tag to be placed at a variety of sites on the protein and retain high conversion to fGly (>95%) with exceptional protein titers (5 g/L) across a variety of tag placements.

In some embodiments, the antibody comprises a CXPXR sequence. In some embodiments, the X of the CXPXR sequence is any amino acid. In some embodiments, the X of the CXPXR sequence is serine, threonine, alanine, or glycine. In some embodiments, the antibody is conjugated to the payload at the aldehyde converted from the cysteine of the CXPXR sequence. In some embodiments, the antibody-drug conjugate is conjugated using SMARTag™ technology, such as described in U.S. Publication No. 20100210543.

### Method of Treatment

Disclosed herein, in certain embodiments, are methods of treating a subject having cancer comprising administering to a subject a therapeutically effective amount of an anti-ROR1 antibody-payload conjugate. In some instances, a method described herein comprises administering to a subject a therapeutically effective amount of a pharmaceutical composition comprising an anti-ROR1 antibody-payload conjugate, in which the subject has adrenal cancer, B-cell lymphoma, bladder cancer, breast cancer, colorectal cancer, liver cancer, lung cancer, pancreatic cancer, prostate cancer, renal cell carcinoma, skin cancer, stomach cancer, or acute lymphoblastic leukemia. In some embodiments, the cancer is a metastatic cancer. In some cases, the cancer is a relapsed or a refractory cancer.

In some embodiments, a therapeutically effective amount of a pharmaceutical composition comprising an anti-ROR1 antibody-payload conjugate is administered to a subject having B-cell lymphoma. In some embodiments, B-cell lymphoma comprises Hodgkin's lymphoma and non-Hodgkin's lymphoma. In some embodiments, non-Hodgkin's lymphoma comprises diffuse large B-cell lymphoma (DLBCL), follicular lymphoma, marginal zone B-cell lymphoma (MZL), chronic lymphocytic leukemia (CLL), small lymphocytic lymphoma (SLL), Mantle cell lymphoma (MCL), Burkitt's lymphoma, Waldenstrom's macroglobulinemia, nodal marginal zone B cell lymphoma (NMZL), splenic marginal zone lymphoma (SMZL), intravascular large B-cell lymphoma, primary effusion lymphoma, lymphomatoid granulomatosis, primary central nervous system lymphoma and plasmablastic lymphoma.

In some embodiments, a therapeutically effective amount of a pharmaceutical composition comprising an anti-ROR1 antibody-payload conjugate described herein is administered to a subject having bladder cancer. In some embodiments, bladder cancer comprises luminal bladder cancer and basal bladder cancer.

In some embodiments, a therapeutically effective amount of a pharmaceutical composition comprising an anti-ROR1 antibody-payload conjugate described herein is administered to a subject having breast cancer. In some embodiments, breast cancer comprises cancer cells positive for estrogen receptor (ER), progesterone receptor (PR), overexpression of HER2/neu, or a combination thereof. In some embodiments, breast cancer comprises cancer cells which are triple negative (i.e. ER-, PR-, and HER2-).

In some embodiments, a therapeutically effective amount of a pharmaceutical composition comprising an anti-ROR1 antibody-payload conjugate described herein is administered to a subject having colorectal cancer. In some embodiments, colorectal cancer comprises adenocarcinoma, lymphoma, and squamous cell carcinoma.

In some embodiments, a therapeutically effective amount of a pharmaceutical composition comprising an anti-ROR1 antibody-payload conjugate described herein is administered to a subject having liver cancer. In some embodiments, liver cancer comprises hepatocellular carcinoma (HCC), cholangiocarcinoma, angiosarcoma, or heptoblastoma.

In some embodiments, a therapeutically effective amount of a pharmaceutical composition comprising an anti-ROR1 antibody-payload conjugate described herein is administered to a subject having lung cancer. In some embodiments, lung cancer comprises non-small cell lung cancer (NSCLC). In some embodiments the NSCLC comprises adenocarcinoma, squamous-cell carcinoma, or large-cell carcinoma. In some embodiments, lung cancer comprises small cell lung cancer (SCLC).

In some embodiments, a therapeutically effective amount of a pharmaceutical composition comprising an anti-ROR1 antibody-payload conjugate described herein is administered to a subject having pancreatic cancer. In some embodiments, pancreatic cancer comprises an exocrine tumor or an endocrine tumor. In some embodiments, the exocrine tumor is an adenocarcinoma, acinar cell carcinoma, intraductal papillary-mucinous neoplasm (IPMN), or mucinous cystadenocarcinoma. In some embodiments, the endocrine tumor is a gastrinoma, glucagonoma, insulinoma, somatostatinoma, VIPoma, nonfunctional islet cell tumor, or multiple endocrine neoplasia type-1 (MEN1).

In some embodiments, a therapeutically effective amount of a pharmaceutical composition comprising an anti-ROR1 antibody-payload conjugate described herein is administered to a subject having renal cell carcinoma (RCC) cancer. In some embodiments, renal cell carcinoma comprises clear cell RCC, papillary RCC, chromophobe RCC, collecting duct RCC, multilocular cystic RCC, medullary carcinoma, mucinous tubular and spindle cell carcinoma, and neuroblastoma-associated RCC.

In some embodiments, a therapeutically effective amount of a pharmaceutical composition comprising an anti-ROR1 antibody-payload conjugate described herein is administering to the subject having stomach (gastric) cancer. In some embodiments, stomach cancer comprises a gastric adenocarcinoma. In some embodiments, the gastric adenocarcinoma comprises intestinal type adenocarcinoma and diffuse type adenocarcinoma. In some embodiments, stomach cancer comprises a lymphoma, a carcinoid, or a stromal tumor. In some embodiments, the lymphoma is a MALT lymphoma or a diffuse large B-cell lymphoma of the stomach.

In some embodiments, a therapeutically effective amount of a pharmaceutical composition comprising an anti-ROR1 antibody-payload conjugate described herein is administered to a subject having acute lymphoblastic leukemia (ALL). In some embodiments, acute lymphoblastic leukemia comprises precursor B-cell ALL, precursor T-cell ALL, Burkitt-type ALL, and Philadelphia chromosome positive ALL.

In some embodiments, a therapeutically effective amount of a pharmaceutical composition comprising an anti-ROR1 antibody-payload conjugate described herein is administered to a subject having adrenal cancer.

In some embodiments, a therapeutically effective amount of a pharmaceutical composition comprising an anti-ROR1 antibody-payload conjugate described herein is administered to a subject having skin cancer. In some instances, a skin cancer comprises basal cell cancer, melanoma or squamous cell skin carcinoma.

In some embodiments, a therapeutically effective amount of a pharmaceutical composition comprising an anti-ROR1 antibody-payload conjugate described herein is administered to a subject having prostate cancer. In some embodiments, prostate cancer comprises acinar adenocarcinoma, ductal adenocarcinoma, transitional cell (or urothelial) cancer, squamous cell cancer, carcinoid, small cell cancer, or sarcomas and sarcomatoid cancers.

### Combination Therapy

In some embodiments, a method described herein further comprises administering an additional therapeutic agent. In some embodiments, the additional therapeutic agent comprises an antimetabolite, an intercalating agent, a platinum derivative, alkylating agent, an antimitotic agent, a topoisomerase inhibitor, a cell cycle inhibitor, a microtubule agent, a proteasome inhibitor, an antibody, chemotherapy agent or a combination thereof. In some embodiments, the additional therapeutic agent is an antimetabolite. In some embodiments, the antimetabolite is an antifolate, fluoropyrimidine, cytosine arabinoside, or an analogue of purine or adenosine. In some embodiments, the antifolate is methotrexate, pemetrexed, proguanil, pyrimethamine, or trimethoprim. In some embodiments, the additional therapeutic agent is an intercalating agent. In some embodiments, the intercalating agent is an anthracycline. In some embodiments, the anthracycline is doxorubicin, daunomycin, epirubicin, idarubicin, mitomycin-C, dactinomycin, or mithramycin. In some embodiments, the additional therapeutic agent is a platinum derivative. In some embodiments, the platinum derivative is cisplatin or carboplatin. In some embodiments, the additional therapeutic agent is an alkylating agent. In some embodiments, the alkylating agent is nitrogen mustard, melphalan, chlorambucil, busulphan, cyclophosphamide, ifosfamide, nitrosoureas, or thiotepa. In some embodiments, the additional therapeutic agent is an antimitotic agent. In some embodiments, the antimitotic agent is a vinca alkaloid or a taxane. In some embodiments, the vinca alkaloid is vincristine. In some embodiments, the taxane is paclitaxel or docetaxel. In some embodiments, the additional therapeutic agent is a topoisomerase inhibitor. In some embodiments, the topoisomerase inhibitor is etoposide, teniposide, amsacrine, or topotecan. In some embodiments, the additional therapeutic agent is a cell cycle inhibitor. In some embodiments, the cell cycle inhibitor is flavopyridol. In some embodiments, the additional therapeutic agent is a microtubule agent. In some embodiments, the microtubule agent is an epothilone, discodermolide analog, or eleutherobin analog. In some embodiments, the additional therapeutic agent is a proteasome inhibitor. In some embodiments, the proteasome inhibitor is bortezomib, carfilzomib, or ixazomib. In some embodiments, the additional therapeutic agent is an antibody. In some embodiments, the antibody is rituximab or alemtuzumab. In some embodiments, the additional therapeutic agent is prednisone. In some embodiments, the additional therapeutic agent is OSU-2S (Mani et al. Exp Hematol. 2015 September; 43(9):770-4). In some embodiments, the additional therapeutic agent is a chemotherapeutic agent, a radiotherapeutic agent, or a combination thereof.

### Pharmaceutical Composition and Formulation

Pharmaceutical compositions and formulations described herein comprise an anti-ROR1 antibody-payload conjugate described herein and a pharmaceutically acceptable carrier. In some embodiments, the pharmaceutically acceptable carrier is any suitable pharmaceutically acceptable carrier. The term “pharmaceutically acceptable carrier” as used herein means one or more compatible solid or liquid fillers, diluents, other excipients, or encapsulating substances which are suitable for administration into a human or veterinary patient (e.g., a physiologically acceptable carrier or a pharmacologically acceptable carrier). The term “carrier” denotes an organic or inorganic ingredient, natural or synthetic, with which the antibody-payload conjugate is combined to facilitate the application. In some embodiments, the pharmaceutically acceptable carrier is co-mingled with one or more of the antibody-payload conjugates and with each other, when more than one pharmaceutically acceptable carrier is present in the composition in a manner so as not to substantially impair the desired pharmaceutical efficacy.

“Pharmaceutically acceptable” materials typically are capable of administration to a subject without the production of significant undesirable physiological effects such as nausea, dizziness, rash, or gastric upset. It is, for example, desirable for a composition comprising a pharmaceutically acceptable carrier not to be immunogenic when administered to a human patient for therapeutic purposes.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric, and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases, and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite, and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcoce®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumarate, a hydrocarbon such as mineral oil, or hydrogenated vegetable oil such as hydrogenated soybean oil) (Sterotex®, higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowa™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethyl cellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

In some embodiments, a pharmaceutical compositions described herein is formulated for administration to a subject via any conventional means including, but not limited to, oral, parenteral, buccal, intranasal, rectal or transdermal administration routes. In some instances, a pharmaceutical composition described herein is formulated for parenteral administration route. In some cases, parenteral administration route comprises intravenous, subcutaneous, intramuscular, intra-arterial, intraosseous infusion, intracerebral, intracerebroventricular, or intrathecal administration route. In some instances, a pharmaceutical composition described herein is formulated for intravenous, subcutaneous, intramuscular, intra-arterial, intraosseous infusion, intracerebral, intracerebroventricular, or intrathecal administration route.

In some instances, a pharmaceutical composition described herein is presented in any unit dosage form and is prepared by any suitable method, many of which are well known in the art of pharmacy. Such methods include the step of bringing the antibody-payload conjugate into association with a carrier that constitutes one or more accessory ingredients. Exemplary suitable dosage forms include, but are not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the composition is prepared by uniformly and intimately bringing the antibody-payload conjugate into association with a liquid carrier, a finely divided solid carrier, or both, and then, if necessary, shaping the product.

In some embodiments, a composition suitable for parenteral administration comprises a sterile aqueous preparation of the inventive composition, which preferably is isotonic with the blood of the recipient. In some embodiments, the aqueous preparation is formulated according to known methods using suitable dispersing or wetting agents and suspending agents. In some embodiments the sterile injectable preparation is a sterile injectable solution or suspension in a non-toxic parenterally-acceptable diluent or solvent, for example, as a solution in 1,3-butane diol. In some embodiments, the vehicle or solvent is water, Ringer's solution, or isotonic sodium chloride solution. In some embodiments, sterile, fixed oils are conventionally employed as a solvent or suspending medium. In some embodiments, the oil is a synthetic mono-or di-glycerides. In some embodiments a fatty acids such as oleic acid is used in the preparation of injectables. In some embodiments, carrier formulations suitable for oral, subcutaneous, intravenous, intramuscular, etc. administrations are found in Remington's Pharmaceutical Sciences, Mack Publishing Co., Easton, Pa.

In some embodiments, delivery systems useful in the context of the composition include time-released, delayed release, and sustained release delivery systems such that the delivery of the composition occurs prior to, and with sufficient time to cause, sensitization of the site to be treated.

In some embodiments, any suitable release delivery system is used. In some embodiments, suitable release delivery system include polymer base systems such as poly(lactide-glycolide), copolyoxalates, polycaprolactones, polyesteramides, polyorthoesters, polyhydroxybutyric acid, and polyanhydrides. In some embodiments, microcapsules of the foregoing polymers containing drugs are described in, for example, U.S. Pat. No. 5,075,109. In some embodiments, delivery systems also include non-polymer systems that are lipids including sterols such as cholesterol, cholesterol esters, and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In some embodiments, a pump-based hardware delivery system is used. In some embodiments, the pump-based hardware delivery system is adapted for implantation.

### Therapeutic Regimens

In some embodiments, one or more pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and EDS° . Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein are kits and articles of manufacture suitable for carrying out the methods disclosed herein. In some embodiments, the kit comprises two or more components required for performing a therapeutic method described herein. In some embodiments, kit components include, but are not limited to, one or more antibody-payload conjugates of the invention, appropriate reagents, and/or equipment. In some embodiments, the kit is packaged in a vial, pouch, ampoule, and/or any container suitable for a therapeutic method. Additional examples of pharmaceutical packaging materials include, but are not limited to, blister packs, bottles, tubes, bags, containers, bottles, and any packaging material suitable for a selected formulation and optionally intended mode of administration and treatment. In some embodiments, kit components are provided as concentrates (including lyophilized compositions), which are further diluted prior to use or provided at the concentration of use. In some embodiments, when the antibody-payload conjugate is for use in vivo, a single dosage is provided in a sterilized container having the desired amount and concentration of antibody-payload conjugate.

In some cases, a kit includes labels listing contents and/or instructions for use, and package inserts with instructions for use. A set of instructions will also typically be included.

In some embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### EXAMPLES

### Example 1: Bioinformatic Characterization of ROR1 Over-Expression

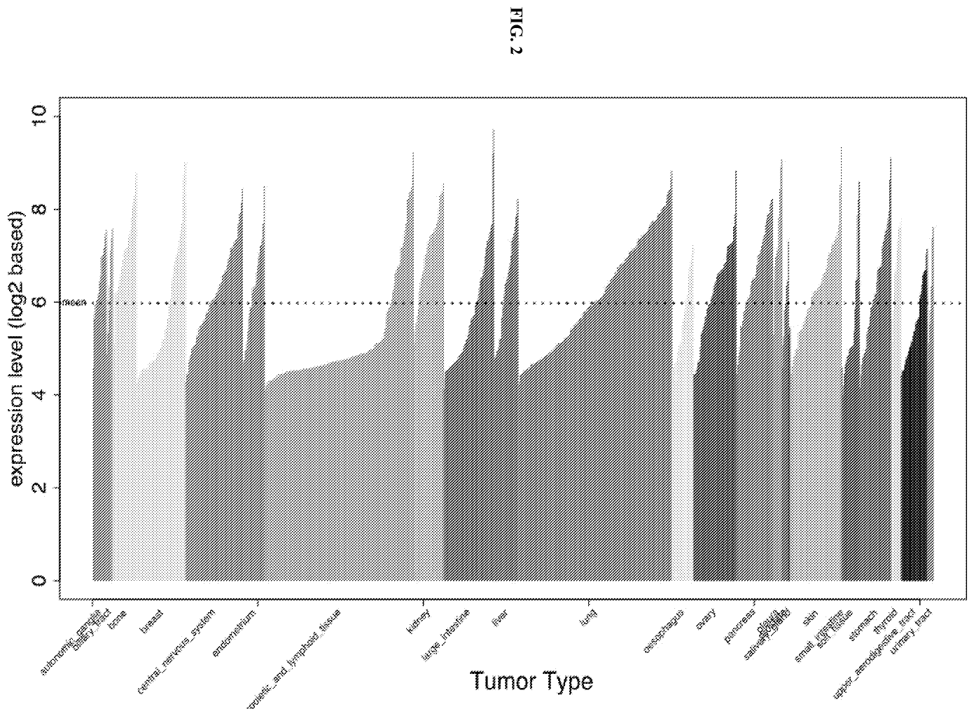

mRNA expression data for ROR1 was obtained for 650 cancer cell lines from the CrownBio Database (FIG. 2). Cells lines positive and negative for ROR1 expression were identified (Table 3).

TABLE 3
Examples of ROR1(+) and ROR1(−) cell lines.

| ROR1(+) cell line | ROR1(−) cell line |
|---|---|
| Jeko-1 (B-cell lymphoma) | Ramos (B-cell lymphoma) |
| HT29 (colon) | HCT116 (colon) |
| T24 (bladder) | RT24 (bladder) |
| Kasumi-2 (ALL) | NALM-6 (ALL) |
| MD-MB231 (breast) | T47D (breast) |
| Panc-1 (pancreatic) | MiaPaCa-2 (pancreatic) |
| A549 (lung) | MOLT-16 (T cell leukemia) |
| 789-0 (renal) | |

### Example 2: Quantitative Flow Cytometry of ROR1 Binding Sites

Quantitative flow cytometry of ROR1 binding sites per cell was assessed using 2A2 anti-ROR1 antibodies (Table 4).

TABLE 4
R0R1 binding sites per cell.

| | MFI (Geom. mean of FL2) | | | Ab binding capacity/cell | | | Specific m2A2 |
|---|---|---|---|---|---|---|---|
| | Cell | | | Cell | | | binding |
| Cell line | only | mIgG | m2A2 | only | mIgG | m2A2 | capacity/cell |
| Daudi | 328.77 | 327.08 | 341.85 | 229 | 227 | 238 | 11 |
| Jeko-1 | 340.33 | 338.17 | 9488.05 | 237 | 235 | 6994 | 6759 |
| Namalwa | 319.84 | 294.32 | 11591.17 | 222 | 204 | 8574 | 8370 |
| MDA-MD-231 | 942.92 | 931.42 | 11408.09 | 668 | 660 | 8436 | 7776 |
| NCI-H226 | 1536.32 | 1396.62 | 8832.99 | 1097 | 996 | 6503 | 5507 |
| NCI-H2228 | 894.37 | 849.21 | 12071.82 | 633 | 600 | 8936 | 8336 |
| MHCC97H | 1099.77 | 1066.18 | 6460.19 | 781 | 757 | 4731 | 3974 |
| HCCLM3 | 1420.81 | 1434.6 | 7892.06 | 1014 | 1024 | 5799 | 4775 |
| KATO-III | 1075.85 | 1070.02 | 5629.06 | 764 | 760 | 4112 | 3352 |
| MKN-45 | 641.4 | 659.22 | 1616.83 | 451 | 464 | 1156 | 692 |

### Example 3: Production and Characterization of Anti-ROR1 Antibodies

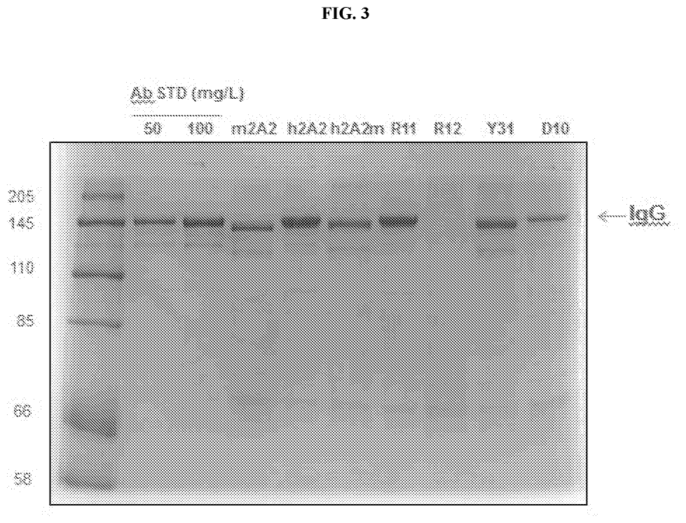

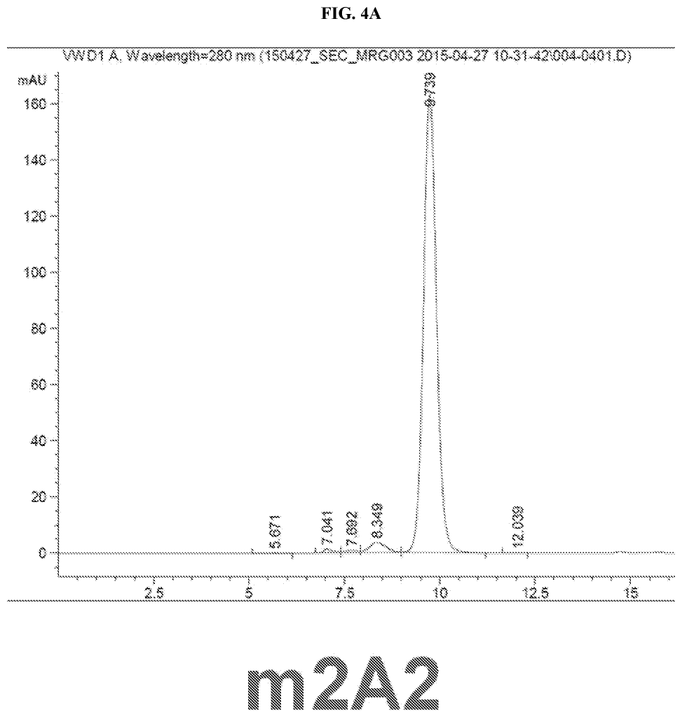

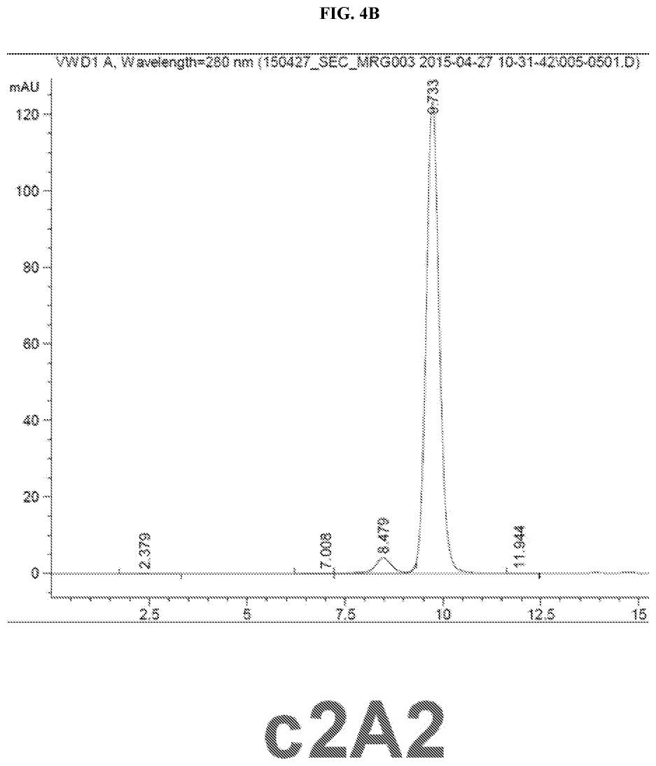

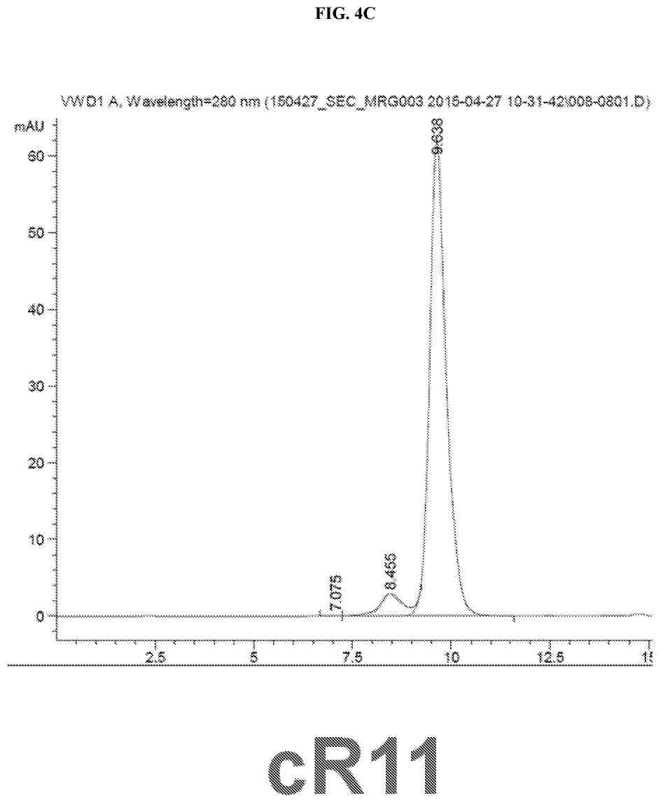

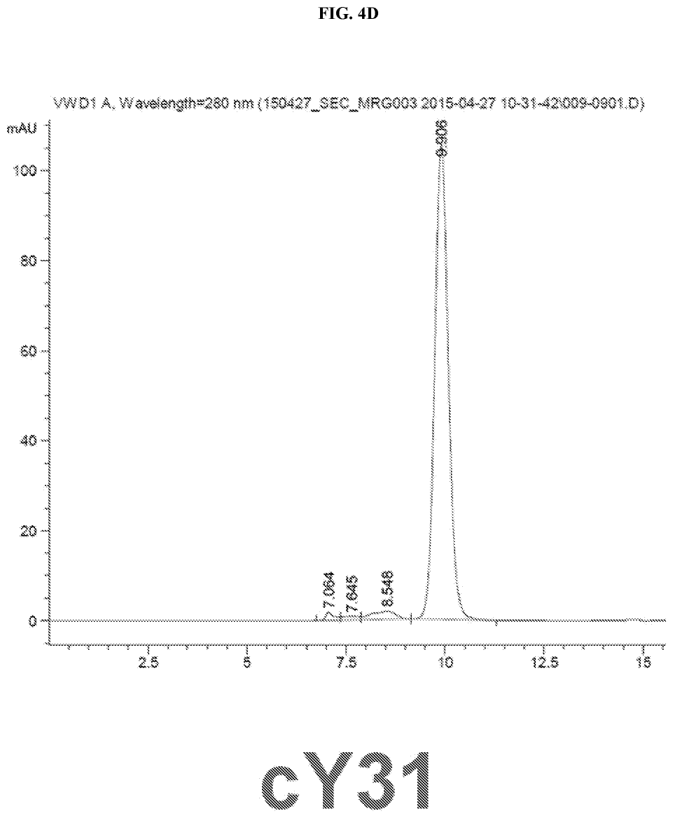

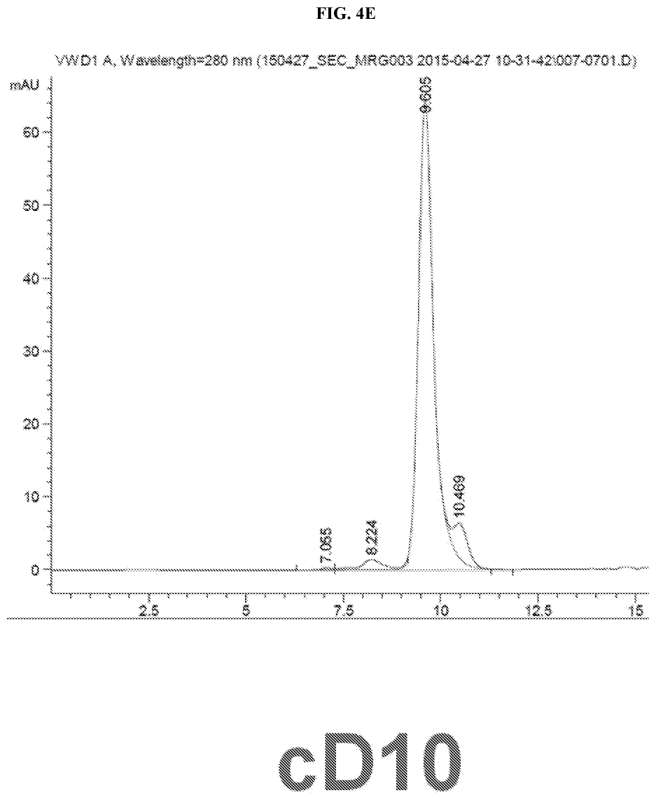

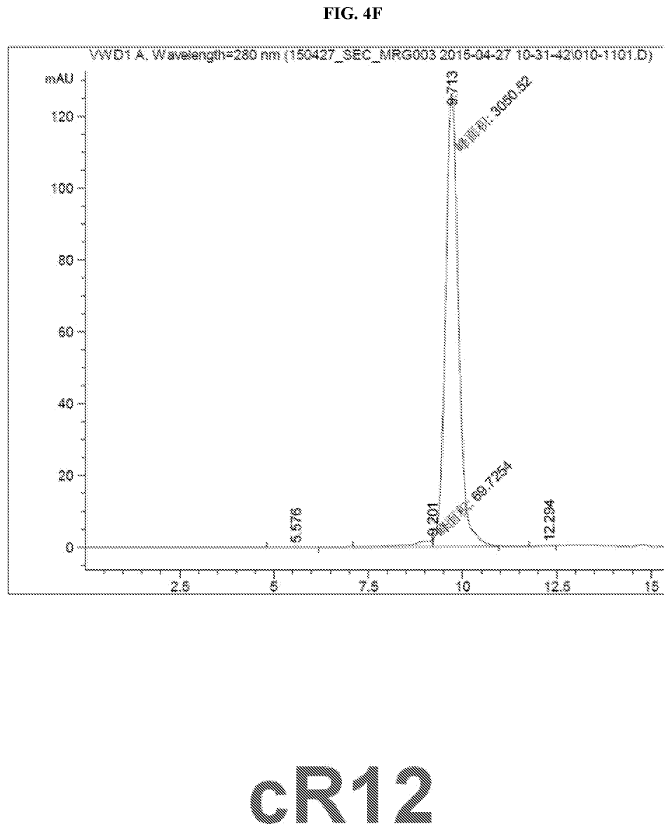

Seven antibodies, six anti-ROR1 antibodies and one control, were identified and production targets provided (Table 5). Variable heavy (VH) and variable light (VL) chains were cloned into plasmids which served as expression vectors to produce the 7 antibodies. The resulting plasmids, H-chain plasmid and L-chain plasmid respectively, were transfected and expressed in HEK293 cells with fectin. The quality and quantity of purified IgG was analyzed by SDS-PAGE (FIG. 3) and A₂₈₀ absorbance (FIGS. 4A-4F), respectively. These results demonstrate the actual production of IgG antibodies to ROR1, summarized in Table 6. Epitope recognition of anti-ROR1 antibodies were further characterized (Table 7).

TABLE 5
Summary of production targets for 7 antibodies.

| | | | Constant | Target |
|---|---|---|---|---|
| No. | Name | Description | Region | (mg) |
| 1 | m2A2 | Mouse 2A2 Antibody | mIgG1, mκ | 100 |
| 2 | h2A2 | Mouse/human chimeric 2A2 | hIgG1, hκ | 50 |
| 3 | h2A2m | Mouse/human chimeric 2A2 mutant | hIgG1, hκ | 50 |
| 4 | D10 | Murine D10 antibody (control) | mIgG1, mκ | 50 |
| 5 | R11 | Rabbit/human chimeric R11 | hIgG1, hκ | 50 |
| 6 | Y31 | Rabbit/human chimeric Y31 | hIgG1, hκ | 50 |
| 7 | R12 | Rabbit/human chimeric R12 | hIgG1, hλ | 50 |

TABLE 6
Summary of actual production for 7 antibodies. Concentrations were
calculated based on a universal molar extinction coefficient: 1.4
UV280 = 1 mg/ml IgG. All purified Abs were stored in PBS with
5% trehalose and 0.22 μM membrane filter sterilized.

| | | | Concentration | Volume | Amount |
|---|---|---|---|---|---|
| No. | Name | UV280 | (mg/ml) | (ml) | (mg) |
| 1 | m2A2 | 5.3 | 3.79 | 32 | 12.1 |
| 2 | h2A2 | 3.9 | 2.79 | 26 | 72.4 |
| 3 | h2A2m | 2.34 | 1.67 | 36.5 | 61.0 |
| 4 | D10 | 2.58 | 1.84 | 28 | 51.6 |
| 5 | R11 | 2.53 | 1.81 | 36 | 65.1 |
| 6 | Y31 | 3.56 | 2.54 | 24 | 61.0 |
| 7 | R12** | 4.03 | 2.88 | 3 | 8.6 |

**R12 expression issue isolated to light chain sequence.

TABLE 7
Characterization of anti-ROR1 antibodies.

| | | Light | | Binds | Internalize upon |
|---|---|---|---|---|---|
| Antibody | Form | Chain | K_{D} (pM) | mouse | ROR1 binding |
| 2A2 | Murine | Kappa | 100-400 | Yes | Yes; rapid |
| R11 | Rabbit/human | Kappa | 2700 | Yes | Yes; rapid |
| R12 | Rabbit/human | Lambda | 560 | No | Yes; slow |
| Y31 | Rabbit/human | Kappa | 8800 | Yes | Yes; rapid |

### Example 4: Conjugation of 4 Anti-ROR1 Antibody-Drug Conjugates (ADCs)

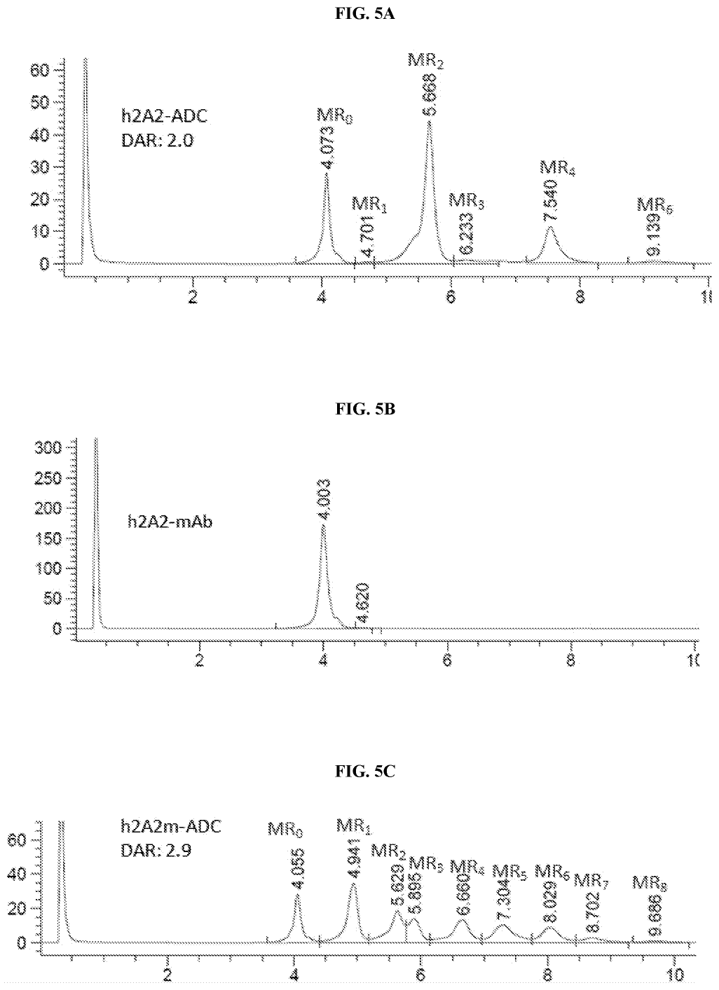

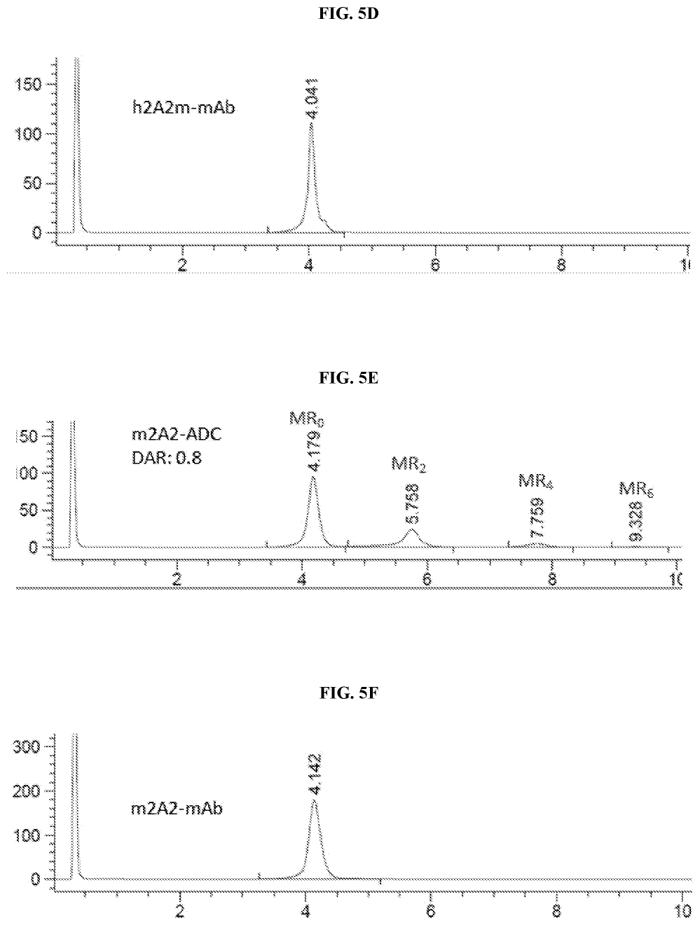

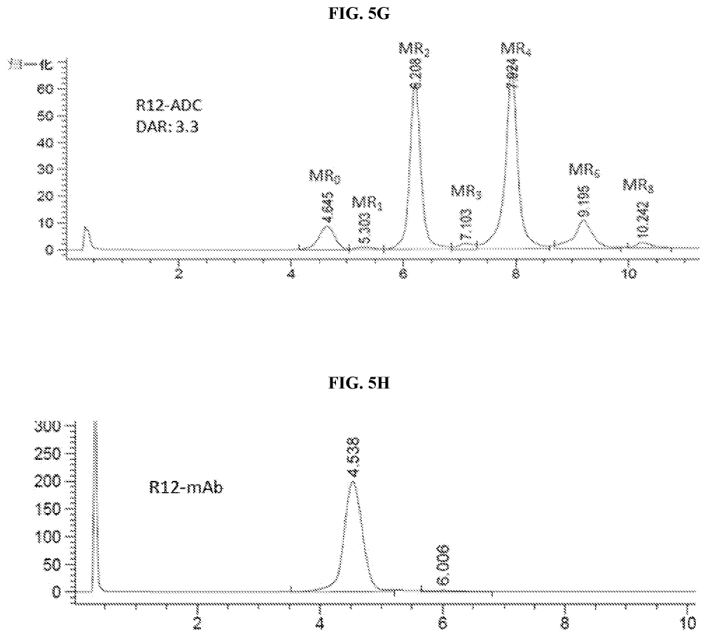

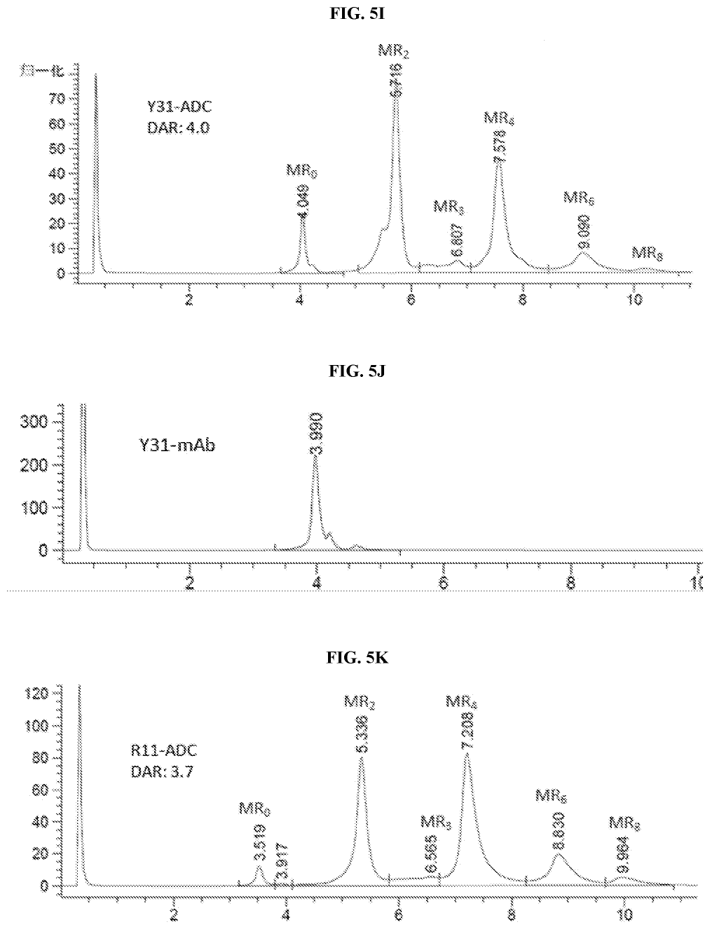

Four mAbs h2A2, m2A2, Y31, and R11 were chosen for conjugation with mc-vc-PAB-MMAE. The resultant ADCs had target DARs of 4.0 and low aggregate content (Table 8). HIC analysis was also carried out for each monoclonal antibody and their corresponding antibody-conjugate (FIGS. 5A-5L).

TABLE 8
DAR of 4 anti-ROR1 antibodies and their corresponding ADC.

| | mAb | | | | ADC | | | | |
|---|---|---|---|---|---|---|---|---|---|
| mAh | Size variants | | | Amount | Size variants | | | | Amount |
| ID | HMW | Monomer | LMW | (mg) | HMW | Monomer | LMW | DAR | (mg) |
| h2A2 | 0.8% | 98.2% | 1.0% | 5.5 | 2.2% | 97.8% | 0.1% | 4.2 | 9.0 |
| m2A2 | 1.0% | 98.9% | 0.2% | 55.0 | 1.3% | 98.6% | 0.2% | 3.9 | 6.0 |
| Y31 | 7.7% | 92.2% | 0.1% | 5.0 | 7.3% | 92.6% | 0.1% | 3.9 | 5.4 |
| R11 | 1.5% | 98.4% | 0.1% | 21.0 | 1.7% | 98.2% | 0.1% | 3.8 | 8.9 |

### Example 5: JeKo-1 Internalization Assay

The following JeKo-1 internalization assay was used.
1. Harvest Jeko-1 cells (suspension): count and spin down required cell number for experiment in 15 ml falcon tube. Re-suspended in PBS+3% FBS in bulk and aliquoted 100 μl (500,000 cells) per tube.
2. 2 μg each ADC per tube were added from bulk dilution, incubated 4° C. (on ice) for 1 hour.
3. 3×wash in 3 ml PBS+3% FBS. Centrifuge set to 1200 RPM for 3 min spin. Carefully decanted and discarded supernatant at each step. Pulse vortexed cell pellets. Left equal volumes in tubes after final wash.
4. Placed one set of tubes at 37° C. for each Ab tested, included secondary only control set for comparison.
T₀=4° C., T₁=37° C. 30 min, T₁=37+ C., 25 hours
5. After incubation times were complete, removed tubes from incubator and keep on ice until all samples were collected.
6. Added FITC labelled secondary, incubate on ice 45 min.
7. 3×wash in 3 ml PBS+3% FBS. Centrifuge set to 1500 RPM for 5 min spin. Carefully decanted and discarded supernatant at each step. Pulse vortexed cell pellets. Left equal volumes in tubes after final wash.
8. Collected data on instrument

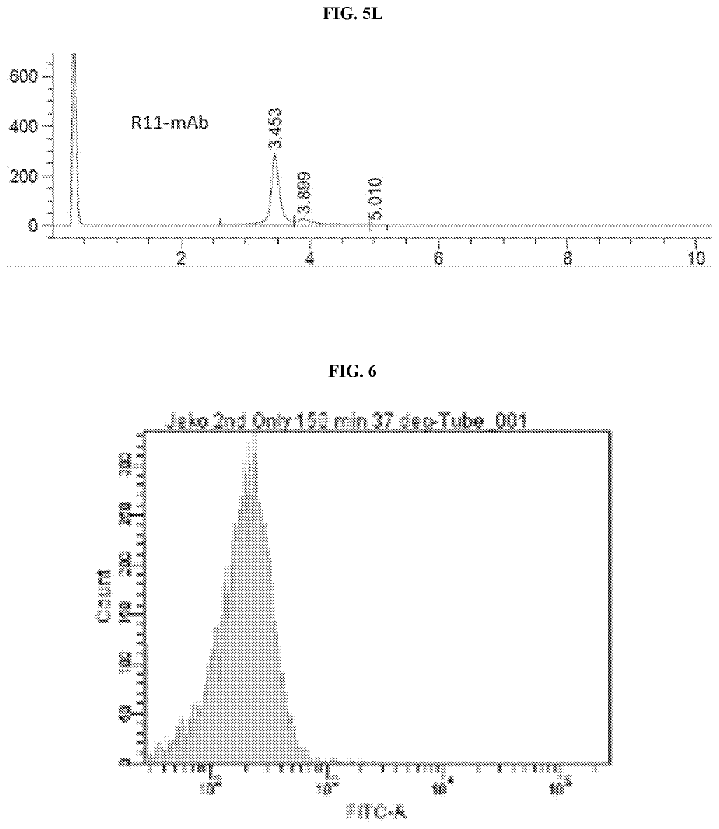

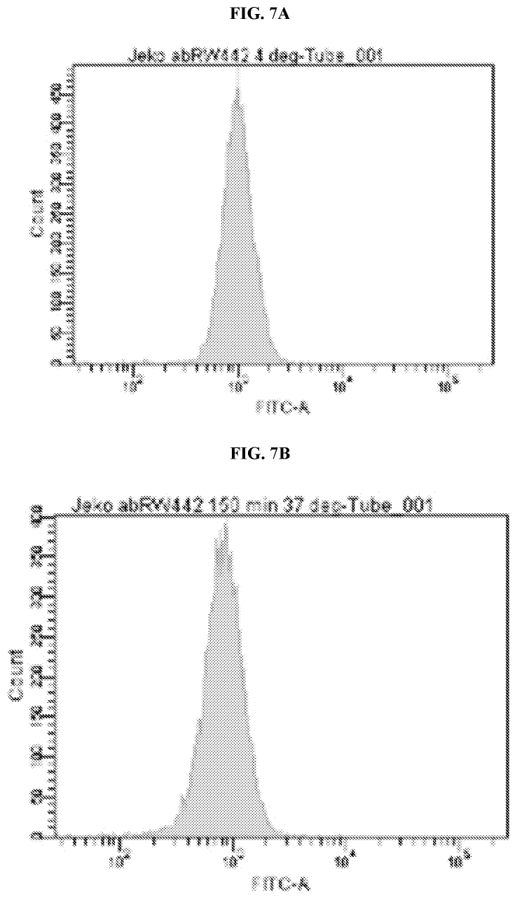

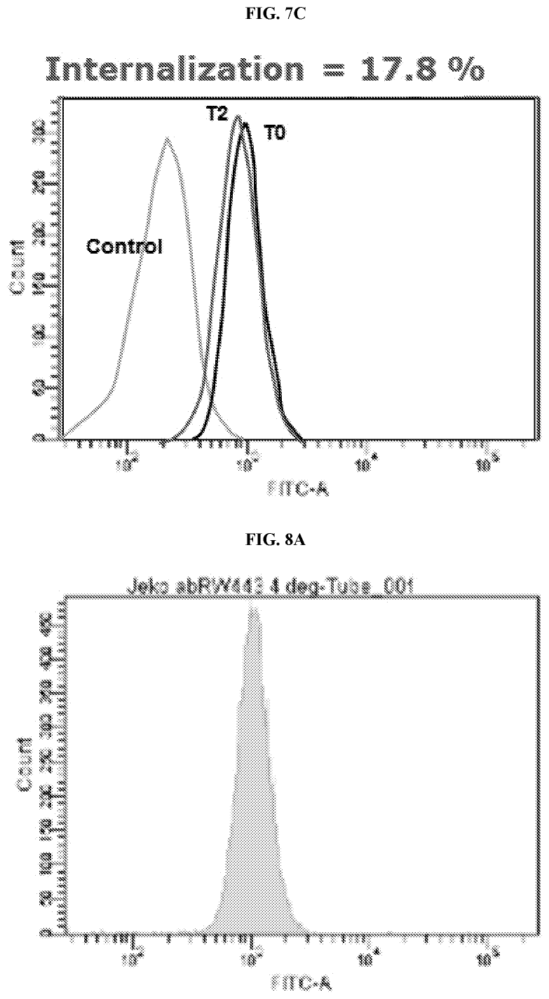

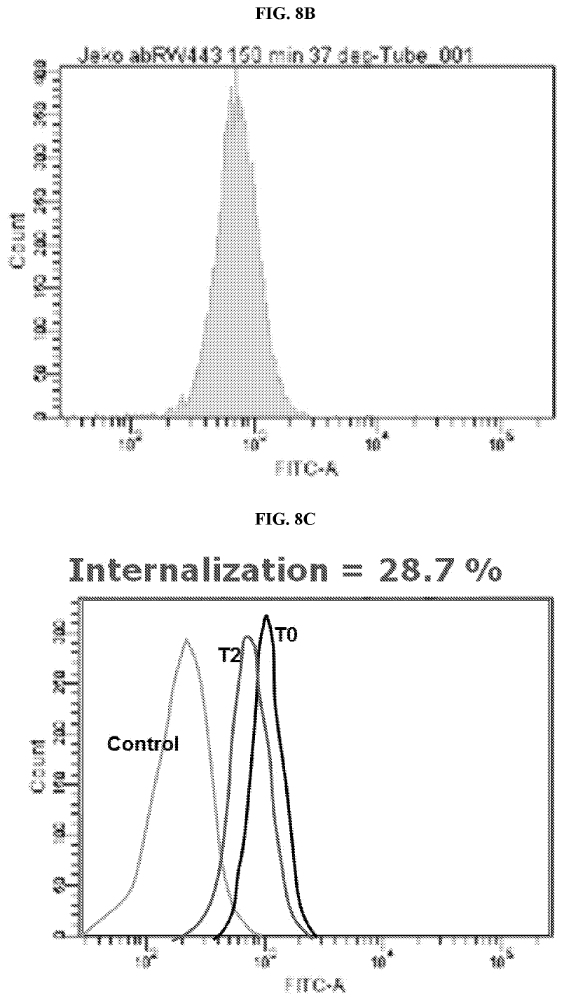

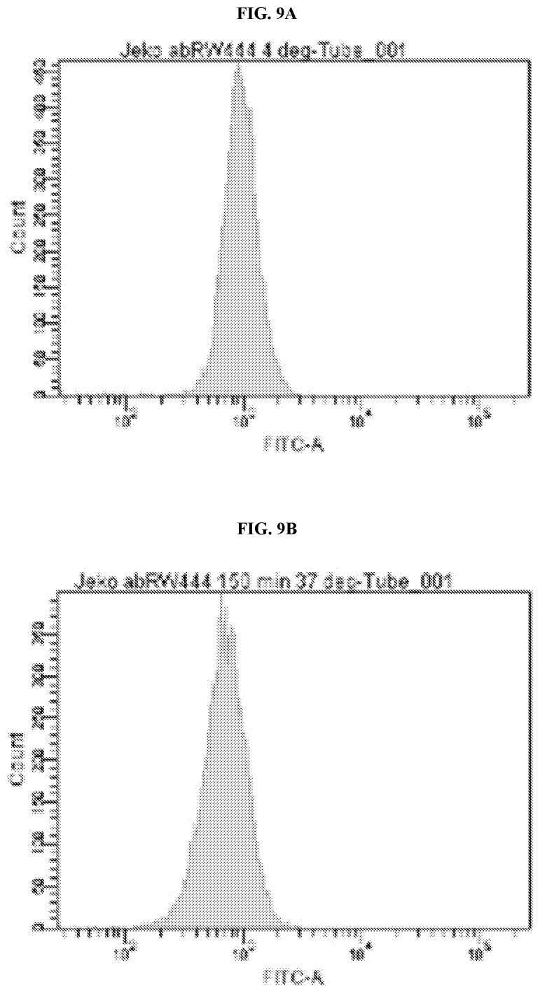

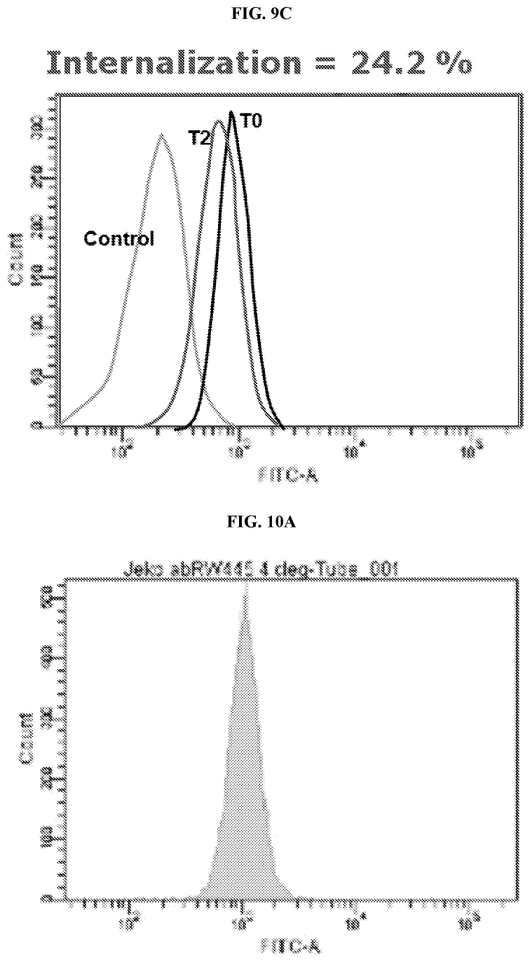

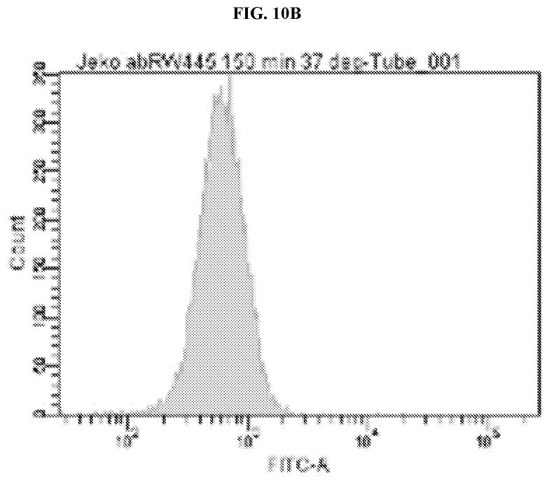

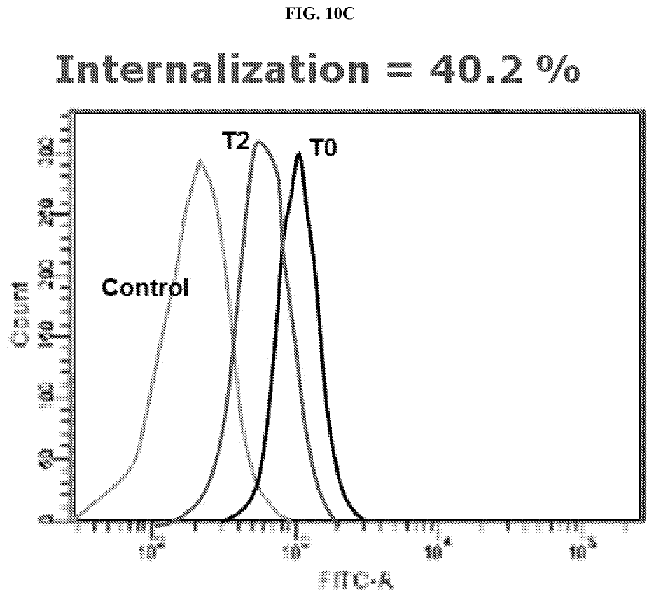

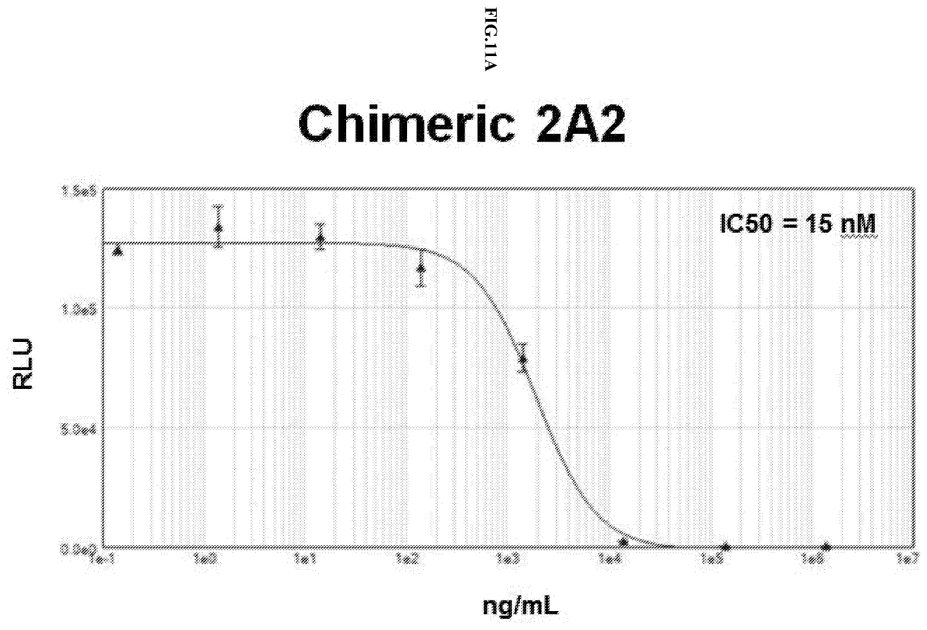

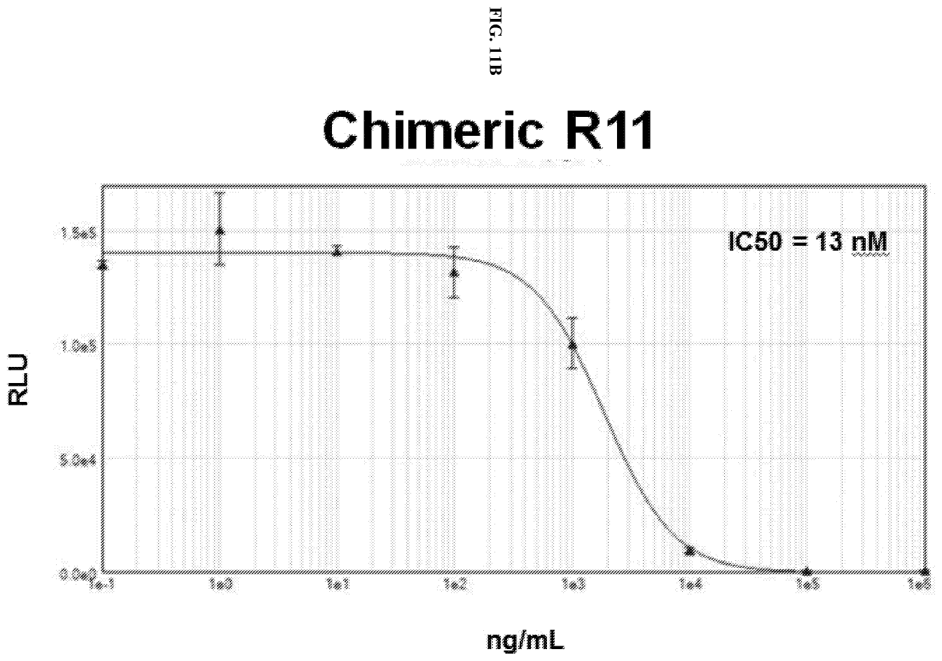

Naked antibodies were shown to internalize slowly while ADCs were shown to internalize relatively fast (Table 9) (FIGS. 6, 7A-C, 8A-C, 9A-C, 10A-C)

TABLE 9
Internalization results based on mean fluorescence intensity (MFI).

| Sample | Sample | | | Signal | |
|---|---|---|---|---|---|
| No. | Description | Condition | MFI | Δ | Internalization |
| 1 | Unstained | 4° C. (on ice) T0 | 122 | | |
| 2 | Secondary | 4° C. (on ice) T0 | 242 | | |
| 3 | only | 37° C., 30 min T1 | 216 | | |
| 4 | | 37° C., 2.5 hr T2 | 215 | | |
| 5 | 2A2- | 4° C. (on ice) T0 | 1073 | | |
| 6 | untagged | 37° C., 30 min T1 | 1071 | 2 | 2.0% |
| 7 | | 37° C., 2.5 hr T2 | 882 | 191 | 17.8% |
| 8 | 2A2-CH1 | 4° C. (on ice) T0 | 1103 | | |
| 9 | tag | 37° C., 30 min T1 | 1048 | 55 | 5.0% |
| 10 | | 37° C., 2.5 hr T2 | 786 | 317 | 28.7% |
| 11 | 2A2-C- | 4° C. (on ice) T0 | 990 | | |
| 12 | Terminal | 37° C., 30 min T1 | 959 | 31 | 3.1% |
| 13 | tag | 37° C., 2.5 hr T2 | 750 | 240 | 24.2% |
| 14 | 2A2-CH1/ | 4° C. (on ice) T0 | 1101 | | |
| 15 | C-Terminal | 37° C., 30 min T1 | 869 | 232 | 21.2% |
| 16 | double tag | 37° C., 2.5 hr T2 | 658 | 443 | 40.2% |

### Example 6: In Vitro Cytotoxicity

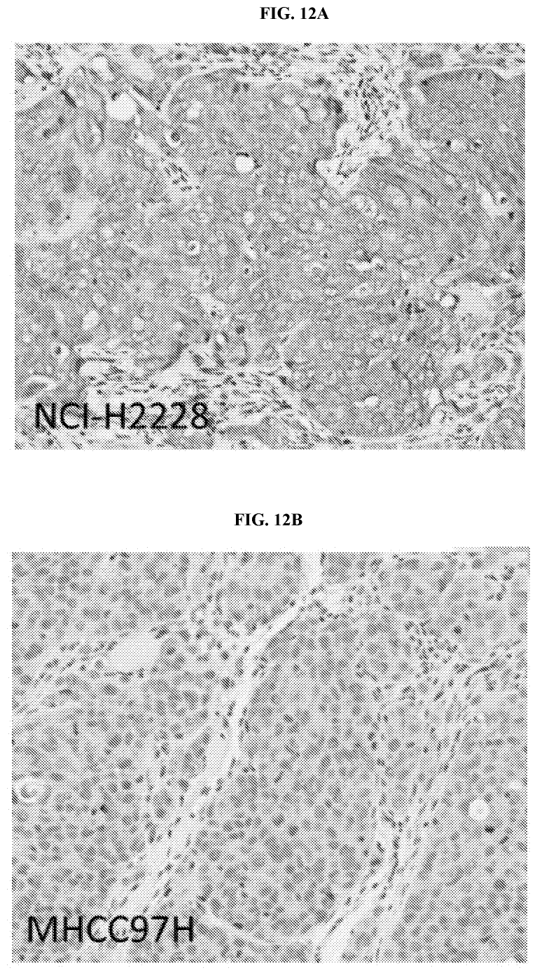

Two ADCs, 2A2-vcMMAE (4-load) and R11-vcMMAE (4-load), were examined for in vitro cytotoxicity towards the Jeko-1 cell line (FIGS. 11A and 11B).

### Example 7: ROR-1 Expression in Patient-Derived Tumor Xenografts

ROR-1 expression was examined in patient-derived tumor xenografts (in vivo PDX).

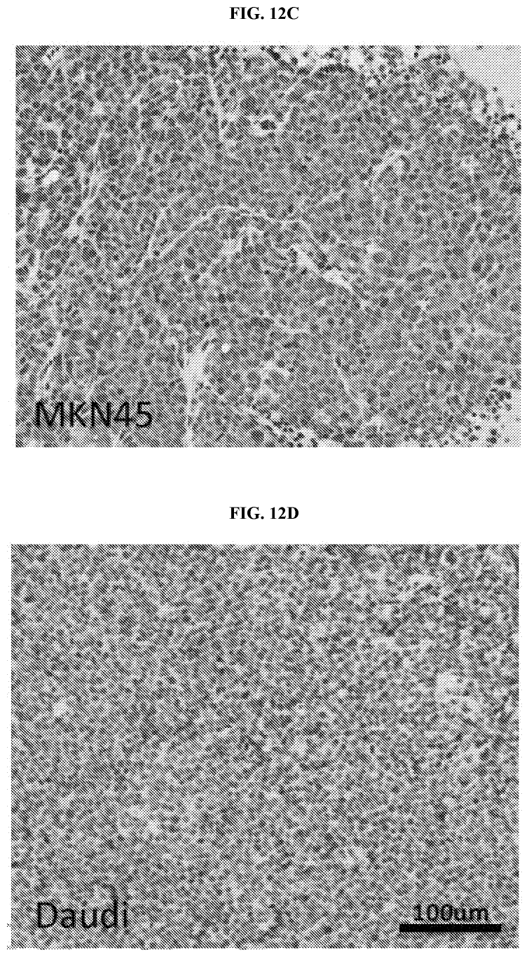

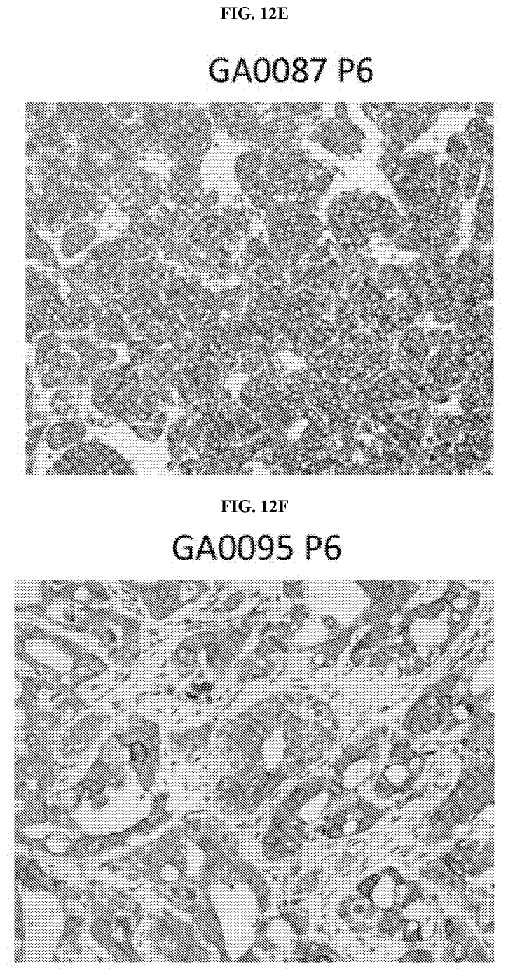

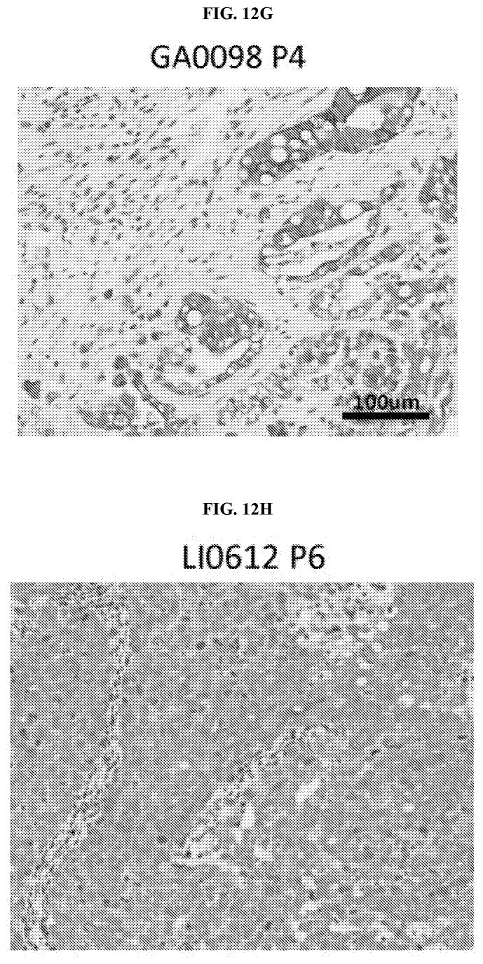

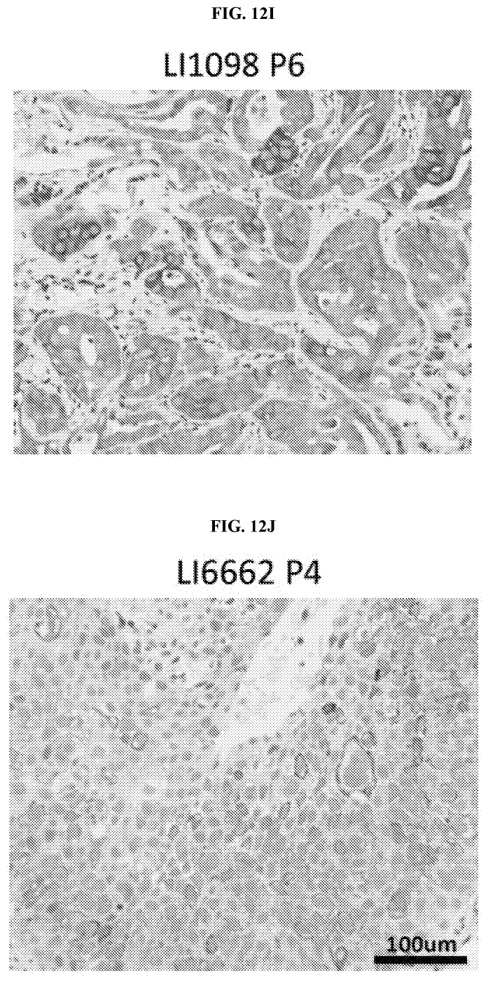

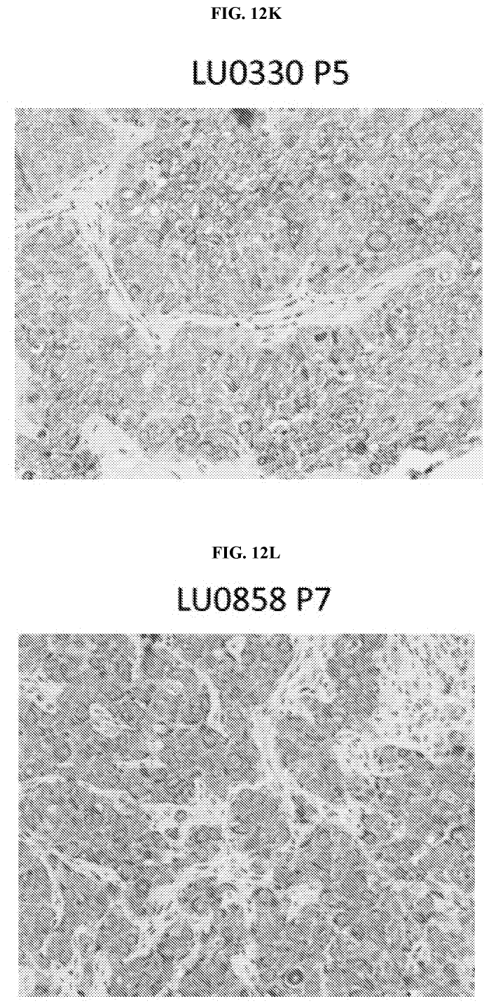

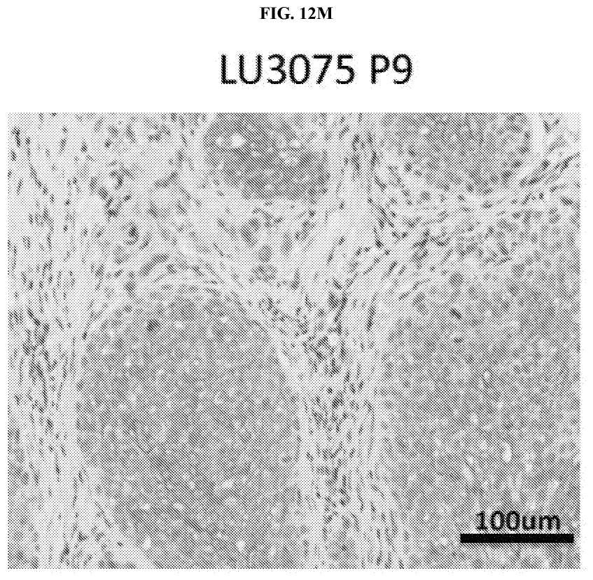

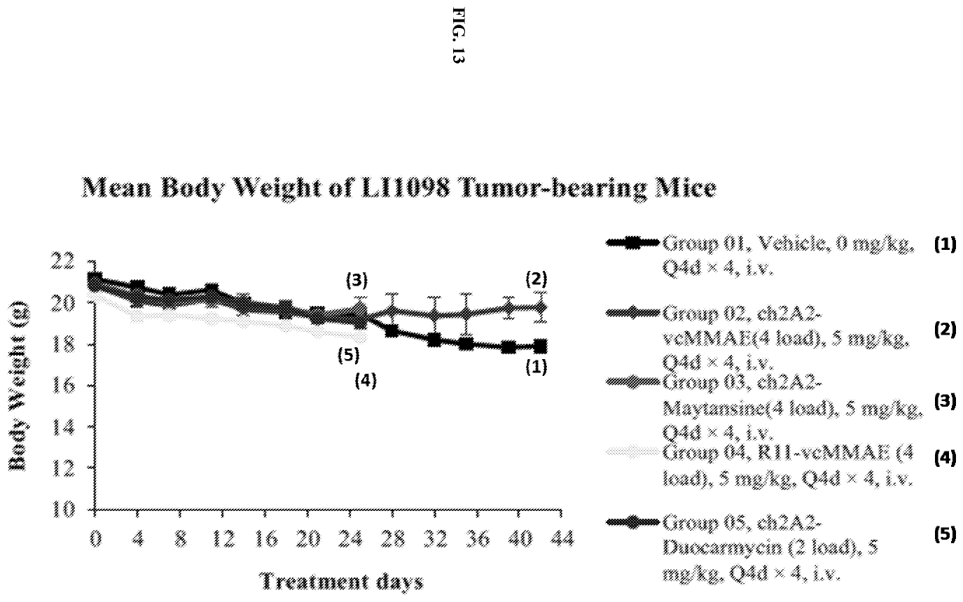

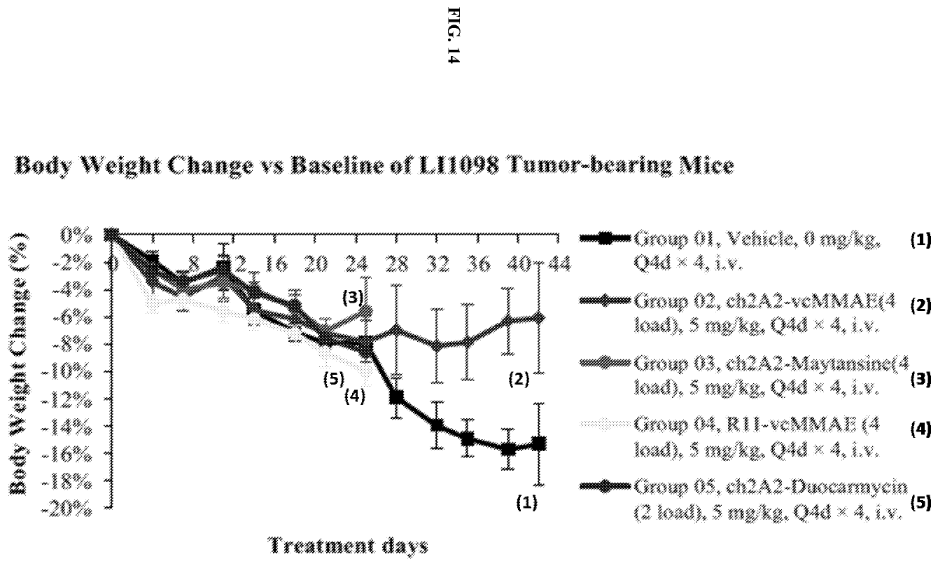

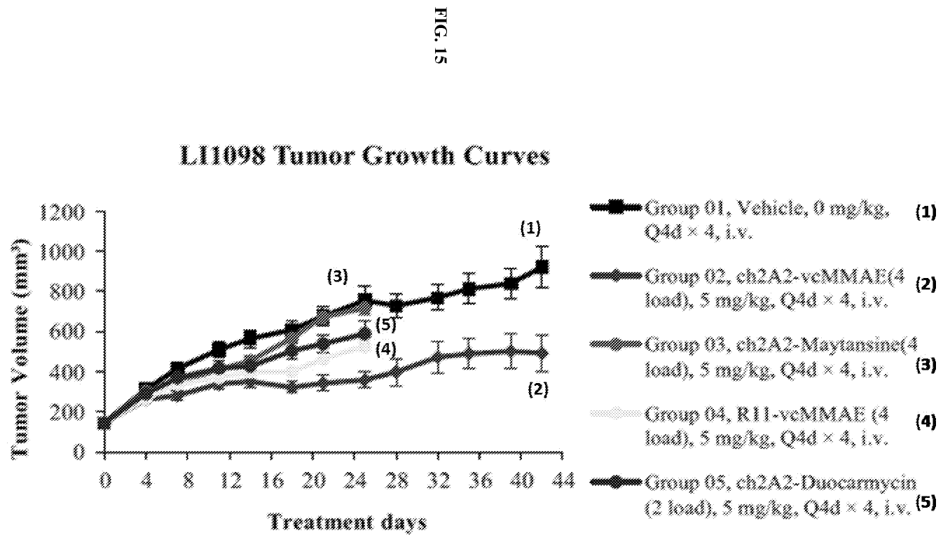

Section:
4 μm FFPE sections of NCI-H2228, MHCC97H, MKN45, Daui as control (FIGS. 12 A-D)
4 μm FFPE sections of PDX BR0438 and CDX Jeko-1 (lymphoma), NAMALWA (lymphoma), MDAMB231 (breast), NCI-H226 (lung), NCI-H2228 (lung), MCC97H (liver), HCCLM3 (liver)
TMA set 13 (FIGS. 12E-J)
TMA set 16 (FIGS. 12K-M)

Anti-ROR1 antibody, (1:500 dilution; at an initial concentration of 2 mg/ml) was incubated with the tissue at room temperature for 60 min. The tissue was then stained with Bond Polymer Refine Detection DS9800 Leica (with rabbit anti-mouse IgG).

### Example 8: PDX Study with Liver Cancer Line L11098

Anti-tumoral activity of ch2A2-vcMMAE (4 load), ch2A2-Maytansine (4 load), R11-vcMMAE (4 load) and ch2A2-Duocarmycin (2 load) were evaluated in LI1098 tumor-bearing mice.

### Materials and Methods

A total of 40 mice (Mus musculus; strain: BALB/c nude mice) were used. Female mice, 8-9 weeks at the start of treatment, were used.

HuPrime® liver cancer xenograft model LI1098 derived from a 51-year-old male Asian patient was selected. The pathology description for the LI1098 patient is: Hepatocellular carcinoma (HCC). LI1098 is a cachexia model and shows slow tumor growth rate.

### Experimental Methods and Procedures

Tumor fragments from stock mice inoculated with LI1098 primary human liver cancer tissues were harvested and used for inoculation into BALB/c nude mice. Each mouse was inoculated subcutaneously at the right flank with primary human liver cancer model LI1098 fragment (P5, 2-4 mm in diameter) for tumor development. The treatment was started when the average tumor size reached about 144 mm³. Mice were randomly allocated into 5 groups shown in Table 10, 8 mice per group. Start day was denoted as day 0. The test articles were administered to the tumor-bearing mice from day 0 through day 12 according to predetermined regimen shown in Table 10.

TABLE 10
Study design of LI1098 model.

| | | | | | Dosing | |
|---|---|---|---|---|---|---|
| Group | | | Dose | Dosing | volume | Dosing |
| ID | N | Compound | (mg/kg) | Route | (mL/kg) | Schedule |
| 1 | 8 | Vehicle | — | i.v. | 10 | Q4d x 4 |
| 2 | 8 | ch2A2-vcMMAE | 5 | i.v. | 10 | Q4d x 4 |
| | | (4 load) | | | | |
| 3 | 8 | ch2A2-Maytansine | 5 | i.v. | 10 | Q4d x 4 |
| | | (4 load) | | | | |
| 4 | 8 | R11-vcMMAE | 5 | i.v. | 10 | Q4d x 4 |
| | | (4 load) | | | | |
| 5 | 8 | ch2A2-Duocarmycin | 5 | i.v. | 10 | Q4d x 4 |
| | | (2 load) | | | | |

Detailed instructions on formulation and storage of the compounds administered to the mice are shown in Table 11.

TABLE 11
Detailed instructions on formulation and storage.

| | | | | Pre- |
|---|---|---|---|---|
| | Dose | | Concen- | pare |
| | (mg/ | | tration | fre- |
| Compound | kg) | Preparation | (mg/mL0 | quency |
| Vehicle | — | PBS + 5% trehalose | — | Directly |
| | | | | use |
| ch2A2- | 5 | Dilute 1.4 mL ch2A2-vcMMAE | 0.5 | Freshly |
| vcMMAE | | stock solution (0.7 mg/mL) with | | prepare |
| (4 load) | | 0.56 mL vehicle and mix well | | |
| ch2A2- | 5 | Dilute 0.45 mL cRW337 | 0.5 | Freshly |
| Maytansine | | stock solution (2.25 mg/mL) with | | prepare |
| (4 load) or | | 1.575 mL vehicle and mix well | | |
| cRW337 | | | | |
| R11- | 5 | Dilute 0.4 mL R11-vcMMAE | 0.5 | Freshly |
| vcMMAE | | stock solution (2.4 mg/mL) with | | prepare |
| (4 load) | | 1.52 mL vehicle and mix well | | |
| ch2A2- | 5 | Dilute 0.45 mL cRW339 stock | 0.5 | Freshly |
| Duocarmycin | | solution (2.33 mg/mL) with 1.647 | | prepare |
| (2 load) or | | mL vehicle and mix well | | |
| CRW339 | | | | |

Both of the T/C and tumor growth inhibition (TGI) were taken as endpoints to determine when the tumor growth is delayed or mice are be cured. Tumor size was measured twice weekly in two dimensions using a caliper, and the volume expressed in mm³ using the formula: V=0.5a×b², where a and b are the long and short diameters of the tumor, respectively. The tumor size is then used for calculations of both T/C and TGI values.

Six mice (Group I: 6379, 6386, 6418; Group 2: 6393, 6400, 6455) underwent extended observation and were terminated on day 42. The rest of the mice of this study were terminated on day 27.

Statistical analysis of difference in tumor volume among the groups was evaluated using one-way ANOVA followed by multiple comparison procedures with Tukey method when equal variances assumed or Games-Howell method when equal variance not assumed. All data were analyzed using SPSS 17.0. P<0.05 was considered to be statistically significant.

### Results

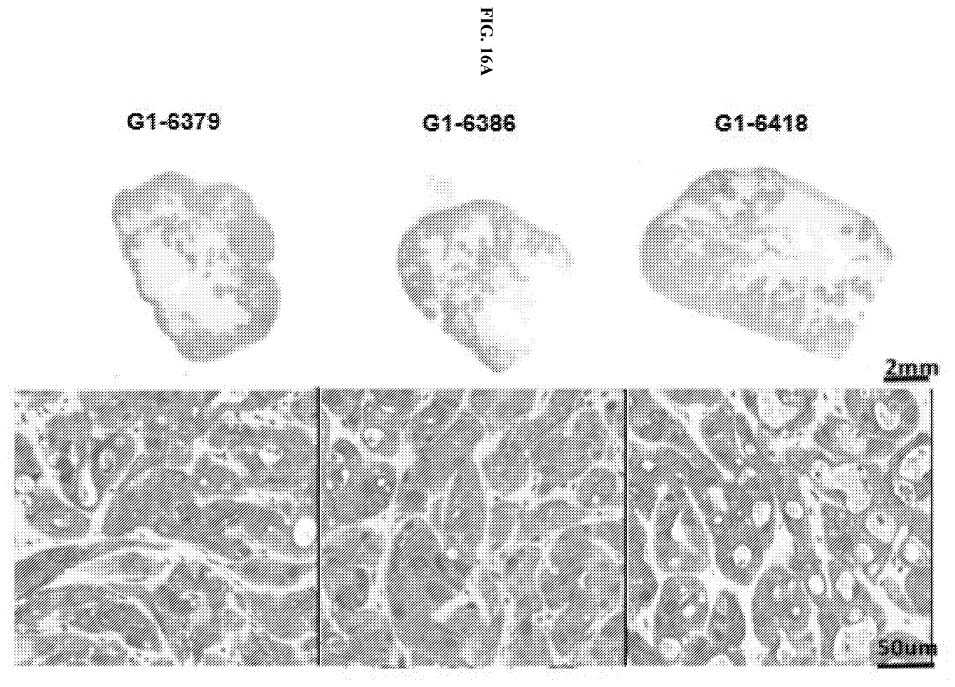

The results of body weights and body weight changes in the tumor bearing mice are shown in FIG. 13 and FIG. 14, respectively.

Tumor sizes of the different groups at different time points are shown in Table 12.

TABLE 12
Tumor sizes in the different treatment groups.

| | Tumor Volume (mm³) | | | | |
|---|---|---|---|---|---|
| | Group 01, | Group 02, | Group 03, | Group 04, | Group 05, |
| | Vehicle, | ch2A2-vcMMAE | ch2A2-Maytansine | R11-vcMMAE | ch2A2-Duocarmycin |
| | 0 mg/kg, | (4 load), 5 mg/kg, | (4 load), 5 mg/kg, | (4 load), 5 mg/kg, | (2 load), 5 mg/kg, |
| Days | Q4 d × 4, i.v. | Q4 d × 4, i.v. | Q4 d × 4, i.v. | Q4 d × 4, i.v. | Q4 d × 4, i.v. |
| 0 | 144.3 ± 7.1 | 144.3 ± 6.7 | 144.2 ± 7.6 | 144.2 ± 7.3 | 144.3 ± 7.3 |
| 4 | 310.5 ± 17.8 | 260.1 ± 20.6 | 302.8 ± 20.1 | 265.0 ± 12.1 | 290.1 ± 22.9 |
| 7 | 413.6 ± 30.5 | 281.0 ± 25.2** | 345.9 ± 24.8 | 337.2 ± 24.0 | 369.2 ± 25.4 |
| 11 | 509.1 ± 40.8 | 338.5 ± 27.5** | 409.4 ± 31.3 | 369.9 ± 21.3* | 417.5 ± 34.8 |
| 14 | 563.8 ± 44.5 | 350.0 ± 27.3*** | 453.2 ± 23.4 | 398.1 ± 21.7** | 427.0 ± 30.4* |
| 18 | 608.6 ± 47.7 | 321.4 ± 28.6** | 567.0 ± 28.1 | 399.5 ± 10.8* | 504.0 ± 41.5 |
| 21 | 675.6 ± 47.9 | 342.0 ± 37.8** | 672.1 ± 32.2 | 461.7 ± 12.2* | 538.8 ± 46.9 |
| 25 | 754.8 ± 68.6 | 358.5 ± 38.7** | 717.6 ± 31.2 | 526.0 ± 25.1 | 589.2 ± 66.0 |
| 28 | 729.1 ± 62.1 | 398.7 ± 67.5 | | — | — |
| 32 | 769.4 ± 64.3 | 471.7 ± 76.6 | | — | — |
| 35 | 812.5 ± 73.8 | 489.4 ± 76.5 | | — | — |
| 39 | 839.0 ± 75.9 | 500.9 ± 87.4 | | — | — |
| 42 | 921.2 ± 102.0 | 491.7 ± 92.5 | | — | — |

Note:
data expressed as Mean ± SEM;
*P < 0.05, **P < 0.01 and ***P < 0.001 compared with the vehicle control by one-way ANOVA followed by multiple comparison procedures with Tukey method when equal variance assumed (day 0 to day 14) or Games-Howell method when equal variance no assumed (day 18 to day 25).

Tumor growth inhibition is summarized in Table 13.

TABLE 13
Anti-tumor activity of the ADCs treatment in HuPrime ®.
liver cancer xenograft model LI1098

| | Tumor size | Tumor size | | | |
|---|---|---|---|---|---|
| | (mm³)^{a} on | (mm³)^{a} on | | | |
| | Day 0 of | Day 21 of | T/C | TGI | P |
| Treatment | treatment | treatment | (%)^{b} | (%)^{c} | valued^{d} |
| Group 01, Vehicle, | 144.3 ± | 675.6 ± | — | — | — |
| 0 mg/kg, | 7.1 | 47.9 | | | |
| Q4 d × 4, i.v. | | | | | |
| Group 02, ch2A2- | 144.3 ± | 342.0 ± | 50.6 | 62.8 | 0.001 |
| vcMMAE(4 load), | 6.7 | 37.8** | | | |
| 5 mg/kg, | | | | | |
| Q4 d × 4, i.v. | | | | | |
| Group 03, ch2A2- | 144.2 ± | 672.1 ± | 99.5 | 0.7 | 1.000 |
| Maytansine(4 load), | 7.6 | 32.2 | | | |
| 5 mg/kg, | | | | | |
| Q4 d × 4, i.v. | | | | | |
| Group 04, | 144.2 ± | 461.7 ± | 68.3 | 40.2 | 0.16 |
| R11-vcMMAE | 7.3 | 12.2* | | | |
| (4 load), 5 mg/kg, | | | | | |
| Q4 d × 4, i.v. | | | | | |
| Group 05, ch2A2- | 144.3 ± | 538.8 ± | 79.7 | 25.8 | 0.298 |
| Duocarmycin | 7.3 | 46.9 | | | |
| (2 load), 5 mg/kg, | | | | | |
| Q4 d × 4, i.v. | | | | | |

Note:
^{a}Mean ± SEM;
^{b}T/C % =T/C × 100%, where T and C are the mean tumor volume of the treated and control groups, respectively, on day 21;
^{c}TGI % = [1− (T₂₁ − T₀)/(C₂₁ − C₀)+ × 100%;
^{d}compared with the tumor volume of vehicle control by one-way ANOVA followed by multiple comparison procedures with Games-Howell method;
*P < 0.05 and **P < 0.01 compared with vehicle control.

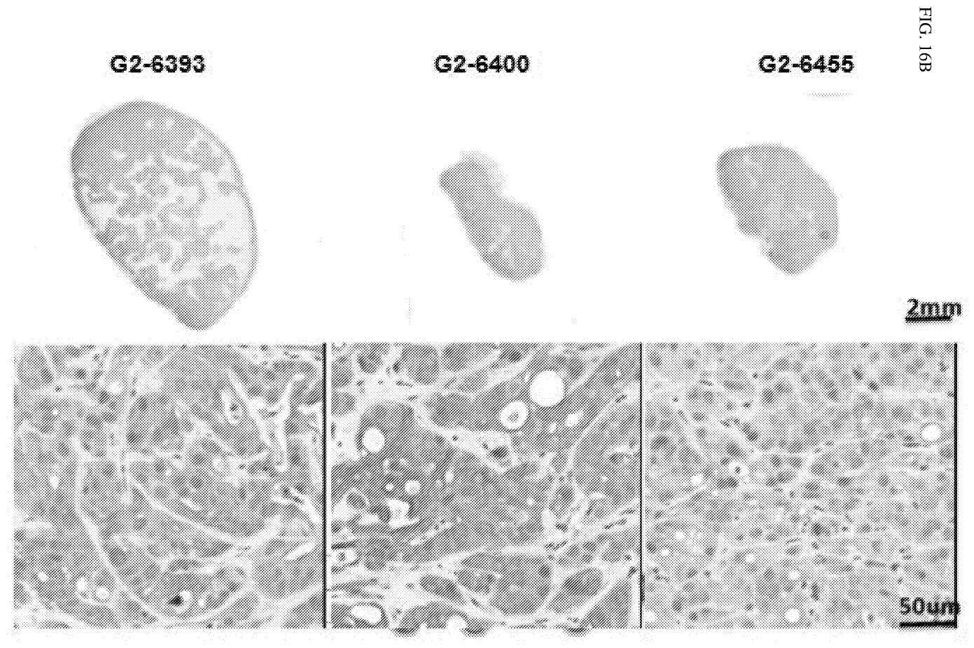

Tumor growth curves of different groups are shown in FIG. 15.

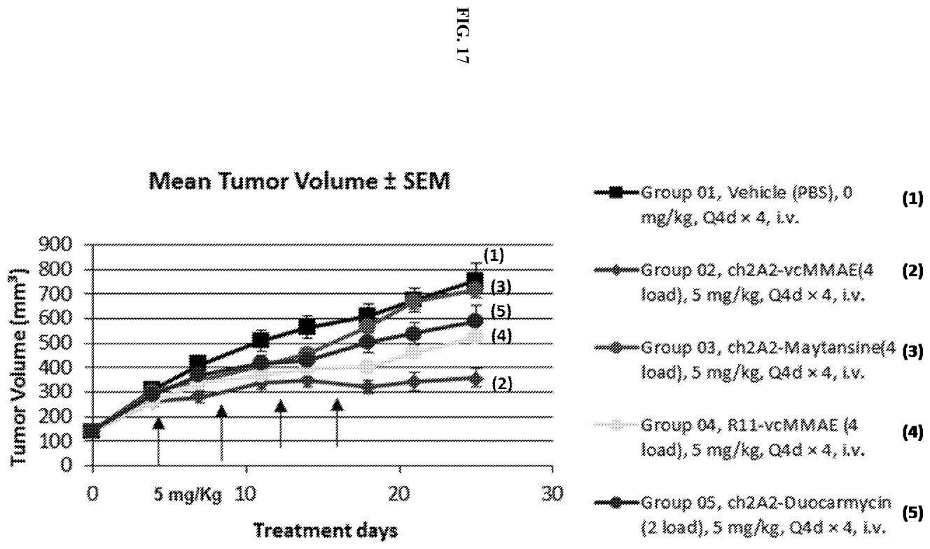

Tumors from six mice (Group 1: 6379, 6386, 6418; Group 2: 6393, 6400, 6455) were collected for ROR1 IHC analysis at study termination (FIGS. 16A-16B).

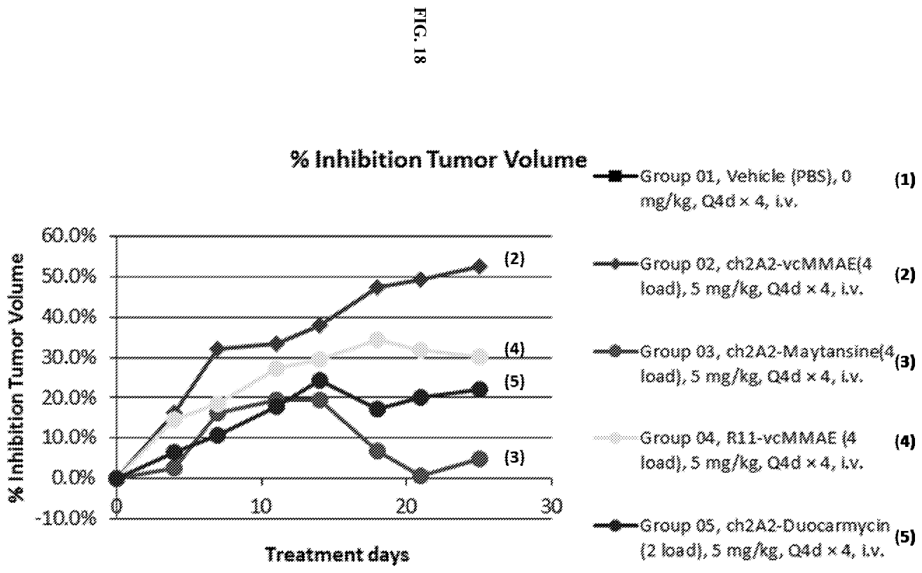

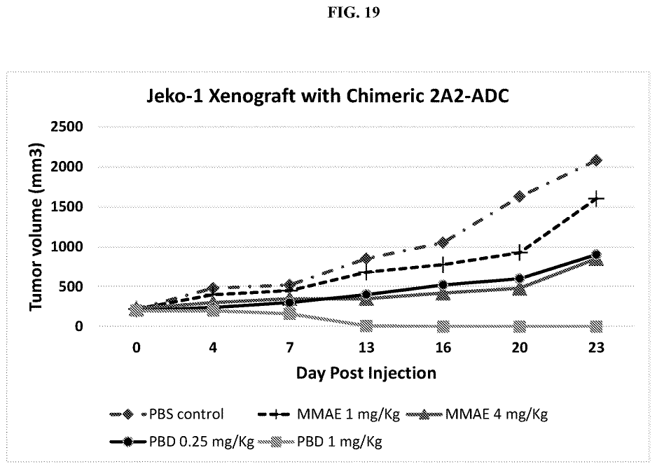

Mean tumor volume and percent inhibition of tumor volume is shown in FIGS. 17 and 18, respectively.

The test compounds ch2A2-vcMMAE (4 load), ch2A2-Maytansine (4 load), R11-vcMMAE (4 load), and ch2A2-Duocarmycin (2 load) were all tolerated by the LI1098 tumor-bearing mice in designated dose regimens. The mean maximum body weight loss (BWL) of the Group 01 (vehicle control), Group 02 (ch2A2-vcMMAE), Group 03 (ch2A2-Maytansine), Group 04 (R11-vcMMAE), and Group 05 (ch2A2-Duocarmycin) was −7.9% (on day 21), −7.8% (on day 25), −7.1% (on day 21), −9.9% (on day 25), and −8.6% (on day 25), respectively. The body weight loss of the study mice including vehicle control mice were mainly due to cachexia, which is a characteristic of the LI1098 model. Body weight measurements and body weight changes in different groups at different time points after treatment are shown in FIG. 13 and FIG. 14.

The mean tumor size of the vehicle treated mice reached 675.6 mm³ on day 21 post treatment initiation.

ch2A2-vcMMAE (4 load) exhibited considerable anti-tumor activity, treated at 5 mg/kg, Q4d resulted in a mean tumor size of 342.0 mm³ and a T/C ratio of 50.6% on day 21, which had significant different compared to the vehicle control (P<0.01) in terms of tumor volume.

ch2A2-Maytansine (4 load) exhibited no anti-tumor activity, treated at 5 mg/kg, Q4d resulted in a mean tumor size of 672.1 mm³ and a T/C ratio of 99.5% on day 21, which was similar to vehicle control group and no significant different was compared to the vehicle control (P>0.05) in terms of tumor volume.

R11-vcMMAE (4 load) exhibited medium anti-tumor activated, treated at 5 mg/kg, Q4d resulted in a mean tumor size of 461.7 mm³ and a T/C ratio of 68.3% on day 21, which had significant difference compared to the vehicle control (P<0.05) in terms of tumor volume.

ch2A2-Duocarmycin (2 load) exhibited minor anti-tumor activity, treated at 5 mg/kg, Q4d resulted in a mean tumor size of 538.8 mm³ and a T/C ratio of 79.7% on day 21, smaller than whereas no significant difference was observed compared to the vehicle control (P>0.05) in terms of tumor volume.

Overall, the results herein indicate that significant anti-tumor activity of the test compounds ch2A2-vcMMAE (4 load) and R11-vcMMAE (4 load) was observed against an L11098 HuPrime® liver cancer xenograft model in designed dose regimens, whereas no significant anti-tumor response of ch2A2-Maytansine (4 load) or ch2A2-Duocarmycin (2 load) was observed.

### Example 9: Patient-Derived Tumor Xenograft Study with Payload MMAE and PBD

Balb/c nude mice were implanted subcutaneously in the rear flank with the mantle cell lymphoma cell line Jeko-1 bearing the ROR1 antigen. On day 0 of the xenograft experiment the mice were divided into groups of 9 mice for each test group and were injected with a single dose of drug. The first group of mice received a vehicle control of PBS. The second group was injected with a single 1.0 mg/Kg dose of the chimeric 2A2 anti-ROR1 ADC conjugated with vcMMAE. The 3rd group was injected with a single 4.0 mg/Kg dose of the chimeric 2A2 anti-ROR1 ADC conjugated with vcMMAE. The 4th group was injected with a single 0.25 mg/Kg dose of the chimeric 2A2 anti-ROR1 ADC conjugated with PBD. The 5th group was injected with a single 1.0 mg/Kg dose of the chimeric 2A2 anti-ROR1 ADC conjugated with PBD. The tumor volumes were measured by digital calipers on days 0, 5, 7, 13, 16, 20, and 23 post dosing. All mice were euthanized following the conclusion of the experiment on day 23.

The 2A2-ADCs conjugated with the PBD cytotoxin at 1 mg/Kg showed strong activity with complete regression in all nine mice. At 0.25 mg/Kg tumors growth was severely restricted for two weeks post dose and then started to grow at a reduced rate relative to the control population. The 2A2-ADCs conjugated with vcMMAE cytotoxin at 4 mg/Kg showed strong cytostatic activity with no substantial growth for one week following dose administration and then significantly reduced growth for 3 weeks following dose administration in all nine mice. At 1.0 mg/Kg tumors growth was delayed relative to the control group. Both 2A2-PBD and 2A2-vcMMAE showed a dose response effect with 2A2-PBD being the more potent of the two cytotoxins.

FIG. 19 illustrates change in tumor volume over days post injection in Jeko-1 Xenograft mice treated with chimeric 2A2 conjugates.

While preferred embodiments have been shown and described herein, it will be obvious to those skilled in the art that such embodiments are provided by way of example only. Numerous variations, changes, and substitutions may now occur. It should be understood that various alternatives to the embodiments described herein can be employed in practicing the described methods. It is intended that the following claims define the scope of the embodiments and that methods and structures within the scope of these claims and their equivalents be covered thereby.

1. An anti-ROR1 antibody-payload conjugate comprising an anti-ROR1 antibody conjugated to a payload, wherein the anti-ROR1 antibody recognizes an epitope located within the immunoglobulin (Ig) domain, the Frizzled domain, or the Kringle domain of human ROR1, and wherein the payload comprises an auristatin derivative, maytansine, a maytansinoid, a taxane, a calicheamicin, cemadotin, a duocarmycin, a pyrrolobenzodiazepine (PBD), a tubulysin, or a combination thereof.

2. The anti-ROR1 antibody-payload conjugate of claim 1, wherein the auristatin derivative is monomethyl auristatin E (MMAE) or monomethyl auristatin F (MMAF).

3. The anti-ROR1 antibody-payload conjugate of claim 1, wherein the maytansinoid comprises DM1 (mertansine) or DM4.

4. The anti-ROR1 antibody-payload conjugate of claim 1, wherein the pyrrolobenzodiazepine is a pyrrolobenzodiazepine dimer.

5. The anti-ROR1 antibody-payload conjugate of claim 1, wherein the payload comprises monomethyl auristatin E (MMAE).

6. The anti-ROR1 antibody-payload conjugate of claim 1, wherein the payload comprises a pyrrolobenzodiazepine dimer. (Original) The anti-ROR1 antibody-payload conjugate of claim 1, wherein the anti-ROR1 antibody-payload conjugate further comprises a linker moiety that attaches the anti-ROR1 antibody to the payload.

8. The anti-ROR1 antibody-payload conjugate of claim 7, wherein the linker moiety comprises:
a) a homobifunctional linker or a heterobifunctional linker;
b) a cleavable linker;
c) a non-cleavable linker; or
d) a valine-citrulline moiety.

9. The anti-ROR1 antibody-payload conjugate of claim 7, wherein the linker moiety further comprises p-aminobenzoic acid.

10. The anti-ROR1 antibody-payload conjugate of claim 1, wherein the anti-ROR1 antibody further comprises a formylglycine residue generated by a formylglycine-generating enzyme.

11. The anti-ROR1 antibody-payload conjugate of claim 10, wherein the payload is conjugated to the anti-ROR1 antibody at the formylglycine site.

12. The anti-ROR1 antibody-payload conjugate of claim 1, wherein the anti-ROR1 antibody comprises a heavy chain variable region comprising three complementarity determining regions (CDRs) and a light chain variable region comprising three CDRs, wherein the heavy chain variable region comprises
i) a variable heavy (VH) CDR1 comprising an amino acid sequence of SEQ ID NO: 3;
ii) a variable heavy (VH) CDR2 comprising an amino acid sequence of SEQ ID NO: 4; and
iii) a variable heavy (VH) CDR3 comprising an amino acid sequence of SEQ ID NO: 5; and
wherein the light chain variable region comprises
iv) a variable light (VL) CDR 1 comprising an amino acid sequence of SEQ ID NO: 6;
v) a variable light (VL) CDR 2 comprising an amino acid sequence of SEQ ID NO: 7; and
vi) a variable light (VL) CDR 3 comprising an amino acid sequence of SEQ ID NO: 8.

13. The anti-ROR1 antibody-payload conjugate of claim 1, wherein the anti-ROR1 antibody comprises a heavy chain variable region comprising three complementarity determining regions (CDRs) and a light chain variable region comprising three CDRs, wherein the heavy chain variable region comprises
i) a variable heavy (VH) CDR1 comprising an amino acid sequence of SEQ ID NO: 20;
ii) a variable heavy (VH) CDR2 comprising an amino acid sequence of SEQ ID NO: 21; and
iii) a variable heavy (VH) CDR3 comprising an amino acid sequence of SEQ ID NO: 22; and
wherein the light chain variable region comprises
iv) a variable light (VL) CDR 1 comprising an amino acid sequence of SEQ ID NO: 23;
v) a variable light (VL) CDR 2 comprising an amino acid sequence of SEQ ID NO: 24; and
vi) a variable light (VL) CDR 3 comprising an amino acid sequence of SEQ ID NO: 25.

14. The anti-ROR1 antibody-payload conjugate of claim 1, wherein the anti-ROR1 antibody comprises a heavy chain variable region comprising three complementarity determining regions (CDRs) and a light chain variable region comprising three CDRs, wherein the heavy chain variable region comprises
i) a variable heavy (VH) CDR1 comprising an amino acid sequence of SEQ ID NO: 30;
ii) a variable heavy (VH) CDR2 comprising an amino acid sequence of SEQ ID NO: 31; and
iii) a variable heavy (VH) CDR3 comprising an amino acid sequence of SEQ ID NO: 32; and
wherein the light chain variable region comprises
iv) a variable light (VL) CDR 1 comprising an amino acid sequence of SEQ ID NO: 33;
v) a variable light (VL) CDR 2 comprising an amino acid sequence of SEQ ID NO: 34; and
vi) a variable light (VL) CDR 3 comprising an amino acid sequence of SEQ ID NO: 35.

15. The anti-ROR1 antibody-payload conjugate of claim 1, wherein the anti-ROR1 antibody comprises a heavy chain variable region comprising three complementarity determining regions (CDRs) and a light chain variable region comprising three CDRs, wherein the heavy chain variable region comprises
i) a variable heavy (VH) CDR1 comprising an amino acid sequence of SEQ ID NO: 38;
ii) a variable heavy (VH) CDR2 comprising an amino acid sequence of SEQ ID NO: 39; and
iii) a variable heavy (VH) CDR3 comprising an amino acid sequence of SEQ ID NO: 40; and
wherein the light chain variable region comprises
iv) a variable light (VL) CDR 1 comprising an amino acid sequence of SEQ ID NO: 41;
v) a variable light (VL) CDR 2 comprising an amino acid sequence of SEQ ID NO: 42; and
vi) a variable light (VL) CDR 3 comprising an amino acid sequence of SEQ ID NO: 43.

16. The anti-ROR1 antibody-payload conjugate of claim 1, wherein the anti-ROR1 antibody comprises:
a) a heavy chain variable region having at least 80%, 85%, 90%, 95%, or 99% sequence identity to SEQ ID NO: 1, 9 or 13 and a light chain variable region having at least 80%, 85%, 90%, 95%, or 99% sequence identity to SEQ ID NO: 2, 10 or 14;
b) a heavy chain variable region having at least 80%, 85%, 90%, 95%, or 99% sequence identity to SEQ ID NO: 17 or 18 and a light chain variable region having at least 80%, 85%, 90%, 95%, or 99% sequence identity to SEQ ID NO: 19;
c) a heavy chain variable region having at least 80%, 85%, 90%, 95%, or 99% sequence identity to SEQ ID NO: 28 and a light chain variable region having at least 80%, 85%, 90%, 95%, or 99% sequence identity to SEQ ID NO: 29; or
d) a heavy chain variable region having at least 80%, 85%, 90%, 95%, or 99% sequence identity to SEQ ID NO: 36 and a light chain variable region having at least 80%, 85%, 90%, 95%, or 99% sequence identity to SEQ ID NO: 37.

17. The anti-ROR1 antibody-payload conjugate of claim 1, wherein the anti-ROR1 antibody comprises:
a) a heavy chain variable region of SEQ ID NO: 1 and a light chain variable region of SEQ ID NO: 2;
b) a heavy chain variable region of SEQ ID NO: 9 and a light chain variable region of SEQ ID NO: 10;
c) a heavy chain variable region of SEQ ID NO: 13 and a light chain variable region of SEQ ID NO: 14;
d) a heavy chain variable region of SEQ ID NO: 17 and a light chain variable region of SEQ ID NO: 19;
e) a heavy chain variable region of SEQ ID NO: 18 and a light chain variable region of SEQ ID NO: 19;
f) a heavy chain variable region of SEQ ID NO: 28 and a light chain variable region of SEQ ID NO: 29; or
g) a heavy chain variable region of SEQ ID NO: 36 and a light chain variable region of SEQ ID NO: 37.

18. (canceled)

19. (canceled)

20. (canceled)

21. (canceled)

22. (canceled)

23. (canceled)

24. An anti-ROR1 antibody comprising a heavy chain variable region having at least 80%, 85%, 90%, 95%, or 99% sequence identity to SEQ ID NO: 18 and a light chain variable region having at least 80%, 85%, 90%, 95% or 99% sequence identity to SEQ ID NO: 19.

25. A pharmaceutical composition comprising an anti-ROR1 antibody of claim 24, and an excipient.

26. (canceled)

27. A method of treating a subject having cancer, comprising:
administering to the subject a therapeutically effective amount of a pharmaceutical composition comprising an anti-ROR1 antibody-payload conjugate wherein the subject has bladder cancer, breast cancer, colorectal cancer, liver cancer, lung cancer, pancreatic cancer, renal cell carcinoma, stomach cancer, adrenal cancer, skin cancer, prostate cancer, B-cell lymphoma, or acute lymphoblastic leukemia.

28.-81. (canceled)


SEQUENCE LISTING (48 sequences)

SEQ ID NO: 1 - protein, 118 aa, Artificial Sequence
  Description of Artificial Sequence Synthetic polypeptide
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 2 - protein, 107 aa, Artificial Sequence
  Description of Artificial Sequence Synthetic polypeptide
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 3 - protein, 5 aa, Artificial Sequence
  Description of Artificial Sequence Synthetic peptide
DYEMH

SEQ ID NO: 4 - protein, 17 aa, Artificial Sequence
  Description of Artificial Sequence Synthetic peptide
AIDPETGGTAYNQKFKG

SEQ ID NO: 5 - protein, 9 aa, Artificial Sequence
  Description of Artificial Sequence Synthetic peptide
YYDYDSFTY

SEQ ID NO: 6 - protein, 11 aa, Artificial Sequence
  Description of Artificial Sequence Synthetic peptide
KASQNVDAAVA

SEQ ID NO: 7 - protein, 7 aa, Artificial Sequence
  Description of Artificial Sequence Synthetic peptide
SASNRYT

SEQ ID NO: 8 - protein, 9 aa, Artificial Sequence
  Description of Artificial Sequence Synthetic peptide
QQYDIYPYT

SEQ ID NO: 9 - protein, 107 aa, Artificial Sequence
  Description of Artificial Sequence Synthetic polypeptide
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTY

SEQ ID NO: 10 - protein, 97 aa, Artificial Sequence
  Description of Artificial Sequence Synthetic polypeptide
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYT

SEQ ID NO: 11 - protein, 461 aa, Artificial Sequence
  Description of Artificial Sequence Synthetic polypeptide
MEWSRVFIFLLSVTAGVHSQVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 234 aa, Artificial Sequence
  Description of Artificial Sequence Synthetic polypeptide
MRCLAEFLGLLVLWIPGAIGDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - protein, 107 aa, Artificial Sequence; identical to SEQ ID NO: 9
  Description of Artificial Sequence Synthetic polypeptide

SEQ ID NO: 14 - protein, 97 aa, Artificial Sequence; identical to SEQ ID NO: 10
  Description of Artificial Sequence Synthetic polypeptide

SEQ ID NO: 15 - protein, 467 aa, Artificial Sequence
  Description of Artificial Sequence Synthetic polypeptide
MEWSRVFIFLLSVTAGVHSQVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 234 aa, Artificial Sequence
  Description of Artificial Sequence Synthetic polypeptide
MRCLAEFLGLLVLWIPGAIGDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 116 aa, Artificial Sequence
  Description of Artificial Sequence Synthetic polypeptide
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYCDFNIWGPGTLVTISS

SEQ ID NO: 18 - protein, 116 aa, Artificial Sequence
  Description of Artificial Sequence Synthetic polypeptide
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYSDFNIWGPGTLVTISS

SEQ ID NO: 19 - protein, 110 aa, Artificial Sequence
  Description of Artificial Sequence Synthetic polypeptide
ELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 20 - protein, 5 aa, Artificial Sequence
  Description of Artificial Sequence Synthetic peptide
DYPIS

SEQ ID NO: 21 - protein, 16 aa, Artificial Sequence
  Description of Artificial Sequence Synthetic peptide
FINSGGSTWYASWVKG

SEQ ID NO: 22 - protein, 11 aa, Artificial Sequence
  Description of Artificial Sequence Synthetic peptide
GYSTYYCDFNI

SEQ ID NO: 23 - protein, 11 aa, Artificial Sequence
  Description of Artificial Sequence Synthetic peptide
QASQSIDSNLA

SEQ ID NO: 24 - protein, 7 aa, Artificial Sequence
  Description of Artificial Sequence Synthetic peptide
RASNLAS

SEQ ID NO: 25 - protein, 12 aa, Artificial Sequence
  Description of Artificial Sequence Synthetic peptide
LGGVGNVSYRTS

SEQ ID NO: 26 - protein, 465 aa, Artificial Sequence
  Description of Artificial Sequence Synthetic polypeptide
MEFGLSWVFLVALLRGVQCQSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYSDFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 237 aa, Artificial Sequence
  Description of Artificial Sequence Synthetic polypeptide
METPAQLLFLLLLWLPDTTGELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 121 aa, Artificial Sequence
  Description of Artificial Sequence Synthetic polypeptide
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 29 - protein, 112 aa, Artificial Sequence
  Description of Artificial Sequence Synthetic polypeptide
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 30 - protein, 5 aa, Artificial Sequence
  Description of Artificial Sequence Synthetic peptide
AYYMS

SEQ ID NO: 31 - protein, 17 aa, Artificial Sequence
  Description of Artificial Sequence Synthetic peptide
TIYPSSGKTYYATWVNG

SEQ ID NO: 32 - protein, 12 aa, Artificial Sequence
  Description of Artificial Sequence Synthetic peptide
DSYADDGALFNI

SEQ ID NO: 33 - protein, 12 aa, Artificial Sequence
  Description of Artificial Sequence Synthetic peptide
TLSSAHKTDTID

SEQ ID NO: 34 - protein, 7 aa, Artificial Sequence
  Description of Artificial Sequence Synthetic peptide
GSYTKRP

SEQ ID NO: 35 - protein, 9 aa, Artificial Sequence
  Description of Artificial Sequence Synthetic peptide
GADYIGGYV

SEQ ID NO: 36 - protein, 117 aa, Artificial Sequence
  Description of Artificial Sequence Synthetic polypeptide
QSLEESGGRLVTPGTPLTLTCTVSGIDLNSHWMSWVRQAPGKGLEWIGIIAASGSTYYANWAKGRFTISKTSTTVDLRIASPTTEDTATYFCARDYGDYRLVTFNIWGPGTLVTVSS

SEQ ID NO: 37 - protein, 108 aa, Artificial Sequence
  Description of Artificial Sequence Synthetic polypeptide
ELVMTQTPSSVSAAVGGTVTINCQASQSIGSYLAWYQQKPGQPPKLLIYYASNLASGVPSRFSGSGSGTEYTLTISGVQREDAATYYCLGSLSNSDNVFGGGTELEIL

SEQ ID NO: 38 - protein, 5 aa, Artificial Sequence
  Description of Artificial Sequence Synthetic peptide
SHWMS

SEQ ID NO: 39 - protein, 16 aa, Artificial Sequence
  Description of Artificial Sequence Synthetic peptide
IIAASGSTYYANWAKG

SEQ ID NO: 40 - protein, 12 aa, Artificial Sequence
  Description of Artificial Sequence Synthetic peptide
DYGDYRLVTFNI

SEQ ID NO: 41 - protein, 11 aa, Artificial Sequence
  Description of Artificial Sequence Synthetic peptide
QASQSIGSYLA

SEQ ID NO: 42 - protein, 7 aa, Artificial Sequence
  Description of Artificial Sequence Synthetic peptide
YASNLAS

SEQ ID NO: 43 - protein, 10 aa, Artificial Sequence
  Description of Artificial Sequence Synthetic peptide
LGSLSNSDNV

SEQ ID NO: 44 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 45 - protein, 106 aa, Homo sapiens
PTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLF

SEQ ID NO: 46 - protein, 135 aa, Homo sapiens
EEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRI

SEQ ID NO: 47 - protein, 80 aa, Homo sapiens
KCYNSTGVDYRGTVSVTKSGRQCQPWNSQYPHTHTFTALRFPELNGGHSYCRNPGNQKEAPWCFTLDENFKSDLCDIPAC

SEQ ID NO: 48 - protein, 4 aa, Artificial Sequence
  Description of Artificial Sequence Synthetic peptide
LLQG